(19) United States
(12) Patent Application Publication
(10) Pub. No.: US 2008/0254470 A1
(43) Pub. Date: Oct. 16, 2008

(54) Methods and Nucleic Acids For the Analysis of Gene Expression Associated With the Prognosis of Cell Proliferative Disorders

(75) Inventor: Kurt Berlin, Stahnsdorf (DE)

Correspondence Address: DAVIS WRIGHT TREMAINE, LLP/Seattle, 1201 Third Avenue, Suite 2200, SEATTLE, WA, 98101-3045, US

(73) Assignee: Epigenomics AG, Berlin (DE)

(21) Appl. No.: 12/089,021

(22) PCT Filed: Oct. 4, 2006
(86) PCT No.: PCT/EP2006/009605

§ 371 Date: May 9, 2008

Related U.S. Application Data
(60) Provisional application No. 60/723,125, filed on Oct. 3, 2005.
(60) Provisional application No. 60/740,923, filed on Nov. 30, 2005.

(30) Foreign Application Priority Data
Jun. 15, 2006 (EP) 06090106.3

(51) Int. Cl. C12Q 1/68; G01N 33/574; C07H 21/00

(52) U.S. Cl. 435/6; 435/7.23; 536/23.1

(57) ABSTRACT

The present application provides methods and nucleic acids for providing a prognosis of cell proliferative disorders, most preferably cancer but not breast cancer.

### FIELD OF THE INVENTION

The present invention relates to human DNA sequences that exhibit heterogeneous expression patterns in cancer patients. Particular embodiments of the invention provide methods for determining the prognosis of said patients.

### PRIOR ART

The following invention relates to the use of the gene PITX2 as a prognostic marker in the treatment of cancer. The gene PITX2 (NM_{—}000325) encodes the paired-like homeodomain transcription factor 2 which is known to be expressed during development of anterior structures such as the eye, teeth, and anterior pituitary. In the state of the art it is known that hypermethylation and accordingly underexpression of this gene are associated with the development of cancer. Toyota et al., (2001. Blood. 97:2823-9.) found hypermethylation of the PITX2 gene in a large proportion of acute myeloid leukemias. However, this document does not disclose that the marker is also relevant in determining the prognosis of cancer patients. EP 04 029 486.0 is the closest single document relevant for the assessment of novelty. Said document discloses that PITX2 an indicator of breast cancer prognosis in EP 04 029 486.0. Said document does not disclose that said gene is a prognostic marker applicable across multiple types of cancer accordingly the invention is new.

Furthermore, on the basis of this document the person skilled in the art would not expect that said gene would also be a prognostic indicator in other cancerous disease. Due to the heterogeneity of cancers there is currently no single molecular prognostic indicator applicable across all classes of cancers. Accordingly the use of the gene PITX2 as a prognostic indicator across a plurality of cancer types is a surprising effect.

### SUMMARY OF THE INVENTION

The present invention provides novel and efficient methods and nucleic acids for providing a prognosis of cell proliferative disorders, most preferably cancer but not breast cancer. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

The invention solves this longstanding need in the art by providing the gene PITX2 (SEQ ID NO: 1) as a marker of cancer prognosis. In a particularly preferred embodiment of the invention, the methylation status of CpG positions of the gene PITX2 and/or regulatory regions thereof is indicative of the prognosis of cell proliferative disorders, most preferably cancer (but not breast cancer) or features thereof. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

To enable this analysis the invention provides a method for the analysis of biological samples for genomic methylation associated with the development of cell proliferative disorders, most preferably cancer but not breast cancer. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer. Said method is characterized in that at least one nucleic acid, or a fragment thereof of the gene PITX2 and/or regulatory regions thereof (SEQ ID NO: 1) is/are contacted with a reagent or series of reagents capable of distinguishing between methylated and non methylated CpG dinucleotides within the genomic sequence thereof.

It is particularly preferred that the method and nucleic acids according to the invention are utilized for at least one of: prognosis of; treatment of; monitoring of; and treatment and monitoring of cell proliferative disorders, most preferably cancer but not breast cancer. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

The present invention provides a method for ascertaining genetic and/or epigenetic parameters of genomic DNA. The method has utility for the improved prognostic classification of cell proliferative disorders, most preferably cancer but not breast cancer, more specifically by enabling the improved identification of and differentiation between aggressive and non-aggressive forms of said disorder. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

The invention presents several substantial improvements over the state of the art. Although some methylation assays for the detection of cancer are known, there is currently no molecular classification system for the prognostic classification of tumors.

The DNA source may be any suitable source. Preferably, the source of the DNA sample is selected from the group consisting of cells or cell lines, histological slides, biopsies, paraffin-embedded tissue, bodily fluids, ejaculate, urine, blood, sputum, stool, tissues for example but not limited to those from colon, prostate, lung or liver, and combinations thereof. Preferably, the source is biopsies, bodily fluids, ejaculate, urine, or blood.

Specifically, the present invention provides a method for providing a prognosis of cell proliferative disorders, most preferably cancer but not breast cancer, comprising: obtaining a biological sample comprising genomic nucleic acid(s); contacting the nucleic acid(s), or a fragment thereof, with one reagent or a plurality of reagents sufficient for distinguishing between methylated and non methylated CpG dinucleotide sequences within a target sequence of the subject nucleic acid, wherein the target sequence comprises, or hybridizes under stringent conditions to, a sequence comprising at least 16 contiguous nucleotides of SEQ ID NO: 1 said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences. Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NO: 2 to SEQ ID NO: 5, and contiguous regions thereof corresponding to the target sequence. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

Additional embodiments provide a method for providing a prognosis of cell proliferative disorders, most preferably cancer but not breast cancer, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting SEQ ID NO: 2 to SEQ ID NO: 5 and complements thereof, wherein the treated DNA or the fragment thereof is either amplified to produce an amplificate, or is not amplified; and determining, based on a presence or absence of, or on a property of said amplificate, the methylation state of at least one CpG dinucleotide sequence selected from the group consisting of SEQ ID NO:1, or an average, or a value reflecting an average methylation state of a plurality of CpG dinucleotide sequences thereof.

Preferably, at least one such hybridizing nucleic acid molecule or peptide nucleic acid molecule is bound to a solid phase. Preferably, determining comprises use of at least one method selected from the group consisting of: hybridizing at least one nucleic acid molecule comprising a contiguous sequence at least 9 nucleotides in length that is complementary to, or hybridizes under moderately stringent or stringent conditions to a sequence selected from the group consisting of SEQ ID NO:2 to SEQ ID NO:5, and complements thereof; hybridizing at least one nucleic acid molecule, bound to a solid phase, comprising a contiguous sequence at least 9 nucleotides in length that is complementary to, or hybridizes under moderately stringent or stringent conditions to a sequence selected from the group consisting of SEQ ID NO: 2 to SEQ ID NO: 5, and complements thereof; hybridizing at least one nucleic acid molecule comprising a contiguous sequence at least 9 nucleotides in length that is complementary to, or hybridizes under moderately stringent or stringent conditions to a sequence selected from the group consisting of SEQ ID NO: 2 to SEQ ID NO: 5, and complements thereof, and extending at least one such hybridized nucleic acid molecule by at least one nucleotide base; and sequencing of the amplificate.

Further embodiments provide a method for providing a prognosis of cell proliferative disorders, most preferably cancer but not breast cancer, comprising: obtaining a biological sample having subject genomic DNA; extracting the genomic DNA; contacting the genomic DNA, or a fragment thereof, comprising one or more sequences selected from the group consisting of SEQ ID NO:1 or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences selected from the group consisting of SEQ ID NO:1, or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within sequences from the group consisting of SEQ ID NO:1.

### BRIEF DESCRIPTION OF THE DRAWINGS

FIG. 1 shows the distribution of follow up times of patients as analyzed in Example 1. The white bars represent the distribution of all censored (no PSA relapse) patients. The grey bars show the distribution of the PSA-free survival time for all of the relapse patients. Frequency is shown on the Y-axis and time (months) is shown on the X-axis.

FIG. 2 shows Kaplan-Meier survival analysis of the PITX2 marker (A & B) and ROC curve analysis (C) of the marker PITX2 in differentiating between prostate cancer patients according to Example 1. Proportion of recurrence-free patients is shown on the Y-axis, time in years is shown on the x-axis.

FIG. 3 shows Kaplan-Meier survival analysis of PITX2 performance on sub-populations based on stage according to Example 1. Proportion of recurrence-free patients is shown on the Y-axis, time in years is shown on the x-axis. Figure A shows all T2 and T3 patients, wherein the dark grey plot shows clinical stage T3 patients, and light grey plot shows clinical stage T2 patients. Figure B shows all T3 patients, wherein the dark grey plot shows hypomethylated samples, and light grey plot shows hypomethylated samples. Figure C shows all T2 patients, wherein the dark grey plot shows hypomethylated samples, and light grey plot shows hypomethylated samples. Proportion of recurrence-free patients is shown on the Y-axis, time in years is shown on the x-axis.

FIG. 4 shows Kaplan-Meier survival analysis of PITX2 performance on sub-populations based on Gleason score according to Example 1. Figure A shows the performance of Gleason score as a prognostic marker. Gleason 5 and 6 patients are in light grey, Gleason 7 patients are in dark-grey, and Gleason 8, 9, and 10 patients are in black. Figure C shows the performance of PITX2 on Gleason 5 and 6 patients. Figure B shows the performance of PITX2 on Gleason 7 patients. Figure D shows the performance of PITX2 on Gleason 8, 9, and 10 patients. In figures B to D light grey shows hypomethylated samples, black indicates hypermethylated samples. Proportion of recurrence-free patients is shown on the Y-axis, time in years is shown on the x-axis.

FIG. 5 shows Kaplan-Meier survival analysis of PITX2 performance on sub-populations based on nomogram score according to Example 1. Figure A shows the performance of Nomogram score as a prognostic marker. High risk are in light grey, low risk patients are in black. Figure C shows the performance of PITX2 on high risk patients. Figure B shows the performance of PITX2 on low risk patients.

### DETAILED DESCRIPTION OF THE INVENTION

### Definitions

As used herein the term expression shall be taken to mean the transcription and translation of a gene. The level of expression of a gene may be determined by the analysis of any factors associated with or indicative of the level of transcription and translation of a gene including but not limited to methylation analysis, loss of heterozygosity (hereinafter also referred to as LOH), RNA expression levels and protein expression levels.

Furthermore the activity of the transcribed gene may be affected by genetic variations such as but not limited genetic mutations (including but not limited to SNPs, point mutations, deletions, insertions, repeat length, rearrangements and other polymorphisms).

The scope of the present invention is directed to cell proliferative disorders, more preferably cancers but not breast cancers. Accordingly the term “cancer but not breast cancer” and all equivalents thereof should be taken to mean all disorders of the group consisting of: Acute Lymphoblastic Leukemia; Acute Myeloid Leukemia; Adrenocortical Carcinoma; AIDS-Related Cancers; AIDS-Related Lymphoma; Anal Cancer; Astrocytoma (Cerebellar and Cerebral); Basal Cell Carcinoma; Bile Duct Cancer; Bladder Cancer; Bone Cancer, Osteosarcoma/Malignant Fibrous Histiocytoma; Brain Stem Glioma; Brain Tumor,—Brain Stem Glioma,—Cerebellar Astrocytoma,—Cerebral Astrocytoma/Malignant Glioma;—Ependymoma,—Medulloblastoma,—Supratentorial Primitive Neuroectodermal Tumors,—Visual Pathway and Hypothalamic Glioma; Bronchial Adenomas/Carcinoids; Burkitt's Lymphoma; Carcinoid Tumor; Carcinoid Tumor, Gastrointestinal; Central Nervous System Lymphoma, Primary; Cerebellar Astrocytoma; Cerebral Astrocytoma/Malignant Glioma; Cervical Cancer; Chronic Lymphocytic Leukemia; Chronic Myelogenous Leukemia; Chronic Myeloproliferative Disorders; Colon Cancer; Colorectal Cancer; Cutaneous T-Cell Lymphoma, Mycosis Fungoides and Sézary Syndrome; Endometrial Cancer; Ependymoma; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; GliomaBrain Stem; Glioma, Cerebral Astrocytoma; Glioma, Visual Pathway and Hypothalamic; Hairy Cell Leukemia; Head and Neck Cancer; Hepatocellular (Liver) Cancer (Primary); Hodgkin's Lymphoma; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Leukemia, Acute Lymphoblastic; Leukemia, Acute Myeloid; Leukemia, Chronic Lymphocytic; Lip and Oral Cavity Cancer; Liver Cancer (Primary); Lung Cancer, Non-Small Cell; Lung Cancer, Small Cell; Lymphoma, AIDS-Related; Lymphoma, Burkitt's; Lymphoma, Cutaneous T-Cell; Lymphoma, Hodgkin's; Lymphoma, Non-Hodgkin's; Lymphoma, Primary Central Nervous System; Macroglobulinemia, Waldenstrom's; Malignant Fibrous Histiocytoma of Bone/Osteosarcoma; Medulloblastoma; Melanoma; Melanoma, Intraocular (Eye); Merkel Cell Carcinoma; Mesothelioma Malignant; Mesothelioma; Metastatic Squamous Neck Cancer with Occult Primary; Multiple Endocrine Neoplasia Syndrome; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia Acute; Myeloma, Multiple; Myeloproliferative Disorders, Chronic; Nasal Cavity and Paranasal Sinus Cancer; Nasopharyngeal Cancer; Neuroblastoma; Non-Hodgkin's Lymphoma; Non-Small Cell Lung Cancer; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Retinoblastoma; Rhabdomyosarcoma; Salivary Gland Cancer; Sarcoma, Ewing's Family of Tumors; Sarcoma, Kaposi's; Sarcoma, Soft Tissue; Sarcoma, Uterine; Sezary Syndrome; Skin Cancer (non-Melanoma); Skin Cancer; Skin Cancer (Melanoma); Skin Carcinoma, Merkel Cell; Small Cell Lung Cancer; Small Intestine Cancer; Soft Tissue Sarcoma; Squamous Cell Carcinoma; Squamous Neck Cancer with Occult Primary; Stomach (Gastric) Cancer; Supratentorial Primitive Neuroectodermal Tumors; T-Cell Lymphoma, Cutaneous Testicular Cancer; Thymoma; Thymoma and Thymic Carcinoma; Thyroid Cancer; Transitional Cell Cancer of the Renal Pelvis and Ureter; Trophoblastic Tumor, Gestational; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma; Vulvar Cancer; Waldenstmm's Macroglobulinemia; and Wilms' Tumor.

As used herein the term “prognosis” shall be taken to mean a prediction of the progression of the disease (for example but not limited to regression, stasis and metastasis), in particular aggressiveness and metastatic potential of a tumor.

As used herein the term “prognostic marker” shall be taken to mean an indicator of a prediction of the progression of the disease, in particular aggressiveness and metastatic potential of a tumor.

As used herein the term “prognostic classification” or “prognosis” shall be taken to mean the classification of a cell proliferative disorder, preferably cancer but not breast cancer according to a prediction of the progression of the disease, in particular aggressiveness and metastatic potential of a tumor.

It is preferably used to define patients with high, low and intermediate risks of death or recurrence after treatment that result from the inherent heterogeneity of the disease process. As used herein the term “aggressive” as used with respect to a tumor shall be taken to mean a cell proliferative disorder that has the biological capability to rapidly spread outside of its primary location or organ. Indicators of tumor aggressiveness standard in the art include but are not limited to tumor stage, tumor grade, Gleason grade, nodal status and survival. As used herein the term “survival” shall not be limited to mean survival until mortality (wherein said mortality may be either irrespective of cause or cell proliferative disorder related) but may be used in combination with other terms to define clinical terms, for example but not limited to “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and a defined end point (e.g. death, recurrence or metastasis).

The term “Observed/Expected Ratio” (“O/E Ratio”) refers to the frequency of CpG dinucleotides within a particular DNA sequence, and corresponds to the [number of CpG sites/(number of C bases×number of G bases)].

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated” and “hemi-methylated.”

The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CCMGG-3′ (top strand): 3′-GGCC-5′ (bottom strand)).

The term ‘AUC’ as used herein is an abbreviation for the area under a curve. In particular it refers to the area under a Receiver Operating Characteristic (ROC) curve. The ROC curve is a plot of the true positive rate against the false positive rate for the different possible cutpoints of a diagnostic test. It shows the tradeoff between sensitivity and specificity depending on the selected cutpoint (any increase in sensitivity will be accompanied by a decrease in specificity). The area under an ROC curve (AUC) is a measure for the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.
The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The terms “array SEQ ID NO,” “composite array SEQ ID NO,” or “composite array sequence” refer to a sequence, hypothetical or otherwise, consisting of a head-to-tail (5′ to 3′) linear composite of all individual contiguous sequences of a subject array (e.g., a head-to-tail composite of SEQ ID NO:1-71, in that order).

The terms “array SEQ ID NO node,” “composite array SEQ ID NO node,” or “composite array sequence node” refer to a junction between any two individual contiguous sequences of the “array SEQ ID NO,” the “composite array SEQ ID NO,” or the “composite array sequence.”

In reference to composite array sequences, the phrase “contiguous nucleotides” refers to a contiguous sequence region of any individual contiguous sequence of the composite array, but does not include a region of the composite array sequence that includes a “node,” as defined herein above.

### Overview:

The present invention provides for a molecular genetic marker that has utility for providing a prognosis of cell proliferative disorders, most preferably cancer but not breast cancer. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer. In particular embodiments said marker may be used for classifying the cancer according to aggressiveness and/or invasiveness. It is particularly preferred that the method and nucleic acids according to the invention are utilized for at least one of: prognosis of; treatment of; monitoring of; and treatment and monitoring of cell proliferative disorders, most preferably cancer but not breast cancer.

The term ‘prognosis’ is taken to mean a prediction of outcome of disease progression (wherein the term progression shall be taken to also include recurrence after treatment). Prognosis may be expressed in terms of overall patient survival, disease- or relapse-free survival, increased tumor-related complications and rate of progression of tumor or metastases, wherein a decrease in any of said factors (with the exception of increased tumor-related complications rate of progression) as relative to a pre-determined level, is a ‘negative’ outcome and increase thereof is a ‘positive’ outcome. A decrease in tumor-related complications and/or rate of progression of tumor or metastases as relative to a pre-determined level, is considered a ‘positive’ outcome and increase thereof is a ‘negative’ outcome.

Hereinafter prognosis may also be referred to in terms of ‘aggressiveness’ wherein an aggressive cancer is determined to have a high risk of negative outcome and wherein a non-aggressive cancer has a low risk of negative outcome.

In one aspect the prognostic marker according to the present invention is used to provide an estimate of the risk of negative outcome. For example, characterization of a cancer in terms of predicted outcome enables the physician to determine the risk of recurrence and/or death. This aids in treatment selection as the absolute reduction of risk of recurrence and death after treatments such as adjuvant hormonal, chemo-, and radiation therapy can be determined based on the predicted negative outcome. The absolute reduction in risk attributable to treatment may then be compared to the drawbacks of said treatment (e.g. side effects, cost) in order to determine the suitability of said treatment for the patient.

Conversely, wherein a cancer is characterized as non-aggressive (i.e. positive outcome with low risk of death and/or recurrence) the patient will derive low absolute benefit from adjuvant or other treatment and may be appropriately treated by watchful waiting (commonly prescribed in prostate cancer). Therein lies a great advantage of the present invention. By providing a means for determining which patients will not significantly benefit from treatment the present prevents the over-prescription of therapies.

According to the predicted outcome (i.e. prognosis) of the disease an appropriate treatment or treatments may be selected. Wherein a cancer is characterized as aggressive it is particularly preferred that adjuvant treatment such as, but not limited to, hormonal, chemo- or radiation therapy is provided in addition to or instead of further treatments.

The herein described marker has further utility in predicting outcome of a patient after surgical treatment. This will hereinafter also be referred to as a ‘predictive’ marker. Over expression of the gene PITX2 is associated with negative outcome of cancer patients. Patients with predicted positive outcome (i.e. hypomethylation or over-expression) after said treatment will accordingly have a decreased absolute reduction of risk of recurrence and death after treatment with post surgical adjuvant therapies. Patients with predicted negative outcome (i.e. hypermethylation or under-expression) after said treatment will accordingly have a relatively larger absolute reduction of risk of recurrence and death after post surgical adjuvant treatment. Accordingly patients with a negative outcome after said treatment will be considered more suitable candidates for adjuvant treatment than patients with a positive outcome. Patients with a positive outcome may accordingly be prevented from over-prescription of adjuvant treatment.

Bisulfite modification of DNA is an art-recognized tool used to assess CpG methylation status. 5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is completely lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil, which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analyzed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyze individual cells for methylation status, illustrating the utility and sensitivity of the method. An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek & Walter, Nat Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyze individual cytosine positions, or treated by enzymatic digestion (Xiong & Laird, Nucleic Acids Res., 25:2532-4, 1997). Detection by hybridization has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 9746705 and WO 9515373).

The present invention provides for the use of the bisulfite technique, in combination with one or more methylation assays, for determination of the methylation status of CpG dinucleotide sequences within sequences from the group consisting of SEQ ID NO:1. Preferably said group consists of SEQ ID Nos: 35, 63, 19 and most preferably said sequence is SEQ ID NO: 961 According to the present invention, determination of the methylation status of CpG dinucleotide sequences within sequences from the group consisting of SEQ ID NO:1 and SEQ ID NO: 961 has prognostic utility.

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA. COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or bisulfite treated DNA sequence or CpG island); restriction enzyme and appropriate buffer; gene-hybridization oligo; control hybridization oligo; kinase labeling kit for oligo probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™”, (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with other of these methods.

MethyLight™. The MethyLight™ assay is a high-throughput quantitative methylation assay that utilizes fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or bisulfite treated DNA sequence or CpG island); TaqMan® probes; optimized PCR buffers and deoxynucleotides; and Taq polymerase.

Ms-SNuPE. The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or bisulfite treated DNA sequence or CpG island); optimized PCR buffers and deoxynucleotides; gel extraction kit; positive control primers; Ms-SNuPE primers for specific gene; reaction buffer (for the Ms-SNuPE reaction); and labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or bisulfite treated DNA sequence or CpG island), optimized PCR buffers and deoxynucleotides, and specific probes.

MCA. The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

The Genomic Sequences According to SEQ ID NO: 1 and Non-Naturally Occurring Treated Variants Thereof According to SEQ ID NOS: 2 to 5, were Determined to have Utility for Providing a Prognosis and/or Treatment of Cell Proliferative Disorders, Most Preferably Cancer But not Breast Cancer.

In one embodiment the invention provides a method for providing a prognosis of cell proliferative disorders, most preferably cancer but not breast cancer in a subject. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer. In a particularly preferred embodiment the invention provides a method for the classification based on aggressiveness of a cancer.

Said method comprises the following steps:

i) determining the expression levels of the gene PITX2 and/or regulatory regions thereof; and
ii) determining the prognosis of said cell proliferative disorders.

Said expression level may be determined by any means standard in the art including but not limited to methylation analysis, loss of heterozygosity (hereinafter also referred to as LOH), RNA expression levels and protein expression levels.

Accordingly the present invention also provides prognostic assays and methods, both quantitative and qualitative for detecting the expression of the gene PITX2 in a subject with a cell proliferative disorder, preferably cancer but not breast cancer and determining therefrom upon the prognosis in said subject.

Aberrant expression of mRNA transcribed from the gene PITX2 are associated with prognosis of cancer. Over expression is associated with poor prognosis, under expression is associated with good prognosis.

To detect the presence of mRNA encoding a gene or genomic sequence, a sample is obtained from a patient. The sample may be any suitable sample comprising cellular matter of the tumor, most preferably the primary tumor. Suitable sample types include The DNA source may be any suitable source. Preferably, the source of the DNA sample is selected from the group consisting of cells or cell lines, histological slides, biopsies, paraffin-embedded tissue, bodily fluids, ejaculate, urine, blood, sputum, stool, tissues for example but not limited to those from colon, prostate, lung or liver. and combinations thereof. Preferably, the source is biopsies, bodily fluids, ejaculate, urine, or blood.

In a particularly preferred embodiment of the method said source is primary tumor tissue. The sample may be treated to extract the RNA contained therein. The resulting nucleic acid from the sample is then analyzed. Many techniques are known in the state of the art for determining absolute and relative levels of gene expression, commonly used techniques suitable for use in the present invention include in situ hybridization (e.g. FISH), Northern analysis, RNase protection assays (RPA), microarrays and PCR-based techniques, such as quantitative PCR and differential display PCR or any other nucleic acid detection method.

Particularly preferred is the use of the reverse transcription/polymerization chain reaction technique (RT-PCR). The method of RT-PCR is well known in the art (for example, see Watson and Fleming, supra).

The RT-PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end oligo dT primer and/or random hexamer primers. The cDNA thus produced is then amplified by means of PCR. (Belyavsky et al, Nucl Acid Res 17:2919-2932, 1989; Krug and Berger, Methods in Enzymology, Academic Press, N.Y., Vol. 152, pp. 316-325, 1987 which are incorporated by reference). Further preferred is the “Real-time” variant of RT-PCR, wherein the PCR product is detected by means of hybridization probes (e.g. TaqMan, Lightcycler, Molecular Beacons & Scorpion) or SYBR green. The detected signal from the probes or SYBR green is then quantitated either by reference to a standard curve or by comparing the Ct values to that of a calibration standard. Analysis of housekeeping genes is often used to normalize the results.

In Northern blot analysis total or poly(A)+ mRNA is run on a denaturing agarose gel and detected by hybridization to a labeled probe in the dried gel itself or on a membrane. The resulting signal is proportional to the amount of target RNA in the RNA population.

Comparing the signals from two or more cell populations or tissues reveals relative differences in gene expression levels. Absolute quantitation can be performed by comparing the signal to a standard curve generated using known amounts of an in vitro transcript corresponding to the target RNA. Analysis of housekeeping genes, genes whose expression levels are expected to remain relatively constant regardless of conditions, is often used to normalize the results, eliminating any apparent differences caused by unequal transfer of RNA to the membrane or unequal loading of RNA on the gel.

The first step in Northern analysis is isolating pure, intact RNA from the cells or tissue of interest. Because Northern blots distinguish RNAs by size, sample integrity influences the degree to which a signal is localized in a single band. Partially degraded RNA samples will result in the signal being smeared or distributed over several bands with an overall loss in sensitivity and possibly an erroneous interpretation of the data. In Northern blot analysis, DNA, RNA and oligonucleotide probes can be used and these probes are preferably labeled (e.g. radioactive labels, mass labels or fluorescent labels). The size of the target RNA, not the probe, will determine the size of the detected band, so methods such as random-primed labeling, which generates probes of variable lengths, are suitable for probe synthesis. The specific activity of the probe will determine the level of sensitivity, so it is preferred that probes with high specific activities, are used.

In an RNase protection assay, the RNA target and an RNA probe of a defined length are hybridized in solution. Following hybridization, the RNA is digested with RNases specific for single-stranded nucleic acids to remove any unhybridized, single-stranded target RNA and probe. The RNases are inactivated, and the RNA is separated e.g. by denaturing polyacrylamide gel electrophoresis. The amount of intact RNA probe is proportional to the amount of target RNA in the RNA population. RPA can be used for relative and absolute quantitation of gene expression and also for mapping RNA structure, such as intron/exon boundaries and transcription start sites. The RNase protection assay is preferable to Northern blot analysis as it generally has a lower limit of detection.

The antisense RNA probes used in RPA are generated by in vitro transcription of a DNA template with a defined endpoint and are typically in the range of 50-600 nucleotides. The use of RNA probes that include additional sequences not homologous to the target RNA allows the protected fragment to be distinguished from the full-length probe. RNA probes are typically used instead of DNA probes due to the ease of generating single-stranded RNA probes and the reproducibility and reliability of RNA:RNA duplex digestion with RNases (Ausubel et al., 2003), particularly preferred are probes with high specific activities.

Particularly preferred is the use of microarrays. The microarray analysis process can be divided into two main parts. First is the immobilization of known gene sequences onto glass slides or other solid support followed by hybridization of the fluorescently labeled cDNA (comprising the sequences to be interrogated) to the known genes immobilized on the glass slide. After hybridization, arrays are scanned using a fluorescent microarray scanner. Analyzing the relative fluorescent intensity of different genes provides a measure of the differences in gene expression.

DNA arrays can be generated by immobilizing presynthesized oligonucleotides onto prepared glass slides. In this case, representative gene sequences are manufactured and prepared using standard oligonucleotide synthesis and purification methods. These synthesized gene sequences are complementary to the genes of interest (in this case PITX2) and tend to be shorter sequences in the range of 25-70 nucleotides. Alternatively, immobilized oligos can be chemically synthesized in situ on the surface of the slide. In situ oligonucleotide synthesis involves the consecutive addition of the appropriate nucleotides to the spots on the microarray; spots not receiving a nucleotide are protected during each stage of the process using physical or virtual masks.

In expression profiling microarray experiments, the RNA templates used are representative of the transcription profile of the cells or tissues under study. RNA is first isolated from the cell populations or tissues to be compared. Each RNA sample is then used as a template to generate fluorescently labeled cDNA via a reverse transcription reaction. Fluorescent labeling of the cDNA can be accomplished by either direct labeling or indirect labeling methods. During direct labeling, fluorescently modified nucleotides (e.g., Cy®3- or Cy®₅-dCTP) are incorporated directly into the cDNA during the reverse transcription. Alternatively, indirect labeling can be achieved by incorporating aminoallyl-modified nucleotides during cDNA synthesis and then conjugating an N-hydroxysuccinimide (NHS)-ester dye to the aminoallyl-modified cDNA after the reverse transcription reaction is complete. Alternatively, the probe may be unlabelled, but may be detectable by specific binding with a ligand which is labeled, either directly or indirectly. Suitable labels and methods for labeling ligands (and probes) are known in the art, and include, for example, radioactive labels which may be incorporated by known methods (e.g., nick translation or kinasing). Other suitable labels include but are not limited to biotin, fluorescent groups, chemiluminescent groups (e.g., dioxetanes, particularly triggered dioxetanes), enzymes, antibodies, and the like.

To perform differential gene expression analysis, cDNA generated from different RNA samples are labeled with Cy®3. The resulting labeled cDNA is purified to remove unincorporated nucleotides, free dye and residual RNA. Following purification, the labeled cDNA samples are hybridized to the microarray. The stringency of hybridization is determined by a number of factors during hybridization and during the washing procedure, including temperature, ionic strength, length of time and concentration of formamide. These factors are outlined in, for example, Sambrook et al. (Molecular Cloning: A Laboratory Manual, 2nd ed., 1989). The microarray is scanned post-hybridization using a fluorescent microarray scanner. The fluorescent intensity of each spot indicates the level of expression for that gene; bright spots correspond to strongly expressed genes, while dim spots indicate weak expression.

Once the images are obtained, the raw data must be analyzed. First, the background fluorescence must be subtracted from the fluorescence of each spot. The data is then normalized to a control sequence, such as an exogenously added RNA, or a housekeeping gene panel to account for any nonspecific hybridization, array imperfections or variability in the array setup, cDNA labeling, hybridization or washing. Data normalization allows the results of multiple arrays to be compared.

The present invention further provides for methods for the detection of the presence of the polypeptide encoded by said gene sequences in a sample obtained from a patient.

Aberrant levels of polypeptide expression of the polypeptides encoded by the gene PITX2 are associated with prognosis of cell proliferative disorder, preferably cancer but not breast cancer. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer. Accordingly over or under expression of said polypeptides are associable with the prognosis of cancers. Over expression is associated with poor prognosis and under expression is associated with good prognosis.

Any method known in the art for detecting polypeptides can be used. Such methods include, but are not limited to mass-spectrometry, immunodiffusion, immunoelectrophoresis, immunochemical methods, binder-ligand assays, immunohistochemical techniques, agglutination and complement assays (e.g., see Basic and Clinical Immunology, Sites and Terr, eds., Appleton & Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

Certain embodiments of the present invention comprise the use of antibodies specific to the polypeptide encoded by the PITX2 gene.

Such antibodies are useful for cancer prognostic and/or predictive applications. In certain embodiments production of monoclonal or polyclonal antibodies can be induced by the use of the coded polypeptide as an antigen. Such antibodies may in turn be used to detect expressed polypeptides as markers for cell proliferative disorder, preferably cancer but not breast cancer prognosis. The levels of such polypeptides present may be quantified by conventional methods. Antibody-polypeptide binding may be detected and quantified by a variety of means known in the art, such as labeling with fluorescent or radioactive ligands. The invention further comprises kits for performing the above-mentioned procedures, wherein such kits contain antibodies specific for the investigated polypeptides.

Numerous competitive and non-competitive polypeptide binding immunoassays are well known in the art. Antibodies employed in such assays may be unlabelled, for example as used in agglutination tests, or labeled for use a wide variety of assay methods. Labels that can be used include radionuclides, enzymes, fluorescers, chemiluminescers, enzyme substrates or co-factors, enzyme inhibitors, particles, dyes and the like. Preferred assays include but are not limited to radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art.

In an alternative embodiment of the method the proteins may be detected by means of western blot analysis. Said analysis is standard in the art, briefly proteins are separated by means of electrophoresis e.g. SDS-PAGE. The separated proteins are then transferred to a suitable membrane (or paper) e.g. nitrocellulose, retaining the spatial separation achieved by electrophoresis. The membrane is then incubated with a generic protein (e.g. milk protein) to bind remaining sticky places on the membrane. An antibody specific to the protein of interest is then added, said antibody being detectably labeled for example by dyes or enzymatic means (e.g. alkaline phosphatase or horseradish peroxidase). The location of the antibody on the membrane is then detected.

In an alternative embodiment of the method the proteins may be detected by means of immunohistochemistry (the use of antibodies to probe specific antigens in a sample). Said analysis is standard in the art, wherein detection of antigens in tissues is known as immunohistochemistry, while detection in cultured cells is generally termed immunocytochemistry. Briefly the primary antibody to be detected by binding to its specific antigen. The antibody-antigen complex is then bound by a secondary enzyme conjugated antibody. In the presence of the necessary substrate and chromogen the bound enzyme is detected according to colored deposits at the antibody-antigen binding sites. There is a wide range of suitable sample types, antigen-antibody affinity, antibody types, and detection enhancement methods. Thus optimal conditions for immunohistochemical or immunocytochemical detection must be determined by the person skilled in the art for each individual case.

One approach for preparing antibodies to a polypeptide is the selection and preparation of an amino acid sequence of all or part of the polypeptide, chemically synthesizing the amino acid sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference). Methods for preparation of the polypeptides or epitopes thereof include, but are not limited to chemical synthesis, recombinant DNA techniques or isolation from biological samples.

In the final step of the method the prognosis of the patient is determined, whereby overexpression is indicative of negative prognosis. The term overexpression shall be taken to mean expression at a detected level greater than a pre-determined cut off which may be selected from the group consisting of the mean, median or an optimized threshold value.

Another aspect of the invention provides a kit for use in providing a prognosis of a subject with a cell proliferative disorder, preferably cancer but not breast cancer, comprising: a means for detecting PITX2 polypeptides. The means for detecting the polypeptides comprise preferably antibodies, antibody derivatives, or antibody fragments. The polypeptides are most preferably detected by means of Western blotting utilizing a labeled antibody. In another embodiment of the invention the kit further comprising means for obtaining a biological sample of the patient. Preferred is a kit, which further comprises a container suitable for containing the means for detecting the polypeptides in the biological sample of the patient, and most preferably further comprises instructions for use and interpretation of the kit results. In a preferred embodiment the kit for use in determining treatment strategy for a patient with a cell proliferative disorder, preferably cancer but not breast cancer, comprises: (a) a means for detecting PITX2 polypeptides; (b) a container suitable for containing the said means and the biological sample of the patient comprising the polypeptides wherein the means can form complexes with the polypeptides; (c) a means to detect the complexes of (b); and optionally (d) instructions for use and interpretation of the kit results. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

The kit may also contain other components such as buffers or solutions suitable for blocking, washing or coating, packaged in a separate container.

Another aspect of the invention relates to a kit for use in providing a prognosis of a subject with a cell proliferative disorder, preferably cancer but not breast cancer, said kit comprising: a means for measuring the level of transcription of the gene PITX2. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer. In a preferred embodiment the means for measuring the level of transcription comprise oligonucleotides or polynucleotides able to hybridize under stringent or moderately stringent conditions to the transcription products of PITX2. In a most preferred embodiment the level of transcription is determined by techniques selected from the group of Northern blot analysis, reverse transcriptase PCR, real-time PCR, RNAse protection, and microarray. In another embodiment of the invention the kit further comprises means for obtaining a biological sample of the patient. Preferred is a kit, which further comprises a container suitable for containing the means for measuring the level of transcription and the biological sample of the patient, and most preferably further comprises instructions for use and interpretation of the kit results.

In a preferred embodiment the kit for use in determining treatment strategy for a patient with a cell proliferative disorder, preferably cancer but not breast cancer comprises (a) a plurality of oligonucleotides or polynucleotides able to hybridize under stringent or moderately stringent conditions to the transcription products of the gene PITX2; (b) a container suitable for containing the oligonucleotides or polynucleotides and a biological sample of the patient comprising the transcription products wherein the oligonucleotides or polynucleotide can hybridize under stringent or moderately stringent conditions to the transcription products, (c) means to detect the hybridization of (b); and optionally, (d) instructions for use and interpretation of the kit results.

The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

Most preferably a kit according to the embodiments of the present invention is used for the determination of expression step of the methods according to other aspects of the invention. In a further aspect, the invention provides a further method for providing a prognosis of a subject with a cell proliferative disorder, preferably cancer but not breast cancer comprising the following steps. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer. In the first step of the method a sample is obtained from the subject. Commonly used techniques suitable for use in the present invention include in situ hybridization (e.g. FISH), Northern analysis, RNase protection assays (RPA), microarrays and PCR-based techniques, such as quantitative PCR and differential display PCR or any other nucleic acid detection method.

Particularly preferred is the use of the reverse transcription/polymerization chain reaction technique (RT-PCR). The method of RT-PCR is well known in the art (for example, see Watson and Fleming, supra).

The RT-PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end oligo dT primer and/or random hexamer primers. The cDNA thus produced is then amplified by means of PCR. (Belyavsky et al, Nucl Acid Res 17:2919-2932, 1989; Krug and Berger, Methods in Enzymology, Academic Press, N.Y., Vol. 152, pp. 316-325, 1987 which are incorporated by reference). Further preferred is the “Real-time” variant of RT-PCR, wherein the PCR product is detected by means of hybridization probes (e.g. TaqMan, Lightcycler, Molecular Beacons & Scorpion) or SYBR green. The detected signal from the probes or SYBR green is then quantitated either by reference to a standard curve or by comparing the Ct values to that of a calibration standard. Analysis of housekeeping genes is often used to normalize the results.

In Northern blot analysis total or poly(A)+ mRNA is run on a denaturing agarose gel and detected by hybridization to a labeled probe in the dried gel itself or on a membrane. The resulting signal is proportional to the amount of target RNA in the RNA population.

Comparing the signals from two or more cell populations or tissues reveals relative differences in gene expression levels. Absolute quantitation can be performed by comparing the signal to a standard curve generated using known amounts of an in vitro transcript corresponding to the target RNA. Analysis of housekeeping genes, genes whose expression levels are expected to remain relatively constant regardless of conditions, is often used to normalize the results, eliminating any apparent differences caused by unequal transfer of RNA to the membrane or unequal loading of RNA on the gel.

The first step in Northern analysis is isolating pure, intact RNA from the cells or tissue of interest. Because Northern blots distinguish RNAs by size, sample integrity influences the degree to which a signal is localized in a single band. Partially degraded RNA samples will result in the signal being smeared or distributed over several bands with an overall loss in sensitivity and possibly an erroneous interpretation of the data. In Northern blot analysis, DNA, RNA and oligonucleotide probes can be used and these probes are preferably labeled (e.g. radioactive labels, mass labels or fluorescent labels). The size of the target RNA, not the probe, will determine the size of the detected band, so methods such as random-primed labeling, which generates probes of variable lengths, are suitable for probe synthesis. The specific activity of the probe will determine the level of sensitivity, so it is preferred that probes with high specific activities, are used.

In an RNase protection assay, the RNA target and an RNA probe of a defined length are hybridized in solution. Following hybridization, the RNA is digested with RNases specific for single-stranded nucleic acids to remove any unhybridized, single-stranded target RNA and probe. The RNases are inactivated, and the RNA is separated e.g. by denaturing polyacrylamide gel electrophoresis. The amount of intact RNA probe is proportional to the amount of target RNA in the RNA population. RPA can be used for relative and absolute quantitation of gene expression and also for mapping RNA structure, such as intron/exon boundaries and transcription start sites. The RNase protection assay is preferable to Northern blot analysis as it generally has a lower limit of detection.

The antisense RNA probes used in RPA are generated by in vitro transcription of a DNA template with a defined endpoint and are typically in the range of 50-600 nucleotides. The use of RNA probes that include additional sequences not homologous to the target RNA allows the protected fragment to be distinguished from the full-length probe. RNA probes are typically used instead of DNA probes due to the ease of generating single-stranded RNA probes and the reproducibility and reliability of RNA:RNA duplex digestion with RNases (Ausubel et al. 2003), particularly preferred are probes with high specific activities.

Particularly preferred is the use of microarrays. The microarray analysis process can be divided into two main parts. First is the immobilization of known gene sequences onto glass slides or other solid support followed by hybridization of the fluorescently labelled cDNA (comprising the sequences to be interrogated) to the known genes immobilized on the glass slide. After hybridization, arrays are scanned using a fluorescent microarray scanner. Analyzing the relative fluorescent intensity of different genes provides a measure of the differences in gene expression.

DNA arrays can be generated by immobilizing presynthesized oligonucleotides onto prepared glass slides. In this case, representative gene sequences are manufactured and prepared using standard oligonucleotide synthesis and purification methods. These synthesized gene sequences are complementary to the genes of interest (in this case PITX2) and tend to be shorter sequences in the range of 25-70 nucleotides. Alternatively, immobilized oligos can be chemically synthesized in situ on the surface of the slide. In situ oligonucleotide synthesis involves the consecutive addition of the appropriate nucleotides to the spots on the microarray; spots not receiving a nucleotide are protected during each stage of the process using physical or virtual masks.

In expression profiling microarray experiments, the RNA templates used are representative of the transcription profile of the cells or tissues under study. RNA is first isolated from the cell populations or tissues to be compared. Each RNA sample is then used as a template to generate fluorescently labelled cDNA via a reverse transcription reaction. Fluorescent labeling of the cDNA can be accomplished by either direct labeling or indirect labeling methods. During direct labeling, fluorescently modified nucleotides (e.g., Cy®3- or Cy®5-dCTP) are incorporated directly into the cDNA during the reverse transcription. Alternatively, indirect labeling can be achieved by incorporating aminoallyl-modified nucleotides during cDNA synthesis and then conjugating an N-hydroxysuccinimide (NHS)-ester dye to the aminoallyl-modified cDNA after the reverse transcription reaction is complete. Alternatively, the probe may be unlabelled, but may be detectable by specific binding with a ligand which is labelled, either directly or indirectly. Suitable labels and methods for labeling ligands (and probes) are known in the art, and include, for example, radioactive labels which may be incorporated by known methods (e.g., nick translation or kinasing). Other suitable labels include but are not limited to biotin, fluorescent groups, chemiluminescent groups (e.g., dioxetanes, particularly triggered dioxetanes), enzymes, antibodies, and the like.

To perform differential gene expression analysis, cDNA generated from different RNA samples are labelled with Cy®3. The resulting labelled cDNA is purified to remove unincorporated nucleotides, free dye and residual RNA. Following purification, the labeled cDNA samples are hybridized to the microarray. The stringency of hybridization is determined by a number of factors during hybridization and during the washing procedure, including temperature, ionic strength, length of time and concentration of formamide. These factors are outlined in, for example, Sambrook et al. (Molecular Cloning: A Laboratory Manual, 2nd ed., 1989). The microarray is scanned post-hybridization using a fluorescent microarray scanner. The fluorescent intensity of each spot indicates the level of expression for that gene; bright spots correspond to strongly expressed genes, while dim spots indicate weak expression.

Once the images are obtained, the raw data must be analyzed. First, the background fluorescence must be subtracted from the fluorescence of each spot. The data is then normalized to a control sequence, such as an exogenously added RNA, or a housekeeping gene panel to account for any nonspecific hybridization, array imperfections or variability in the array setup, cDNA labeling, hybridization or washing. Data normalization allows the results of multiple arrays to be compared.

The present invention further provides for methods for the detection of the presence of the polypeptide encoded by said gene sequences in a sample obtained from a patient.

Aberrant levels of polypeptide expression of the polypeptides encoded by the gene PITX2 are associated with cell proliferative disorder, preferably cancer but not breast cancer prognosis and/or treatment outcome. It is particularly preferred that said cancers are selected from the group consisting bladder cancer, colorectal cancer, endometrial cancer, kidney (renal cell) cancer, leukemia, lung (Including bronchus) cancer, melanoma, non-Hodgkin's lymphoma, pancreatic cancer, prostate cancer, skin cancer and thyroid cancer.

Accordingly over or under expression of said polypeptides are associable with the prognosis and to treatment outcome of cancers. Over expression is associated with poor prognosis and under expression is associated with good prognosis.

Any method known in the art for detecting polypeptides can be used. Such methods include, but are not limited to mass-spectrometry, immunodiffusion, immunoelectrophoresis, immunochemical methods, binder-ligand assays, immunohistochemical techniques, agglutination and complement assays (e.g., see Basic and Clinical Immunology, Sites and Terr, eds., Appleton & Lange, Norwalk, Conn. pp 217-262, 1991 which is incorporated by reference). Preferred are binder-ligand immunoassay methods including reacting antibodies with an epitope or epitopes and competitively displacing a labelled polypeptide or derivative thereof.

Certain embodiments of the present invention comprise the use of antibodies specific to the polypeptide encoded by the PITX2 gene.

Such antibodies are useful for cancer prognostic and/or predictive applications. In certain embodiments production of monoclonal or polyclonal antibodies can be induced by the use of the coded polypeptide as an antigen. Such antibodies may in turn be used to detect expressed polypeptides as markers for cell proliferative disorder, preferably cancer but not breast cancer prognosis. The levels of such polypeptides present may be quantified by conventional methods. Antibody-polypeptide binding may be detected and quantified by a variety of means known in the art, such as labeling with fluorescent or radioactive ligands. The invention further comprises kits for performing the above-mentioned procedures, wherein such kits contain antibodies specific for the investigated polypeptides.

Numerous competitive and non-competitive polypeptide binding immunoassays are well known in the art. Antibodies employed in such assays may be unlabelled, for example as used in agglutination tests, or labelled for use a wide variety of assay methods. Labels that can be used include radionuclides, enzymes, fluorescers, chemiluminescers, enzyme substrates or co-factors, enzyme inhibitors, particles, dyes and the like. Preferred assays include but are not limited to radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art.

In an alternative embodiment of the method the proteins may be detected by means of western blot analysis. Said analysis is standard in the art, briefly proteins are separated by means of electrophoresis e.g. SDS-PAGE. The separated proteins are then transferred to a suitable membrane (or paper) e.g. nitrocellulose, retaining the spatial separation achieved by electrophoresis. The membrane is then incubated with a generic protein (e.g. milk protein) to bind remaining sticky places on the membrane. An antibody specific to the protein of interest is then added, said antibody being detectably labelled for example by dyes or enzymatic means (e.g. alkaline phosphatase or horseradish peroxidase). The location of the antibody on the membrane is then detected.

In an alternative embodiment of the method the proteins may be detected by means of immunohistochemistry (the use of antibodies to probe specific antigens in a sample). Said analysis is standard in the art, wherein detection of antigens in tissues is known as immunohistochemistry, while detection in cultured cells is generally termed immunocytochemistry. Briefly the primary antibody to be detected by binding to its specific antigen. The antibody-antigen complex is then bound by a secondary enzyme conjugated antibody. In the presence of the necessary substrate and chromogen the bound enzyme is detected according to colored deposits at the antibody-antigen binding sites. There is a wide range of suitable sample types, antigen-antibody affinity, antibody types, and detection enhancement methods. Thus optimal conditions for immunohistochemical or immunocytochemical detection must be determined by the person skilled in the art for each individual case.

One approach for preparing antibodies to a polypeptide is the selection and preparation of an amino acid sequence of all or part of the polypeptide, chemically synthesizing the amino acid sequence and injecting it into an appropriate animal, usually a rabbit or a mouse (Milstein and Kohler Nature 256:495-497, 1975; Gulfre and Milstein, Methods in Enzymology: Immunochemical Techniques 73:146, Langone and Banatis eds., Academic Press, 1981 which are incorporated by reference). Methods for preparation of the polypeptides or epitopes thereof include, but are not limited to chemical synthesis, recombinant DNA techniques or isolation from biological samples.

In a particularly preferred embodiment the expression level of the gene PITX2 is determined by analysis of the level of methylation of said gene and/or regulatory regions thereof. It is preferred that the level of methylation of said gene and/or regulatory regions thereof is determined by determining the methylation status or level of at least one CpG dinucleotide thereof. It is further preferred that the level of methylation of said gene and/or regulatory regions thereof is determined by determining the methylation status or level of a plurality of CpG dinucleotides thereof. Said analysis comprises the following steps:

i) contacting genomic DNA obtained from the subject with at least one reagent, or series of reagents that distinguishes between methylated and non-methylated CpG dinucleotides within at least one target region of the genomic DNA, wherein said contiguous nucleotides comprise at least one CpG dinucleotide sequence; and
ii) classifying the cell proliferative disorder, (most preferably cancer but not breast cancer) according to its prognosis as determined from the methylation status of said target regions analyzed in i).

Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. Preferably, the source of the DNA sample is selected from the group consisting of cells or cell lines, histological slides, biopsies, paraffin-embedded tissue, bodily fluids, ejaculate, urine, blood, and combinations thereof. Preferably, the source is biopsies, bodily fluids, ejaculate, urine, or blood. The genomic DNA sample is then treated in such a manner that cytosine bases which are unmethylated at the 5′-position are converted to uracil, thymine, or another base which is dissimilar to cytosine in terms of hybridization behavior. This will be understood as ‘treatment’ herein.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

In a preferred embodiment said method is achieved by contacting the nucleic acid of the gene PITX2 and/or its regulatory regions, or sequences thereof according to SEQ ID NO: 1 in a biological sample obtained from a subject with at least one reagent or a series of reagents, wherein said reagent or series of reagents, distinguishes between methylated and non methylated CpG dinucleotides within the target nucleic acid.

In a preferred embodiment, the method comprises the following steps: Preferably, said method comprises the following steps: In the first step, a sample of the tissue to be analyzed is obtained. The source may be any suitable source, such as The DNA source may be any suitable source. Preferably, the source of the DNA sample is selected from the group consisting of cells or cell lines, histological slides, biopsies, paraffin-embedded tissue, bodily fluids, ejaculate, urine, blood, sputum, stool, tissues for example but not limited to those from colon, prostate, lung or liver, and combinations thereof. Preferably, the source is biopsies, bodily fluids, ejaculate, urine, or blood. The DNA is then isolated from the sample. Extraction may be by means that are standard to one skilled in the art, including the use of commercially available kits, detergent lysates, sonification and vortexing with glass beads. Briefly, wherein the DNA of interest is encapsulated by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ herein.

This is preferably achieved by means of treatment with a bisulfite reagent. The term “bisulfite reagent” refers to a reagent comprising bisulfite, disulfite, hydrogen sulfite or combinations thereof, useful as disclosed herein to distinguish between methylated and unmethylated CpG dinucleotide sequences. Methods of said treatment are known in the art (e.g. PCT/EP2004/011715, which is incorporated by reference in its entirety). It is preferred that the bisulfite treatment is conducted in the presence of denaturing solvents such as but not limited to n-alkylenglycol, particularly diethylene glycol dimethyl ether (DME), or in the presence of dioxane or dioxane derivatives. In a preferred embodiment the denaturing solvents are used in concentrations between 1% and 35% (v/v). It is also preferred that the bisulfite reaction is carried out in the presence of scavengers such as but not limited to chromane derivatives, e.g., 6-hydroxy-2,5,7,8,-tetramethylchromane 2-carboxylic acid (see: PCT/EP2004/011715 which is incorporated by reference in its entirety). The bisulfite conversion is preferably carried out at a reaction temperature between 30° C. and 70° C., whereby the temperature is increased to over 85° C. for short periods of times during the reaction (see: PCT/EP2004/011715 which is incorporated by reference in its entirety). The bisulfite treated DNA is preferably purified prior to the quantification. This may be conducted by any means known in the art, such as but not limited to ultrafiltration, preferably carried out by means of Microcon™columns (manufactured by Millipore™). The purification is carried out according to a modified manufacturer's protocol (see: PCT/EP2004/011715 which is incorporated by reference in its entirety).

In the third step of the method, fragments of the pretreated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary to, identical to, or hybridize under stringent or highly stringent conditions to an at least 16-base-pair long segment of the base sequences of one of SEQ ID NO: 2 to SEQ ID NO: 5 and sequences complementary thereto.

In an alternate embodiment of the method, the methylation status of preselected CpG positions within SEQ ID NO: 1, may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer that hybridizes to a bisulfite treated CpG dinucleotide. Therefore, the sequence of said primers comprises at least one CpG or TpG dinucleotide. MSP primers specific for non-methylated DNA contain a ‘T’ at the 3′ position of the C position in the CpG. Preferably, therefore, the base sequence of said primers is required to comprise a sequence having a length of at least 9 nucleotides which hybridizes to a pretreated nucleic acid sequence according to one of SEQ ID NO: 2 to SEQ ID NO: 5 and sequences complementary thereto, wherein the base sequence of said oligomers comprises at least one CpG dinucleotide.

A further preferred embodiment of the method comprises the use of blocker oligonucleotides. The use of such blocker oligonucleotides has been described by Yu et al., BioTechniques 23:714-720, 1997. Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpG’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-termini thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase. Preferably, therefore, the base sequence of said blocking oligonucleotides is required to comprise a sequence having a length of at least 9 nucleotides which hybridizes to a pretreated nucleic acid sequence according to one of SEQ ID NO: 2 to SEQ ID NO: 5, and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass that can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas and Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut and Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallization. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut and Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analyzed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesized in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to SEQ ID NO: 1, and the equivalent positions within SEQ ID NO: 2 TO SEQ ID NO: 5. Said oligonucleotides may also be present in the form of peptide nucleic acids. The non-hybridized amplificates are then removed. The hybridized amplificates are then detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridized to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which, in preferred embodiments, is designed to hybridize to a GpC-rich sequence located between the forward and reverse amplification primers. The TaqMan™ probe further comprises a fluorescent reporter moiety and a quencher moiety covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight assay. Variations on the TaqMan™ detection methodology that are also suitable for use with the described invention include the use of dual-probe technology (Lightcycler) or fluorescent amplification primers (Sunrise technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids In a further preferred embodiment of the method, the fifth step of the method comprises the use of template-directed oligonucleotide extension, such as MS-SNuPE as described by Gonzalgo and Jones, Nucleic Acids Res. 25:2529-2531, 1997.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

In one preferred embodiment of the method the nucleic acid according to SEQ ID NO: 1, are isolated and treated according to the first three steps of the method outlined above, namely:

a) obtaining, from a subject, a biological sample having subject genomic DNA;
b) extracting or otherwise isolating the genomic DNA; and
c) treating the genomic DNA of b), or a fragment thereof, with one or more reagents to convert cytosine bases that are unmethylated in the 5-position thereof to uracil or to another base that is detectably dissimilar to cytosine in terms of hybridization properties;
and wherein the subsequent amplification of d) is carried out in a methylation specific manner, namely by use of methylation specific primers or blocking oligonucleotides, and further wherein the detection of the amplificates is carried out by means of a real-time detection probes, as described above.

Wherein the subsequent amplification of d) is carried out by means of methylation specific primers, as described above, said methylation specific primers comprise a sequence having a length of at least 9 nucleotides which hybridizes to a pretreated nucleic acid sequence according to one of SEQ ID NO: 2 to SEQ ID NO: 5, and sequences complementary thereto, wherein the base sequence of said oligomers comprises at least one CpG dinucleotide.

Step e) of the method, namely the detection of the specific amplificates indicative of the methylation status of one or more CpG positions according to SEQ ID NO: 1 is carried out by means of real-time detection methods as described above.

In an alternative most preferred embodiment of the method the subsequent amplification of d) is carried out in the presence of blocking oligonucleotides, as described above. Said blocking oligonucleotides comprising a sequence having a length of at least 9 nucleotides which hybridizes to a pretreated nucleic acid sequence according to one of SEQ ID NO: 2 to SEQ ID NO: 5 and sequences complementary thereto, wherein the base sequence of said oligomers comprises at least one CpG, TpG or CpA dinucleotide. Step e) of the method, namely the detection of the specific amplificates indicative of the methylation status of one or more CpG positions according to SEQ ID NO: 1 is carried out by means of real-time detection methods as described above.

In a further preferred embodiment of the method the nucleic acids according to SEQ ID NO: 1 are isolated and treated according to the first three steps of the method outlined above, namely:

a) obtaining, from a subject, a biological sample having subject genomic DNA;
b) extracting or otherwise isolating the genomic DNA;
c) treating the genomic DNA of b), or a fragment thereof, with one or more reagents to convert cytosine bases that are unmethylated in the 5-position thereof to uracil or to another base that is detectably dissimilar to cytosine in terms of hybridization properties; and wherein
d) amplifying subsequent to treatment in c) is carried out in a methylation specific manner, namely by use of methylation specific primers or blocking oligonucleotides, and further wherein
e) detecting of the amplificates is carried out by means of a real-time detection probes, as described above.

Wherein the subsequent amplification of c) is carried out by means of methylation specific primers, as described above, said methylation specific primers comprise a sequence having a length of at least 9 nucleotides which hybridizes to a pretreated nucleic acid sequence according to one of SEQ ID NO: 2 to SEQ ID NO: 5 and sequences complementary thereto, wherein the base sequence of said oligomers comprises at least one CpG dinucleotide.

Additional embodiments of the invention provide a method for the analysis of the methylation status of genomic DNA according to the invention (SEQ ID NO: 1, and the complement thereof) without the need for pretreatment.

In the first step of such additional embodiments, the genomic DNA sample is isolated from tissue or cellular sources. Preferably, such sources include cell lines, histological slides, paraffin embedded tissues, body fluids, or tissue embedded in paraffin. In the second step, the genomic DNA is extracted. Extraction may be by means that are standard to one skilled in the art, including but not limited to the use of detergent lysates, sonification and vortexing with glass beads. Once the nucleic acids have been extracted, the genomic double-stranded DNA is used in the analysis.

In a preferred embodiment, the DNA may be cleaved prior to the treatment, and this may be by any means standard in the state of the art, in particular with methylation-sensitive restriction endonucleases.

In the third step, the DNA is then digested with one or more methylation sensitive restriction enzymes. The digestion is carried out such that hydrolysis of the DNA at the restriction site is informative of the methylation status of a specific CpG dinucleotide.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionucleotides and mass labels.

In the fifth step the amplificates are detected. The detection may be by any means standard in the art, for example, but not limited to, gel electrophoresis analysis, hybridization analysis, incorporation of detectable tags within the PCR products, DNA array analysis, MALDI or ESI analysis.

In the final step of the method the prognosis of the patient is determined. Hypermethylation and over expression of the gene PITX2 and/or genomic sequences thereof according to SEQ ID NO: 1 are associated with negative prognosis. Patients with predicted positive outcome (i.e. hypomethylation or under expression) after said treatment will accordingly have a decreased absolute reduction of risk of recurrence and death after treatment with primary or adjuvant treatment. Patients with predicted negative outcome (i.e. hypermethylation or over expression) after said treatment will accordingly have a relatively larger absolute reduction of risk of recurrence and death after said treatment. Accordingly patients with a negative outcome will be considered more suitable candidates for aggressive treatment such as chemotherapy or other adjuvant therapies than patients with a positive outcome. Patients with a positive outcome may accordingly be prevented from over prescription of e.g. chemotherapeutic treatment.

The present invention provides novel uses for the genomic sequence of SEQ ID NO:1. Additional embodiments provide modified variants of SEQ ID NO:1, as well as oligonucleotides and/or PNA-oligomers for analysis of cytosine methylation patterns of SEQ ID NO: 1.

An objective of the invention comprises analysis of the methylation state of one or more CpG dinucleotides of the gene PITX2, preferably of SEQ ID NO: 1.

The disclosed invention provides treated nucleic acids, derived from genomic SEQ ID NO:1, wherein the treatment is suitable to convert at least one unmethylated cytosine base of the genomic DNA sequence to uracil or another base that is detectably dissimilar to cytosine in terms of hybridization. The genomic sequences in question may comprise one, or more, consecutive or random methylated CpG positions. Said treatment preferably comprises use of a reagent selected from the group consisting of bisulfite, hydrogen sulfite, disulfite, and combinations thereof. In a preferred embodiment of the invention, the objective comprises analysis of a non-naturally occurring modified nucleic acid comprising a sequence of at least 16 contiguous nucleotide bases in length of a sequence selected from the group consisting of SEQ ID NO: 2 TO SEQ ID NO: 5. Particularly preferred is a non-naturally occurring modified nucleic acid comprising a sequence of at least 16 contiguous nucleotide bases in length of a sequence selected from the group consisting of SEQ ID NO: 65 to SEQ ID NO: 320 and SEQ ID NO: 962 to SEQ ID NO: 965 that is not identical to or complementary to SEQ ID NO: 1 to SEQ ID NO: 64 and SEQ ID NO: 961 or other human genomic DNA. Further preferred is a non-naturally occurring modified nucleic acid comprising a sequence of at least 16 contiguous nucleotide bases in length of a sequence selected from the group consisting of SEQ ID Nos: 133, 134, 261, 262, 189, 190, 317, 318, 101, 102, 229, 230, 962-965 that is not identical to or complementary to SEQ ID Nos: 961, 35, 63 and 19 or other human genomic DNA.

It is further preferred that said sequence comprises at least one CpG, TpA or CpA dinucleotide and sequences complementary thereto. The sequences of SEQ ID NO: 2 TO SEQ ID NO: 5 provide non-naturally occurring modified versions of the nucleic acid according to SEQ ID NO:1, wherein the modification of each genomic sequence results in the synthesis of a nucleic acid having a sequence that is unique and distinct from said genomic sequence as follows. For each sense strand genomic DNA, e.g., SEQ ID NO:1, four converted versions are disclosed. A first version wherein “C” is converted to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” is converted to “T,” but “CpG” remains “CpG” (i.e., corresponds to case where, for all “C” residues of CpG dinucleotide sequences are methylated and are thus not converted). The ‘upmethylated’ converted sequence of SEQ ID NO:1 corresponds to SEQ ID NO:2 to SEQ ID NO:3. A third chemically converted version of each genomic sequences is provided, wherein “C” is converted to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” is converted to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO:1 correspond to SEQ ID NO: 4 to SEQ ID NO: 5.

In an alternative preferred embodiment, such analysis comprises the use of an oligonucleotide or oligomer for detecting the cytosine methylation state within genomic or treated (chemically modified) DNA, according to SEQ ID NO:1 to SEQ ID NO: 5. Said oligonucleotide or oligomer comprising a nucleic acid sequence having a length of at least nine (9) nucleotides which hybridizes, under moderately stringent or stringent conditions (as defined herein above), to a treated nucleic acid sequence according to SEQ ID NO:2 to SEQ ID NO: 5 and/or sequences complementary thereto, or to a genomic sequence according to SEQ ID NO:1 and/or sequences complementary thereto.

Thus, the present invention includes nucleic acid molecules (e.g., oligonucleotides and peptide nucleic acid (PNA) molecules (PNA-oligomers)) that hybridize under moderately stringent and/or stringent hybridization conditions to all or a portion of the sequences SEQ ID NO: 1 to SEQ ID NO: 5, or to the complements thereof. Particularly preferred is a nucleic acid molecule that hybridizes under moderately stringent and/or stringent hybridization conditions to all or a portion of the sequences SEQ ID NO: 2 to SEQ ID NO: 5 but is not identical to or complementary to SEQ ID NO: 1 or other human genomic DNA. Further preferred is a nucleic acid molecule that hybridizes under moderately stringent and/or stringent hybridization conditions to all or a portion of the sequences SEQ ID NO: 2 to SEQ ID NO: 5 but is not identical to or complementary to SEQ ID NO: 1 or other human genomic DNA.

The hybridizing portion of the hybridizing nucleic acids is typically at least 9, 15, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NO: 1 to SEQ ID NO: 5, or to the complements thereof.

Hybridizing nucleic acids of the type described herein can be used, for example, as a primer (e.g., a PCR primer), or a diagnostic and/or prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

Hybridizing nucleic acids of the type described herein can be used, for example, as a primer (e.g., a PCR primer), or a prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO:1 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO:1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));
where n=1, 2, 3, . . . (Y−(X−1));
where Y equals the length (nucleotides or base pairs) of SEQ ID NO: 1 (28536);
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO of length Y is equal to Y−(X−1). For example Z=28536−19=28517 for either sense or antisense sets of SEQ ID NO:1, where X=20.

Preferably, the set is limited to those oligomers that comprise at least one CpG, TpG or CpA dinucleotide.

Examples of inventive 20-mer oligonucleotides include the following set of oligomers (and the antisense set complementary thereto), indicated by polynucleotide positions with reference to SEQ ID NO: 1: 1-20, 2-21, 3-22, 4-23, 5-24 . . . 28517-28536.

Preferably, the set is limited to those oligomers that comprise at least one CpG, TpG or CpA dinucleotide.

Likewise, examples of inventive 25-mer oligonucleotides include the following set of 2,256 oligomers (and the antisense set complementary thereto), indicated by polynucleotide positions with reference to SEQ ID NO:1:

1-25, 2-26, 3-27, 4-28, 5-29 . . . 28512-28536.

Preferably, the set is limited to those oligomers that comprise at least one CpG, TpG or CpA dinucleotide.

The present invention encompasses, for SEQ ID NO:1 to SEQ ID NO: 5 multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO:1.

Preferred sets of such oligonucleotides or modified oligonucleotides of length X are those consecutively overlapping sets of oligomers corresponding to SEQ ID NO:1 to SEQ ID NO:5 (and to the complements thereof). Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinucleotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophores, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of at least one of the CpG dinucleotides of genomic sequences SEQ ID NO:1 and sequences complementary thereto, or to the corresponding CpG, TpG or CpA dinucleotide within a sequence of the treated nucleic acids according to SEQ ID NO: 2 to SEQ ID NO: 5 and sequences complementary thereto. However, it is anticipated that for economic or other factors it may be preferable to analyze a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state in treated genomic DNA (SEQ ID NO: 2 to SEQ ID NO:5), or in genomic DNA (SEQ ID NO:1 and sequences complementary thereto). These probes enable diagnosis and/or classification of genetic and epigenetic parameters of cell proliferative disorders, most preferably cancer but not breast cancer. The set of oligomers may also be used for detecting single nucleotide polymorphisms (SNPs) in treated genomic DNA (SEQ ID NO: 2 to SEQ ID NO: 5), or in genomic DNA (SEQ ID NO:1 and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO:1 to SEQ ID NO:5 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminum, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilized for at least one of: prognosis of; treatment of; monitoring of; and treatment and monitoring of cell proliferative disorders, most preferably cancer but not breast cancer. This is enabled by use of said sets for providing a prognosis of a biological sample isolated from a patient. Particularly preferred are those sets of oligomer that comprise at least two oligonucleotides selected from one of the following sets of oligonucleotides.

In one embodiment of the method, this is achieved by analysis of the methylation status of at least one target sequence comprising, or hybridizing under stringent conditions to at least 16 contiguous nucleotides of the gene PITX2 and/or regulatory regions thereof.

The present invention further provides a method for ascertaining genetic and/or epigenetic parameters of the genomic sequences according to SEQ ID NO:1 within a subject by analyzing cytosine methylation and single nucleotide polymorphisms. In a preferred embodiment the present invention further provides a method for ascertaining genetic and/or epigenetic parameters of the genomic sequences according to SEQ ID NO:1 within a subject by analyzing cytosine methylation and single nucleotide polymorphisms. Said method comprising contacting a nucleic acid comprising SEQ ID NO:1 in a biological sample obtained from said subject with at least one reagent or a series of reagents, wherein said reagent or series of reagents, distinguishes between methylated and non-methylated CpG dinucleotides within the target nucleic acid.

Preferably, said method comprises the following steps: In the first step, a sample of the tissue to be analyzed is obtained. The source may be any suitable source. Preferably, the source of the DNA sample is selected from the group consisting of cells or cell lines, histological slides, biopsies, paraffin-embedded tissue, bodily fluids, ejaculate, urine, blood, and combinations thereof. Preferably, the source is biopsies, bodily fluids, ejaculate, urine, or blood.

The genomic DNA is then isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ or ‘treatment’ herein.

The above-described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

In the third step of the method, fragments of the treated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary, identical, or hybridize under stringent or highly stringent conditions to an at least 16-base-pair long segment of the base sequences of one of SEQ ID NO: 2 to SEQ ID NO: 5 (preferably one of SEQ ID Nos: 133, 134, 261, 262, 189, 190, 317, 318, 101, 102, 229, 230 and most preferably one of SEQ ID Nos: 962-965) and sequences complementary thereto.

In an alternate embodiment of the method, the methylation status of preselected CpG positions within the nucleic acid sequences comprising one or more of SEQ ID NO:1 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridizes to a bisulfite treated CpG dinucleotide. Therefore, the sequence of said primers comprises at least one CpG dinucleotide. MSP primers specific for non-methylated DNA contain a “T” at the position of the C position in the CpG. Preferably, therefore, the base sequence of said primers is required to comprise a sequence having a length of at least 9 nucleotides which hybridizes to a treated nucleic acid sequence according to one of SEQ ID NO:2 to SEQ ID NO:5 and sequences complementary thereto, wherein the base sequence of said oligomers comprises at least one CpG dinucleotide.

A further preferred embodiment of the method comprises the use of blocker oligonucleotides. The use of such blocker oligonucleotides has been described by Yu et al., BioTechniques 23:714-720, 1997. Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridizes to a treated nucleic acid sequence according to one of SEQ ID NO: 2 to SEQ ID NO: 5 and sequences complementary thereto, wherein the base sequence of said oligonucleotides comprises at least one CpG, TpG or CpA dinucleotide. More preferably the base sequence of said blocking oligonucleotides is required to comprise a sequence having a length of at least 9 nucleotides which hybridizes to a treated nucleic acid sequence according to one of preferably SEQ ID NO:2 to SEQ ID NO:5 and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionately with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallization. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analyzed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesized in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to SEQ ID NO:1, and the equivalent positions within SEQ ID NO: 2 to SEQ ID NO: 5. Said oligonucleotides may also be present in the form of peptide nucleic acids. The non-hybridized amplificates are then removed. The hybridized amplificates are then detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridized to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which, in preferred embodiments, is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids In a further preferred embodiment of the method, the fifth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

In the most preferred embodiment of the method the genomic nucleic acids are isolated and treated according to the first three steps of the method outlined above, namely:

a) obtaining, from a subject, a biological sample having subject genomic DNA;
b) extracting or otherwise isolating the genomic DNA;
c) treating the genomic DNA of b), or a fragment thereof, with one or more reagents to convert cytosine bases that are unmethylated in the 5-position thereof to uracil or to another base that is detectably dissimilar to cytosine in terms of hybridization properties; and wherein
d) amplifying subsequent to treatment in c) is carried out in a methylation specific manner, namely by use of methylation specific primers or blocking oligonucleotides, and further wherein
e) detecting of the amplificates is carried out by means of a real-time detection probe, as described above.

Preferably, where the subsequent amplification of d) is carried out by means of methylation specific primers, as described above, said methylation specific primers comprise a sequence having a length of at least 9 nucleotides which hybridizes to a treated nucleic acid sequence according to one of SEQ ID NO: 2 to SEQ ID NO: 5 and sequences complementary thereto, wherein the base sequence of said oligomers comprises at least one CpG dinucleotide. More preferably, where the subsequent amplification of d) is carried out by means of methylation specific primers, as described above, said methylation specific primers comprise a sequence having a length of at least 9 nucleotides which hybridizes to a treated nucleic acid sequence according to one of SEQ ID NO:2 to SEQ ID NO:5 and sequences complementary thereto, wherein the base sequence of said oligomers comprises at least one CpG dinucleotide.

In an alternative most preferred embodiment of the method, the subsequent amplification of d) is carried out in the presence of blocking oligonucleotides, as described above. Said blocking oligonucleotides comprising a sequence having a length of at least 9 nucleotides which hybridizes to a treated nucleic acid sequence according to one of SEQ ID NO:2 to SEQ ID NO:5 and sequences complementary thereto, wherein the base sequence of said oligomers comprises at least one CpG, TpG or CpA dinucleotide. Preferably said blocking oligonucleotides comprising a sequence having a length of at least 9 nucleotides which hybridizes to a treated nucleic acid sequence according to one of SEQ ID NO:2 to SEQ ID NO:5 and sequences complementary thereto, wherein the base sequence of said oligomers comprises at least one CpG, TpG or CpA dinucleotide.

Step e) of the method, namely the detection of the specific amplificates indicative of the methylation status of one or more CpG positions according to SEQ ID NO:1 is carried out by means of real-time detection methods as described above.

Additional embodiments of the invention provide a method for the analysis of the methylation status of genomic DNA according to the invention SEQ ID NO: 1 and complements thereof without the need for pretreatment.

In the first step of such additional embodiments, the genomic DNA sample is isolated from tissue or cellular sources. Preferably, such sources include cell lines, histological slides, body fluids, or tissue embedded in paraffin. In the second step, the genomic DNA is extracted. Extraction may be by means that are standard to one skilled in the art, including but not limited to the use of detergent lysates, sonification and vortexing with glass beads. Once the nucleic acids have been extracted, the genomic double-stranded DNA is used in the analysis.

In a preferred embodiment, the DNA may be cleaved prior to the treatment, and this may be by any means standard in the state of the art, in particular with methylation-sensitive restriction endonucleases.

In the third step, the DNA is then digested with one or more methylation sensitive restriction enzymes. The digestion is carried out such that hydrolysis of the DNA at the restriction site is informative of the methylation status of a specific CpG dinucleotide.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels.

In the fifth step the amplificates are detected. The detection may be by any means standard in the art, for example, but not limited to, gel electrophoresis analysis, hybridization analysis, incorporation of detectable tags within the PCR products, DNA array analysis, MALDI or ESI analysis.

Subsequent to the determination of the methylation state of the genomic nucleic acids the prognosis of the cancer is deduced based upon the methylation state of at least one CpG dinucleotide sequence of SEQ ID NO:1, or an average, or a value reflecting an average methylation state of a plurality of CpG dinucleotide sequences of SEQ ID NO:1. Preferably said prognosis is based upon the methylation state of at least one CpG dinucleotide sequence of the gene PITX2, or an average, or a value reflecting an average methylation state of a plurality of CpG dinucleotide sequences of SEQ ID NO: 1. Hypermethylation of said CpG positions are associated with good prognosis, and hypomethylation is associated with poor prognosis. The cut-off point for determining said hypo and hyper methylation is may be the median methylation level for a given population, or is preferably an optimized cut-off level. For the analysis of PITX2 it is preferred that the cut-off is between 20% and 10% methylation, and most preferably 14.27%. Wherein the methods according to the present invention of expression analysis (most preferably by means of methylation analysis), of the herein described marker are used to determine the prognosis of a cancer patient said methods are preferably used in combination with other clinical prognostic variables used to determine prognosis (i.e. that said variables are factored in or taken into account).

### Kits

Moreover, an additional aspect of the present invention is a kit comprising, for example: a bisulfite-containing reagent; a set of primer oligonucleotides containing at least two oligonucleotides whose sequences in each case correspond, are complementary, or hybridize under stringent or highly stringent conditions to a 16-base long segment of the sequences SEQ ID NO: 1 to SEQ ID NO: 5; oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing. However, a kit along the lines of the present invention can also contain only part of the aforementioned components.

Preferably said kit comprises a bisulfite-containing reagent; a set of primer oligonucleotides containing at least two oligonucleotides whose sequences in each case correspond, are complementary, or hybridize under stringent or highly stringent conditions to a 16-base long segment of the sequences SEQ ID NO:2 to SEQ ID NO:5; oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing. However, a kit along the lines of the present invention can also contain only part of the aforementioned components.

The described invention further provides a composition of matter useful for providing a prognosis of cancer patients. Said composition comprising at least one nucleic acid 18 base pairs in length of a segment of a nucleic acid sequence selected from the group consisting SEQ ID NO:2 to SEQ ID NO:5, and one or more substances taken from the group comprising: magnesium chloride, dNTP, Taq polymerase, bovine serum albumen, an oligomer in particular an oligonucleotide or peptide nucleic acid (PNA)-oligomer, said oligomer comprising in each case at least one base sequence having a length of at least 9 nucleotides which is complementary to, or hybridizes under moderately stringent or stringent conditions to a pretreated genomic DNA according to one of the SEQ ID NO:2 to SEQ ID NO:5 and sequences complementary thereto. It is preferred that said composition of matter comprises a buffer solution appropriate for the stabilization of said nucleic acid in an aqueous solution and enabling polymerase based reactions within said solution. Suitable buffers are known in the art and commercially available.

While the present invention has been described with specificity in accordance with certain of its preferred embodiments, the following EXAMPLES and TABLES serve only to illustrate the invention and are not intended to limit the invention within the principles and scope of the broadest interpretations and equivalent configurations thereof.

### TABLES 1-5

TABLE 1
Results of the Cox regression analysis for PITX2 according to
Example 1. Using stepwise regression the marker remains
in the model. P-values refer to the null-hypothesis
“hazard ratio equals zero”.

| | | | | Upper |
|---|---|---|---|---|
| | | Hazard | Lower Confidence | Confidence |
| Variable | P value | Ratio | Interval | Interval |
| PITX2 | 0.0043 | 2.222 | 1.284 | 3.845 |
| Disease stage | 0.0692 | 1.713 | 0.965 | 3.061 |
| Gleason category | 0.0107 | 1.798 | 1.146 | 2.821 |
| PSA | 0.075 | 1.254 | 0.977 | 1.609 |
| Nomogram | 0.0866 | 2.187 | 0.894 | 5.353 |
| category | | | | |

TABLE 2
Components for all QM assays according to Example 1

| Component | Company | Stock conc. | |
|---|---|---|---|
| Reaction buffer ROX | Eurogentec | 10x | |
| MgCl2 | Eurogentec | 50 | mM |
| DNTPs | MBI | 25 | mM each |
| Forward primer | TIB Molbiol | 6.25 | μM |
| Reverse primer | TIB Molbiol | 6.25 | μM |
| cg Probe | Eurogentec | 4 | μM |
| tg Probe | Eurogentec | 4 | μM |
| HotGoldStar-Taq | Eurogentec | 5 | U/μl |
| Water | Fluka | | |

TABLE 3A
Optimized Reaction conditions for all QM assays according to Example 1

| Gene | dNTPs | Buffer | MgCl₂ | Primers | Probes | Taq | Baseline | Threshold | Annealing |
|---|---|---|---|---|---|---|---|---|---|
| PITX2 | 250 μM | 1 x | 3 mM | 625 nM | 200 nM | 1U | 3/23 | 0.05 | 62° C. |

TABLE 3B
Cycle program for QM assays according to Example 1. For
annealing temperatures, see Table 3A.

| | T [° C.] | t | Cycles |
|---|---|---|---|
| Initial denat. | 95.0 | 10 min | |
| Denaturation | 95.0 | 15 sec | 45x (PITX2 |
| Annealing | Variable | 60 sec | 50x) |

TABLE 4
Clinical characteristics of the patient population according to
Example 1. Age is given as the mean, and all other variables
are given as the number of patients. Not all information was
available for all patients.

| Clinical Variable | | Baylor | Stanford | VMMC | Total |
|---|---|---|---|---|---|
| Age (mean) | | 61.1 | 61.7 | 61.1 | 61.3 |
| PSA | 0-4 | 25 | 33 | 18 | 76 |
| | 4-10 | 120 | 139 | 99 | 358 |
| | >10 | 60 | 72 | 30 | 162 |
| Gleason Score | 5-6 | 137 | 164 | 118 | 419 |
| | 7 | 37 | 44 | 19 | 100 |
| | 8-10 | 26 | 31 | 25 | 82 |
| Stage | Organ- | 110 | 211 | 113 | 434 |
| | confined | | | | |
| | Not organ- | 94 | 33 | 35 | 162 |
| | confined | | | | |

| | | | | |
|---|---|---|---|---|
| PSA-based recurrence | 22 | 10 | 13 | 45 |
| Decision to treat based | 3 | 14 | 4 | 21 |
| recurrence | | | | |
| Total Samples | 206 | 244 | 162 | 612 |

### EXAMPLE 1

The aim of the investigation was to confirm the significance of the gene PITX as a prognostic marker and to optimize methylation cut-offs. It was decided to investigate prostate cancer. The marker should be suitable to split patients who undergo prostatectomy into two groups: one with a high chance of PSA recurrence and one with a low chance of PSA recurrence. In addition, the markers should provide additional information to Gleason grade analysis. A marker meeting these criteria will have an important clinical role in selection of prostatectomy patients for adjuvant therapy. It was decided to undertake the analysis by means of methylation analysis on a real-time platform (QM Assay). The QM assay (=Quantitative Methylation Assay) is a Real-time PCR based method for quantitative DNA methylation detection. The assay principle is based on non-methylation specific amplification of the target region and a methylation specific detection by competitive hybridization of two different probes specific for the CG or the TG status, respectively. For the present study, TaqMan probes were used that were labeled with two different fluorescence dyes (“FAM” for CG specific probes, “VIC” for TG specific probes) and were further modified by a quencher molecule (“TAMRA” or “Minor Groove Binder/non-fluorescent quencher”). Evaluation of the QM assay raw data is possible by measuring absolute fluorescence intensities (FI) in the logarithmic phase of amplification.

The assay was used to analyze the methylation levels of 612 paraffin embedded prostatectomy samples from a cohort of node-negative patients from three institutions.

The primary aim of the invention was to provide a marker that can differentiate between patients with low chance for PSA recurrence after surgery and those with a high chance for PSA recurrence. The performance of these markers as compared to traditional prognostic indicators such as Gleason grading and stage information is also provided.

It is a further aim of the present invention to determine where the marker is most informative in relation to current clinical prognostic assessment and accordingly provide particularly preferred use embodiments of the present invention. It is particularly preferred that a molecular test according to the present invention is combined, either formally or informally, with information from other prognostic sources, and in the case of prostate cancer particular Gleason grading.

### Methods: QM Assay Description

The QM-assay was developed to enhance performance without drastically altering standard conditions in order to allow future multiplexing. Primer and probe concentrations, MgCl₂ concentration and annealing temperature were optimized under fixed buffer and polymerase conditions. The assay was designed and optimized to ensure quantitative methylation analysis of between 10 and 100 percent methylation. The assay products were checked on an agarose gel and no undesired products were detected. The results of the optimization procedure are shown in Tables 2 and 3.

### Oligonucleotide Sequences:

| | | | |
|---|---|---|---|
| Forward primer | gtaggggagggaagtagatgtt | | |
| Reverse primer | ttctaatcctcctttccacaataa | | |
| CG-probe | agtcggagtcgggagagcga | Label 5- FAM | Label 3′-TAMRA |
| TG-probe | agttggagttgggagagtgaaaggaga | Label 5- VIC | Label 3′-TAMRA |

### Sample Set

Paraffin-embedded prostatectomy tissue samples from 612 patients were analyzed, see Table 4. The samples were provided by the Baylor College of Medicine SPORE, Stanford University Department of Urology, and Virginia Mason Hospital in Seattle. The samples from Stanford and Virginia Mason were prepared by first finding the surgical block with the highest percent tumor, then sectioning the block. Three tubes were prepared, each with three 10 micron thick sections. The procedure was slightly different at Baylor. A core of tissue was removed from the tumor within the prostatectomy block, and then this core was cut into 10 micron sections. Ten sections were included into each of three tubes.

An adjacent section was mounted on a slide and H&E stained for histological analysis. A pathologist reviewed these slides for an independent determination of Gleason grading and percent tumor. The Gleason results were used for all analyses in this report. The original provider Gleason values are available, but they were not used for analysis due to known and hypothetical biases among the providers. Stanford, for instance, uses a percentage Gleason 4/5 for reporting grade, while the other two providers use the traditional system. The measured Gleason values provided an independent and uniform measurement.

A few samples were found to have no tumor cells on the H&E slide, and these patients were omitted from the analysis. In addition, we found a few patients that did not have a PSA nadir after surgery. These patients were also excluded from the study. In total, 612 patients were included in the data analysis.

Due to their coring technique, the percent tumor of the samples provided by Baylor were higher than the other providers.

All patients, aged 40-80, undergoing surgery at the three institutions during certain years were included in the study, with the exception of patients who received neo-adjuvant or adjuvant therapy (before PSA rise) and patients with positive nodes at the time of surgery. For Baylor, the time period was 1993-1998, for Virginia Mason it was 1996-2000, and for Stanford it was 1996-1999.

The overall cohort is similar to other prostatectomy cohorts described in the literature, such as the cohort collected by William Catalona and described in 2004 (Roehl et al.). The patient cohorts from each provider are similar for nearly all clinical parameters. One exception is the type of recurrence. While other institutions typically wait until the patient's PSA rises to 0.2 ng/ml or higher after surgery, the Stanford Department of Urology treats many patients when their PSA rises to 0.05. Therefore, Stanford has a higher rate of recurrence based on the decision to treat criteria and a lower rate of recurrence based on the PSA level (0.2 ng/ml) criteria. See section 6.1 for a summary of the event definition criteria.

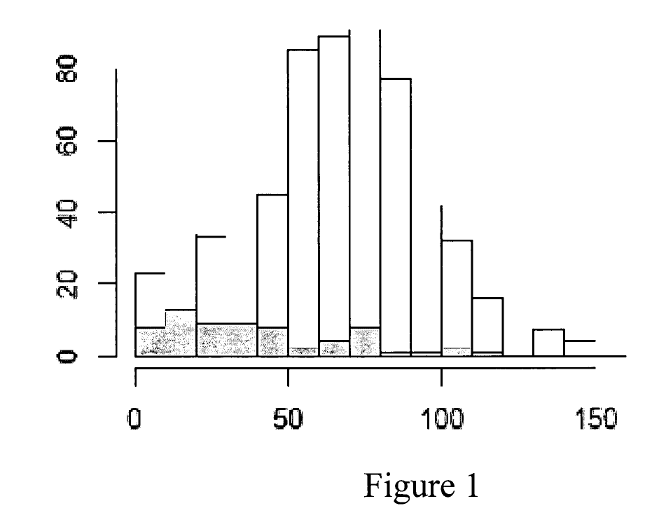

FIG. 1 provides a histogram of follow-up times for the patient cohort (all three providers included). The white bars consist of the patients who did not have a recurrence before they were censored, and the shaded bars consist of the patients who experienced recurrence. By selecting patients who received surgery from 1993-2000, we have ensured that the median follow-up time of the cohort (66 months) is long enough to have a significant number of patients who have relapsed.

For deparaffination, the 627 provided PET samples were processed directly in the tube in which they were delivered by the providers. One ml (Virginia Mason and Baylor) or 1.8 ml (Stanford) of limonene was added to each tube and incubated at room temperature for 10 minutes in a thermomixer with occasional vortexing. The samples were centrifuged at 16,000×g for 5 minutes. The limonene supernatant was removed, and if no pellet was detected, centrifugation was repeated at higher speed and the remaining limonene was removed. For samples from Stanford, the deparaffination process was repeated once with 1.6 ml of limonene to get rid of residual paraffin.

For lysis of the tissue, 190 μl lysis buffer and 20 μl proteinase K was added to each deparaffinated sample. For Stanford samples, 570 μl lysis buffer and 60 μl proteinase K was used. After vortexing, samples were centrifuged briefly and incubated on a thermoshaker at 60° C. for 40 hours. After the incubation, samples were checked to ensure that lysis was complete, and the proteinase was then inactivated at 95° C. for 10 minutes. If the lysed samples were not directly used for DNA extraction, they were stored at −20° C.

The lysates were randomized based on the sample provider and PSA recurrence. The DNA was isolated using a QIAGEN DNeasy Tissue kit with a few modifications. 400 μl buffer AL/E was distributed to collection tubes and 200 μl of lysate were added. The samples were mixed by shaking for 15 seconds. The lysate/buffer mixtures were applied to the 96-well DNeasy plate columns. The plate was sealed and centrifuged at 5790×g for 10 minutes. The columns were washed once with 500 μl of AW1 and then 500 μl AW2. The DNA was eluted with 120 μl buffer AE. Therefore, the final volume of extracted DNA was approximately 120 μl. The DNA was stored at −20° C.

### Bisulfite Treatment

The CFF real-time PCR assay was used to quantify the DNA concentration of the samples after extraction.

### CFF Sequence:

TAAGAGTAATAATGGATGGATGATGGATAGATGAATGGATGAAGAAAGAA
AGGATGAGTGAGAGAAAGGAAGGGAGATGGGAGG (84 bp)

| | |
|---|---|
| CFF-Forward primer | TAAGAGTAATAATGGATGGATGATG |
| CFF-Reverse primer | CCTCCCATCTCCCTTCC |
| CFF TaqMan probe | ATGGATGAAGAAAGAAAGGATGAGT |

The inventors adjusted the concentration of each genomic DNA sample so that 1 ug of CFF1 measured DNA was present in 44 μl. The bisulfite treatment of genomic DNA derived from paraffin embedded tissue was performed using a 96 well protocol. Forty-four μl genomic DNA (with approximately 1 μg of amplifiable DNA), 83 μl 4.9M bisulfite solution (pH 5.45-5.5), and 13 μL DME solution were pipetted into the wells of the plate. The samples were thoroughly mixed then placed in a thermocycler with the following program:
5:00 min denaturation of DNA at 99° C.
22:00 min incubation at 60° C.
3:00 min denaturation of DNA at 99° C.
1:27:00 hours incubation at 60° C.
3:00 min denaturation of DNA at 99° C.
2:57:00 hours incubation at 60° C.
Cooling at 20° C.

After the incubations, each sample was divided into two 70 μL aliquots. Each aliquot was combined with 280 μL of prepared Buffer AVL/Carrier RNA and 280 μL ethanol. The wells were sealed and the samples were mixed vigorously for 15 seconds. The plate was incubated for 10 minutes at room temperature. The first aliquot was applied to the QIAamp 96 plate and the plate was centrifuged for four minutes at 5790×g. The process was repeated with the second aliquot so that both aliquots were applied to the same binding column. The columns were washed with 500 μL buffer AW1, then 500 μL 0.2 M NaOH, and then twice with 500 μL buffer AW2. The DNA was eluted with 100 μL elution buffer (Qiagen) pre-heated to 70 deg C. The bisDNAs were stored at −20° C.

The bisulfite treated DNA samples were stored in 8×96 well plates (plate 01-08). The samples and controls were combined onto two 384-well PCR reaction plates for each QM assay. Each QM assay plate contained the samples of 4×96 well plates (85 wells actually used per plate) and 1×96 well plate with standard DNA (7 mixtures of the calibration DNA and water for the no template control PCR reaction). The QM assay plates were run three times.

The 384-well PCR plates were pipetted with the TECAN workstation. The pipetting program transferred first 10 μl of the mastermix and then 10 μl of the respective DNA into the designated well. The master mix was pipetted in a falcon tube and distributed to 8×500 μl screw cap vials for automatic pipetting with TECAN workstation.

All QM assays were run on an ABI TAQMAN 7900HT real-time device (SDS 2.2. software) with a reaction volume of 20 μl. PITX2 and CCND2 assays were run with 9600 emulation, and the other assays were not. An automatic sample setup was used to transfer the correct sample names and detector/reporter dyes to the TAQMAN software. The cycling conditions were manually adjusted and ROX was used as passive reference dye. All 384 well PCR plates we analyzed with the SDS2.2 software using the manual analysis settings (baseline setting with start and stop values and manual threshold) to produce results files for each run individually.

### Methods: Evaluation of Marker Performance

### Definition of Events

After a successful prostatectomy on a patient with non-metastatic disease, there should be no prostate cells left in his body and therefore his PSA levels should drop to zero. A patient's PSA levels are typically measured every 6-12 months after surgery to ensure that the patient remains free of prostate cancer. If PSA becomes detectable and rises to a certain level, the doctor and patient may decide on additional therapy. Therefore, the return and rise of PSA levels are the primary indication of disease recurrence.

A post-surgical PSA relapse is typically indicated by either a gradual or rapid rise in levels over a series of sequential tests. Depending on the clinical characteristics of the patient or the approach of the institution, patients may be treated as soon as PSA is detected, when it reaches a certain threshold, or when clinical symptoms accompany the PSA rise. Most institutions consider a PSA level of 0.2 ng/ml to be significant, and if a patient's PSA reaches this level and is confirmed to be rising in subsequent tests, he will be offered additional therapy. Stanford Department of Urology, one of the sample providers, considers 0.05 ng/ml to be a PSA recurrence, and considers treatment for patients when their PSA reaches this level.

An event in this study includes all PSA-based recurrences. A PSA level of 0.2 ng/ml, confirmed in subsequent tests, has been demonstrated to provide the best sensitivity and specificity for detection of recurrence (Freedland et al. 2003). Rise of PSA to this level normally precedes any development of clinical recurrence; therefore, nearly all of the patients in this study are free of clinical recurrence at the time of PSA recurrence. Because Stanford often treats patients with PSA recurrence before they reach this cut-off of 0.2 ng/ml, many of their recurrence patients would be censored in the present study if the PSA level of 0.2 ng/ml was the only considered event. Therefore, patients from any of the three institutions who receive therapy due to PSA levels are also considered an event in this study.

To summarize, an event is defined in the present study as any rise in PSA to 0.2 ng/ml (confirmed in subsequent test) OR a decision to treat the patient based on PSA criteria.

### Raw QM Data Processing

All analyses in this report are based on the CT evaluation. Assuming optimal real-time PCR conditions in the exponential amplification phase, the concentration of methylated DNA (Cₘₑₜₕ) can be determined by

$${C}_{meth} = \frac{100}{1 + {2}^{( {CT}_{CG} - {CT}_{TG} )}} [ \% ] ,$$

where
CT_{CG} denotes the threshold cycle of the CG reporter (FAM channel) and
CT_{TG} denotes the threshold cycle of the TG reporter (VIC channel).

The thresholds for the cycles were determined by visual inspection of the amplification plots (ABI PRISM 7900 HT Sequence Detection System User Guide). The values for the cycles (CT_{CG} and CT_{TG}) were calculated with these thresholds by the ABI 7900 software. Whenever the amplification curve did not exceed the threshold, the value of the cycle was set to the maximum cycle e.g. 50.

The R software package, version 2.2. (Gentleman and Ihaka 1997), was used for the statistical analysis. In addition, we used the “survival” package, version 2.11-5 (http://cran.at.r-project.org/src/contrib/Descriptions/survival.html), for survival analysis.

Proprietary code was used for k-fold-cross validation, ROC analysis and plot functions.

Each dataset is represented in a proprietary data object, called “Annotated Data Matrix” (ADM). This data object contains the measurements after quality control and averaging, as well as all necessary annotations for the samples and assays.

### QM Assay Calibration Curves

A series of mixtures of methylated MDA-DNA and unmethylated MDA-DNA, ranging from 0 to 100 percent methylated, were included in triplicate on each QM PCR plate. These DNAs were used to ensure uniform QM assay performance on all PCR plates. All assays showed strong quantitative abilities between 10 and 100%, and some assays were able to consistently distinguish 5% methylated DNA from unmethylated DNA.

### Statistical Methods

After quality control, each assay was statistically analyzed.

### Cox Regression

The relation between recurrence-free survival times (RFS) and covariates were analyzed using Cox Proportional Hazard models (Cox and Oates 1984; Harrel 2001).

The hazard, i.e. the instantaneous risk of a relapse, is modeled as

h(t|x)=h₀(t)·exp(βx) (3)

and

h(t|x₁, . . . , xₖ)=h₀(t)·exp(β₁x₁+ . . . +βₖxₖ) (4)

for univariate and multiple regression analyses, respectively, where t is the time measured in months after surgery, h₀(t) is the (unspecified) baseline hazard, xᵢ are the covariates (e.g. measurements of the assays) and βᵢ are the regression coefficients (parameters of the model). βᵢ will be estimated by maximizing the partial likelihood of the Cox Proportional Hazard model

Likelihood ratio tests are performed to test whether methylation is related to the hazard. The difference between 2Log(Likelihood) of the full model and the null-model is approximately □²-distributed with k degrees of freedom under the null hypotheses □₁= . . . =□ₖ=0.

The assumption of proportional hazards were evaluated by scaled Schoenfeld residuals (Thernau and Grambsch 2000). For the calculation, analysis and diagnostics of the Cox Proportional Hazard Model the R functions “coxph” and “coxph.zph” of the “survival” package are used.

### Stepwise Regression Analysis

For multiple Cox regression models a stepwise procedure (Venables and Ripley 1999; Harrel 2001) was used in order to find sub-models including only relevant variables. Two effects are usually achieved by these procedures:
Variables (methylation rates) that are basically unrelated to the dependent variable (DFS/MFS) are excluded as they do not add relevant information to the model.
Out of a set of highly correlated variables, only the one with the best relation to the dependent variable is retained.
   Inclusion of both types of variables can lead to numerical instabilities and a loss of power. Moreover, the predictor's performance can be low due to over-fitting.

The applied algorithm aims at minimizing the Akaike information criterion (AIC), which is defined as

AIC=2□maximized log-likelihood+2□#parameters.

The AIC is related to the performance of a model, smaller values promise better performance. Whereas the inclusion of additional variables always improves the model fit and thus increases the likelihood, the second term penalizes the estimation of additional parameters. The best model will present a compromise model with good fit and usually a small or moderate number of variables. Stepwise regression calculation with AIC are done with the R function “step”.

### Kaplan-Meier Survival Curves and Log-Rank Tests

Survival curves were estimated from RFS data using Kaplan-Meier estimator for survival (Kaplan and Meier, 1958). Log-rank tests (Cox and Oates 1984) are used to test for differences of two survival curves, e.g. survival in hyper- vs. hypomethylated groups. In addition, a variant of the Log-rank test usually referred to as the Generalized Wilcoxon test was applied (for description see Hosmer and Lemeshow 1999). For the Kaplan-Meier analysis the functions “survfit” and “survdiff” of the “survival” package are used.

Independence of Single Markers and Marker Panels from Other Covariates

To check whether the present markers give additional and independent information, other relevant clinical factors were included in the Cox Proportional Hazard model and the p-values for the weights for every factor were calculated (Wald-Test) (Thernau et al. 2000). For the analysis of additional factors in the Cox Proportional Hazard model, the R function “coxph” is used.

### Density Estimation

For numerical variables, kernel density estimation was performed with a Gaussian kernel and variable bandwidth. The bandwidth is determined using Silverman's “rule-of-thumb” (Silverman 1986). For the calculation of the densities the R function “density” is used.

### Analysis of Sensitivity and Specificity

The method of calculating sensitivity and specificity using the Bayes-formula was based on the Kaplan-Meier estimates (Heagerty et al. 2000) for the survival probabilities in the marker positive and marker negative groups for a given time T_{Threshold}. The ROCs were calculated for different reference times T_{Threshold} (3 year, 4 years, 5 years, 6 years).

k-fold Crossvalidation

For the analysis of model selection and model robustness k-fold crossvalidation (Hastie et al. 2001) was used. The set of observations is randomly split into k chunks. In turn, every chunk was used as a test set, whereas the remaining k−1 chunks constitute the training set. This procedure is repeated m times.

### Results

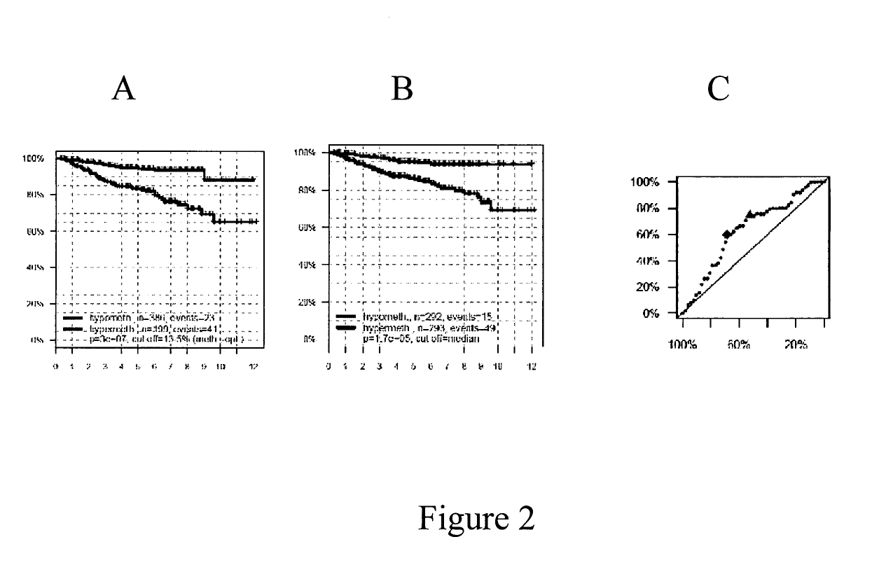

The 605 samples were processed as described above. All samples were analyzed by the QM assays with three replicates. The data was filtered for quality control, and analyzed as described in the methods section. The clinical performance of each marker is summarized below and the Kaplan-Meier survival curves and ROC curves according to FIG. 2. P-values for comparison of survival curves reported in the graphs are based on the ordinary Log-rank test. The results of using the Generalized Wilcoxon test are essentially the same (data not shown).

The performance of the markers was first examined using the median methylation level as a cut-off. Since this cut-off was fixed before looking at the data, the p values can be used to judge the performance of the markers. Any marker with a significant p value using the median methylation as a cut-off is considered to be validated. The median methylation level might not be the best cut-off for all markers, and for these markers the prognostic separation can be further optimized by choosing the methylation cut-off that results in the lowest p value. Since the cut-off is optimized specifically for p value, the p value no longer can be used to indicate statistical significance.

For judging the significance of the marker performance using the median methylation as a cut-off, we used a p value of 0.005 (assuming correction for 10 markers and panels). Based on p-value and event separation, PITX2 is a strong candidate.

FIG. 2 A shows the Kaplan-Meier survival analysis of the PITX2 marker of the 585 patient samples that passed the quality control filter using the optimized methylation cut-off value (13.5%). FIG. 2B shows the Kaplan-Meier survival analysis of the PITX2 marker using the predefined median methylation value as a cut-off, the p-value was 0.000017. FIG. 2C shows the ROC curve analysis of the PITX2 marker after 5 years of follow-up. The median methylation cut-off is marked as a triangle, and the optimized methylation cut-off is shown as a diamond. The AUC was 0.64.

Several clinical prognostic factors are commonly used for assessing prostate cancer. Histological analysis of the tumor with quantification of the tumor differentiation state using the Gleason grading system is a particularly important prognostic indicator in current clinical practice. The analysis was continued by determining whether the markers could improve Gleason analysis by subdividing patients within a Gleason category. We also investigated whether the markers could add information to other prognostic indicators, such as nomogram risk estimation (Han et al. 2003) and disease stage.

For these analyses, we used Kaplan-Meier analysis to determine whether the PITX2 is still informative on population sub-groups, and Cox regression analysis to determine whether the markers provide information independent of the prognostic clinical variables. Gleason score was divided into three groups (6 or lower, 7, and 8 through 10), stage was divided into two groups (T2/organ-confined and T3/non-organ confined), PSA was divided into four groups (0 to 4 ng/ml, 4 to 10 ng/ml, 10 to 20 ng/ml, and greater than 20 ng/ml), and nomogram estimation of 5 year PSA-free survival was divided into two groups (90 to 100% and 0 to 89%).

With Cox regression modeling, PITX2 is a valuable prognostic marker independent of other clinical prognostic information (Table 1). In other words, PITX2 methylation adds more information to Gleason than either PSA or disease stage. The hazard ratio for PITX2 is 2.2. In the survival analysis of sub-groups, PITX2 has the potential to be a significant marker for all prostate cancer patients.

It is particularly interesting to see strong separation within the patient sub-group with organ-confined disease (FIG. 96). Patients with organ-confined disease (T2) should be cured by surgery. Those that are not cured by surgery must have had some cells leave the prostate before surgery, and therefore had tumor cells with aggressive characteristics early in the development. PITX2 can separate the T2 group into a hypomethylated group with a very small chance for recurrence (˜5%) and a hyper-methylated group with a prognosis more like T3 patients.

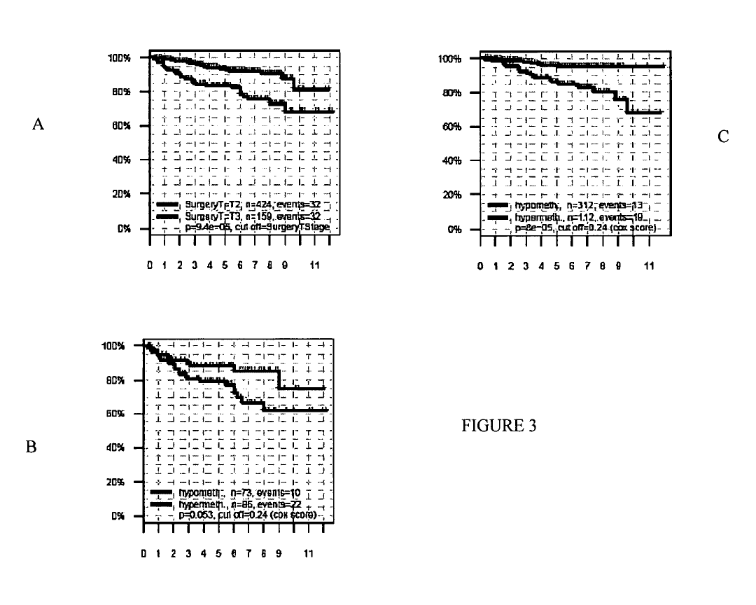

FIG. 3 shows the survival analysis of PITX2 performance on sub-populations based on stage. The upper left plot shows the performance of disease stage as a prognostic marker. The upper right plot shows the performance of PITX2 on pT2 patients. The lower left plot shows the performance of PITX2 on pT3 patients.

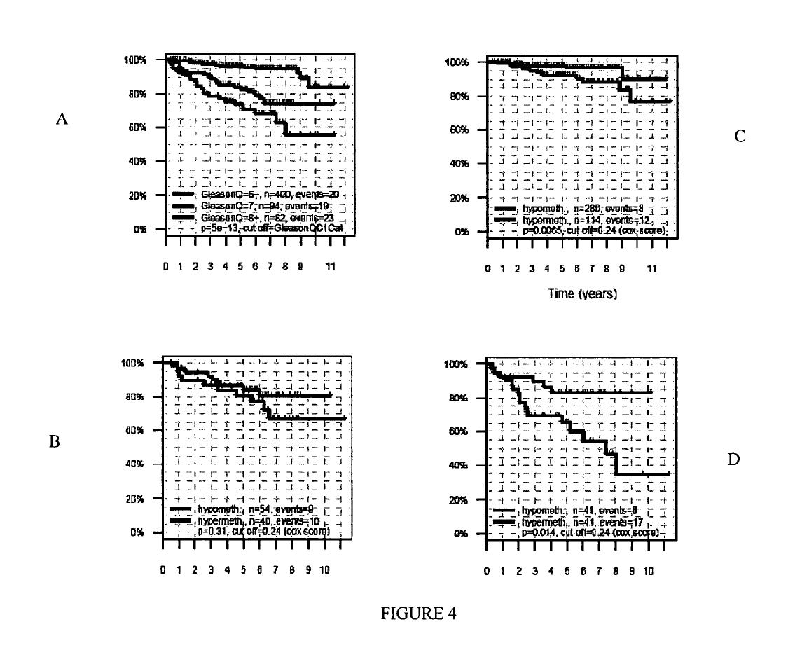

PITX2 is also capable of stratifying patients within Gleason sub-categories. FIG. 4 shows that survival analysis on low Gleason patients (Score 5 or 6) and high Gleason patients (Score 8, 9, or 10) results in low p values. Patients with high Gleason scores are currently candidates for clinical trials on post-surgical adjuvant therapies. But the PITX2 values suggest that this is not a uniform group. PITX2 hypomethylated, high Gleason patients have 85% probability of disease free survival at ten years, while hypermethylated high Gleason patients have a very low chance (˜35%). These patients with high likelihood for disease recurrence are the patients who should be selected for adjuvant therapy or clinical trials.

FIG. 4 shows the survival analysis of PITX2 performance on sub-populations based on Gleason score categories. The upper left plot shows the performance of Gleason score as a prognostic marker. Gleason 5 and 6 patients are in light grey, Gleason 7 patients are in dark-grey, and Gleason 8, 9, and 10 patients are in black. The upper right plot shows the performance of PITX2 on Gleason 5 and 6 patients. The lower left plot shows the performance of PITX2 on Gleason 7 patients. The lower right plot shows the performance of PITX2 on Gleason 8, 9, and 10 patients.

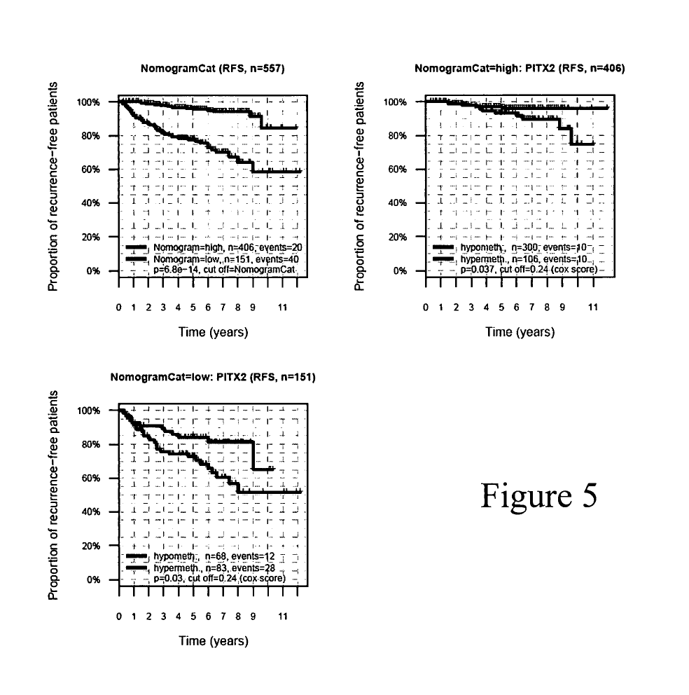

Prostate cancer nomograms are created based on large cohorts of patients. They mathematically combine information from stage, Gleason, and pre-operative PSA levels into one prognostic indicator. As FIG. 5 shows, the nomogram by itself is very strong. But PITX2 is capable of further sub-dividing the patients.

FIG. 5 shows the survival analysis of PITX2 performance on sub-populations based on nomogram risk estimation. The upper left plot shows the performance of the nomogram as a prognostic marker. The upper right plot shows the performance of PITX2 on patients with a 90% chance of 5-year PSA-free survival according to the nomogram. The lower left plot shows the performance of PITX2 on patients with less than 90% chance of 5-year PSA-free survival according to the nomogram.

PITX2 shows significant prognostic information when the median methylation level is used as a cut-off. Setting the methylation cut-off even higher than the median improves the performance. This has the effect of decreasing the marker positive group and increasing the specificity of the test.

The patients whose samples were analyzed in this study are representative of the population who would be targeted for a prostatectomy test. Therefore, it is possible to speculate on the information these markers could provide for future patients. PITX2, for example, has a sensitivity of around 60% and a specificity of 70%. In the Kaplan-Meier analysis in FIG. 2, the marker positive group has approximately three times the risk of recurrence after ten years that the marker negative group has. In FIG. 4, Gleason 8-10 patients that are positive for PITX2 have a 65% chance for PSA recurrence in 10 years. In contrast, the Gleason 8-10 patients who were marker negative had only a 15% chance of PSA relapse. The addition of the methylation marker information to the Gleason stratification will allow clinicians to identify a poor prognosis sub-group who can most benefit from adjuvant therapy. If the marker is incorporated into the patient selection procedure for adjuvant therapy clinical trials, clinicians may begin to see a clear benefit to the addition of early adjuvant treatments for poor prognosis patients.

In addition to adding information to Gleason, PITX2 can also stratify patients with organ-confined disease. Patients with disease that is truly confined to the organ will be cured by complete removal of the organ. Patients with disease that appears to be confined to the organ, but have undetected micrometastases, will not be cured by surgery. These two groups of patients, both with small operable lesions, have tumors with very different capacities for metastases. PITX2 seems to be detecting these underlying differences in basic tumor aggressiveness. The ability of PITX2 to add information to currently used markers is essential. Gleason and staging already provide significant prognostic information, a new test that would not replace but complement these traditional sources of information is both more valuable and more likely to be readily adopted in clinical practice.

In the analysis on sub-groups of patients, the marker often seemed strongest on patients with poor prognosis based on traditional clinical variables. Gleason 8-10 patients and patients with low nomogram probability for PSA free survival are well stratified by the present marker into good and poor prognosis groups. For a prostatectomy test, these are the ideal patients to target, since the test would be used to select a group of poor prognosis patients who can most benefit from adjuvant therapy. Overall, this analysis demonstrates that the PITX2 marker is especially well suited for identifying poor prognosis patients.

1. A method for providing a prognosis of a subject with a cancer comprising:
obtaining a biological sample from a subject having a cancer other than breast cancer,
determining using a suitable assay, an expression status of the gene PITX2 in said sample, and
determining from the expression status a prognosis of said subject, wherein over-expression or hypermethylation is indicative of negative prognosis.

2. A method according to claim 1, wherein said cancer is at least one selected from the group consisting of bladder cancer, colorectal cancer, endometrial cancer, kidney or renal cell cancer, leukemia, lung and bronchial cancer, melanoma, non-Hodgkin's lymphoma, pancreatic cancer, prostate cancer, skin cancer and thyroid cancer.

3. The method of claim 1, wherein at least one further prognostic variable is considered in determining the prognosis.

4. The method of claim 1, wherein said prognosis is determined with respect to least one factor selected from the group consisting of overall patient survival, disease- or relapse-free survival, tumor-related complications and rate of progression of tumor.

5. The method of claim 1, further comprising based on the prognosis
determining a suitable treatment for said subject.

6. The method of claim 1, wherein the sample is at least one selected from the group consisting of cells or cell lines, histological slides, biopsies, paraffin-embedded tissue, bodily fluids, ejaculate, urine, blood, sputum, stool, tissue, colon tissue, prostate tissue, lung tissue, and liver tissue.

7. The method of claim 1, wherein the PITX2 expression status is determined by measuring the level of at least one of PITX2 mRNA, cDNA or polypeptide.

8. The method of claim 7 wherein the expression is determined by use of at least one technique selected from the group consisting of Northern blot analysis, reverse transcriptase PCR, real-time PCR, RNAse protection, and microarray analysis.

9. The method of claim 1, wherein said PITX2 expression status is determined by determining the level of methylation or methylation status of one or more CpG positions within at least one of said gene and a regulatory region thereof.

10. The method of claim 9 comprising contacting genomic DNA isolated from the biological sample with at least one reagent, or series of reagents that distinguishes between methylated and non-methylated CpG dinucleotides within at least one target region of the genomic DNA, wherein the target region comprises, or hybridizes under stringent conditions to a sequence of at least 16 contiguous nucleotides of the PITX2 gene and/or regulatory regions thereof, wherein said contiguous nucleotides comprise at least one CpG dinucleotide sequence, and wherein determining the level of methylation or methylation status is afforded.

11. The method of claim 10 comprising:
isolating genomic DNA from the biological sample;
treating the genomic DNA, or a fragment thereof, with one or more reagents suitable to convert 5-position unmethylated cytosine bases to uracil or to another base that is detectably dissimilar to cytosine in terms of hybridization properties;
contacting the treated genomic DNA, or the treated fragment thereof, with an amplification enzyme and at least two primers comprising, in each case a contiguous sequence at least 18 nucleotides in length that is complementary to, or hybridizes under moderately stringent or stringent conditions to a sequence selected from the group consisting of SEQ ID NOS:2-5, contiguous portions thereof and complements thereof, wherein the treated DNA or a fragment thereof is either amplified to produce one or more amplificates, or is not amplified;
determining, based on the presence or absence of, or on a quantity or property of said amplificate, the methylation state of at least one CpG dinucleotide sequence of the gene PITX2 or an average, or a value reflecting an average methylation state of a plurality of CpG dinucleotide sequences of the PITX2 gene; and
d) determining from said methylation state the prognosis of said subject.

12. A treated nucleic acid derived from SEQ ID NO: 1, wherein the treatment is suitable to convert at least one unmethylated cytosine base of the genomic DNA sequence to uracil or another base that is detectably dissimilar to cytosine in terms of hybridization.

13. A nucleic acid, comprising at least 16 contiguous nucleotides of a treated genomic DNA sequence selected from the group consisting of SEQ ID NOS:2-5 contiguous portions thereof and complements thereof, wherein said nucleic acid is not identical or complementary to SEQ ID NO: 1, wherein the treatment is suitable to convert at least one unmethylated cytosine base of the genomic DNA sequence to uracil or another base that is detectably dissimilar to cytosine in terms of hybridization.

14. The nucleic acid of any one of claims 12 and 13, wherein the contiguous base sequence comprises at least one CpG, TpG or CpA dinucleotide sequence.

15. The nucleic acid of any one of claims 12 and 13, wherein the treatment comprises use of a reagent selected from the group consisting of bisulfite, hydrogen sulfite, disulfite, and combinations thereof.

16. An oligomer, comprising a sequence of at least 9 contiguous nucleotides that is complementary to, or hybridizes under moderately stringent or stringent conditions to a treated genomic DNA sequence selected from the group consisting of SEQ ID NOS:2-5 contiguous portions thereof and complements thereof, wherein said nucleic acid is not identical or complementary to SEQ ID NO:1.

17. The oligomer of claim 16, comprising at least one CpG, CpA or TpG dinucleotide.

18. A kit for use in for use in providing a prognosis of a subject with a cancer other than breast cancer, comprising a means for detecting the polypeptides of the PITX2 gene.

19. The kit according to claim 18, comprising: (a) a means for detecting the polypeptides of the PITX2 gene; (b) a container suitable for containing the said means and a biological sample of the patient comprising said polypeptides wherein the means can form complexes with the polypeptides; (c) a means to detect the complexes of (b); and optionally (d) instructions for use and interpretation of the kit results.

20. A kit for use in for use in providing a prognosis of a subject with a cell proliferative disorder, comprising: a means for measuring the level of mRNA transcription of the PITX2 gene.

21. The kit according to claim 20, comprising: (a) a means for measuring the level of mRNA transcription of the PITX2 gene; (b) a container suitable for containing the said means and a biological sample of the patient comprising mRNA of the PITX2 gene wherein the means are able to hybridize to the transcription products of said gene; (c) a means for detecting the complexes of (b); and optionally (d) instructions for use and interpretation of the kit results.

22. A kit comprising
at least one bisulfite reagent; and
at least two nucleic acid molecules comprising, in each case a contiguous sequence at least 16 nucleotides that is complementary to, or hybridizes under moderately stringent or stringent conditions to a sequence selected from the group consisting of SEQ ID NOS:2-5 contiguous portions thereof.

23. A composition comprising:
a nucleic acid comprising a contiguous sequence at least 18 bases in length of a chemically pretreated genomic DNA according to a sequence selected from the group consisting of SEQ ID NOS:2-5 contiguous portions thereof and complements thereof; and
a buffer comprising at least one of magnesium chloride, dNTP, Taq polymerase, and an oligomer, oligonucleotide or peptide nucleic acid (PNA)-oligomer, said oligomer, oligonucleotide or (PNA)-oligomer comprising in each case at least one contiguous base sequence having a length of at least 9 nucleotides which is complementary to, or hybridizes under moderately stringent or stringent conditions to a pre-treated genomic DNA according to a sequence selected from the group consisting of SEQ ID NOS:2-5 contiguous portions thereof and complements thereof.

24. (canceled)


SEQUENCE LISTING (5 sequences)

SEQ ID NO: 1 - DNA, 28536 bp, Homo Sapiens
gctctgggagctaggtttctctcgctggatgggaaaaggaggtgcggaaaggggcaggcggcctgcgggctagcggggatgagagttgtggggagaacggaggagagagtaaaattattccttttggaagtccaagggaagggagctgaggtgagagaaatggcctctcttaggtcccaagtctccgcccccagcctgcaccactcaagtcggggaagtgacaactcttcctaaacctcgacttccattttttgtttcctgcagattgattgatgtcctcgcgttgttctagttcagcaaccaaactaggccgaggtacacctcttttacccagagctcccaaatcactcaccaacctgcaggagacctaaatcctccgggcaggtctcgttagcgcttccagcccccatccccaaatcccctcttctagctccttccttcccctccgccctttggccctccccgccccctaccaggctgagaggcggagaggccgggaccgctgggccctgcggctccagaagccaaagtccgcccgtggggaccgttttcctctccactgctctcaagtgaacaacatggtgggaggtgaccctcgtccaaagggcttttatctgccccccactgctgcgtgatccctccgcaagtcggaggcagagctttaaaaagaaagaaagtgtgcgttgagcactgatcttaaatcggggacattatctgcgatctctttaagtggcagcagcagtttcttgcagaacaaagactcagtgttcgagctccaaaagcttctaggtaaagtttcattcagatgaaagcaactaaggcgaaaaaacaatgatattgtcggctccagaaaatcggcggttactttttgctctaagttccccagcgtggtgtttgttctcgcccactttggttgtgcggggggttgacaagcataacaaaagatctcagcacatccctccccccacctccacaacgaccctcctagcgccatgaggatgaattctctgggactaagtggtatttgtttctatttgttatgaaatcaaatttcacatttttttaaagctgaaaatcgcggataaagaaggatcgggtgcttaaagttcatttaaacactcacaccgaactcattccctgtttagatgtcagaggatggcagggagggccagggctgtaattcatcttgatttgcttcattgtcctgcagtttgcaaactataatcctgtataatttcaggaacaagcaggtttagaattaatgggtcaatattatttaaaacaaaacaccgggagcatgacctttgcctcaactacatattgctcgacaagactcaggaaactccactctaacctctcaacccctgagttctttgagaaactgaagggctgtagttattagaaatcatctttgtttcttttaatgtctccaaaggatttggaaatagtaatgcaatataacatgaaggatctctctacttaagcctgaagataaacgtggaagcctaaatattttgaactggagatgtttccctgtaaatttattatagatgtattcctcctgagagaaatgcatataaactatacatgtatacatgcacaaatatttctccagaacaatttgctagaggtgtaggtttccccgtaaaggagtaaacttgagagtcattttaagctgatggacttgctaaatttctttcttcttttttctttttcatattatttgctagccataatggaatcctctaggtttaagccaaagaaaaattggagagacaaaattagattttgtagcccttttcccccccgggaatgcctttttttttctttttagtttctgatgaatggctatcatttatttctaccaaatttaaataaggactgctgccttgtatgtttaactaggcaggcagagggaactggtttgtttaggaagcagtgactgagatgtcctggccaagttagtgacagaggaggggagaaagaatccagaccaatttgtatgcagtatattttactcccatgaaataaaacacatttgtttcatatttgctgaaaagtaaaacaataatattgtacgaaatgttatacacagggtaggttgtacatagcagtttcagaaacatcattgcatccaccagagaaactattctaaaactgatattcacacattttttataataataataatatgttagaaacatacagtgtggcatttagtatatacactcccttgctcgcaagcgaaaaatcctaatcgcttctgtataacatgctttattttaaagcctaacctttaaaaacactgttgtgatattactaacaactgcttttataaaattaatttgacatttcgatatatatacatcctttcagtcatttaaatgttaacaatgctaaacttaaaaaataacaagcttatagtaatgttaaaatgtcatatccagtcaaacatttgtttgtgtatgtgtccttgcaactgttagaaatacttgtagtgaaagatgtcagacactgaggacatccctttgaaatcaaaggagctctctctttgattcagtggtttccttttctctatatagcttctctttctctccctttctttagtgcccacgaccttctagcataattcccagtctttcaagggcggagttgccccatccggcaaggtcctaggatcccggcgctgtgggtgcggctcacacgggccggtccactgcatactggcaagcactcaggttggaggccgggttctgcacgctggcgtagccgaagctggagtgctgctttgctttcagtctcaggctggccaggctcgagttacacgtgtccctataaacatacggaggagtcggcggcgcgtaaggacaggcaggcgtcggcaccgcggaattcagcgacgggctactcaggttgttcaagttattcaggctgttgagactggagcccgggacgcctgtcactgctgagggcaccatgctggacgacatgctcatggacgagatagagttgggtggggaaaacatgctctgtgatgacagggggttgacgttcatagagttgaagaaggggaagctcttggtggatagggaggcggatgtaaggcccttggcggcccagttgttgtaggaatagcctgggtacatgtcgtcgtagggctgcatgagcccattgaactgcggcccgaagccattcttgcatagctcggcctgctggttgcgctccctctttctccatttggcccgacgattcttgaaccaaacctgggggcggttggggcaagggagcaaacagatgccacagtgcagattactaaaacttccatcggaggccaacccccgccttcccccgacacacacgctagcgcactcacacaccctggcctcgcttcactgcaccgccctgcacaccaagataccagggccagctttcagttactggcccgggtctccaccaagcgcaggagacctggtctgctctggcctgcgagctgggactcggagctacgccacaaacctcagccgaacgcatggagacctgcggacggtttgatcactcagccaggcgtttctccaggtccaaaaacacttaatgtaaaacaaacgcggggcagcaggcttttccaacccttcccggggcaccttgcaaacttgcttccattccaaagccacagacccacggatgaggagaaggggctggaagggcactagaggatcgctctttctcccacgcaattcctcccttccttccctgacctccactgtcgtcccccaccccctggtacgtgctcccttaacagggactaggccgccaacactctttctcgcctagcaaaacaaccaaataaagagcaaaagaccacctcttcgtcagctcgttaactccaggagcttggcatattaaactccgggaacccggaaagggtagttttggagattcccccttctttcgctctgcctcttctttaccctaagcccaccacaggcctgtccgcgcgccaggcccagccgggtcgtttggctttgcaggcggccacccaggccggccggcttccacccgtgtccggtggcccagccgcaaccccgatcccaatccacatcgggcctccctgtcgccccagacggcggcttttgtgtattggagagaggcctggcctgagatatccgagctgacaccagtgatgtttcacattacacatctccgccgggcccagccgtgtaatccgctttttctctttttcctttcattcttgatttcctttttatcccccttcctctttgcacccgactgctataaaaagcacgcctcactcccacttggctcgacaagcagccgccctggaaggagaggcagctgcaaggagagcccagcgccgcggctacaaagcactagggtggagctgcggaatagcgggcggggtgggagggcgttttcgaaggatcccagaaaacccatagactctgtctttaattacttgccatttctaccctaggccatctaaactttgctcaggcgagaagagtacgtgagaggcccgttcccttgatgtgcaagagagctaatgaaagactgaccttgctcaaaaccacgccgcccaggacccagctctggctctggacagttaaactaaaaccattttcaacttcttcccggccttttatccaccagcatagcctcatgccttgcacaaatgccacccagagagtgtcttcattccctctgatttgggagagcattttggtctttattctttttatcgttgttttcttctttttgtttgctctgctctaaccgggggctttattttttctacccagagcacttaattttttttttttaacagcaaagcctctggatgccgcttgatttgcttgattctgttttctgcttccagaatcctaacaaatttggaatcttccaccgaccagcataaaccaggacgttgctattgggttatttatttgagctcatttttgccaatccataaagtacagatttgctacaaagttaaggtaagccctttttacaaaactatgattataatttagaagagggggtgtgagtttcaatttccagagttcaactcctgagagaagataaataaaccaagcagaaaagtctttcttctttttttctttctccttctaagaggactagtagttgtgtattaaaactttgctcccggagatcacaaaactaggaaatagggtgtgtgggagagacctgaatggccgaaacaaccgtaaagaaggtgtaagaagcgcgagcccaggagggaaaaagctgggccagggccgggacaaaggtttcccagggagggccaactcttccgtgtctctggcgggttttccttgttaaaggctcacaggttggagcctgttcgcggctcttggcctggtagggattttattagctctgctctggcaactgcaagccaggaacacaatgtcctgtgcaggggattgcccatgcagcccagctcgtgagatcgcgggatggcggggcagtgagccggtgccgctctgggagcctgagccagggcggcagtcctgtcggcctcggagagggaactgtaatctcgcaaccaggccgccgcgaggccttctgcctttgcaaagctgcgccccaccggcgccctcccaggcggcgctgccttccacattctctcctggtctacttggcctgtacctccacaacatcctccccccatccctcccagactccgtgctggctcctacccggactcgggcttccgtaaggttggtccacacagcgatttcttcgcgtgtggacatgtccgggtagcggttcctctggaaagtggcctccagctcctggagctgctggctggtaaagtgagtccgctgccgcctttgccgcttcttcttagacgggtcctcggcgcccacgtcctcattcttcccctgctggcttttatctttctctgaaaacgaaacacacacactttcccgtcagcatgcccacctgcaacgcggacgccaactggaccggcggcagaagccgtggaagagctgggctgcctggcgccggaggagggtgcgcgcggcggctccgggccgcgaggagcgctgcgcctgtggggtgtgcaggcgcaagtgtgggtgtccgcgccccatttcctcccctcccccagcgccgcacgttttatttacatgtttatctcactgcagcggcacattcacttttatagcctgtgctttcaagtatatttatacacctctgcgcagacacaccaaatctcctgggacgcgcacacgcgcgtggtttacagacccccctccccctcgcagaaagctcagatttccatgcggtttgggaaggctaggaaaagatgtggggattcggttgggcaccgaagttcgccggccctttcccaaaaaaaaaaaaaaaatgcctcttcgcgaagggcatttctgagtggtttcaggcaatttcctaacgagtggagctcctcgggagctgaaagccgagaggaaaacagggacagaggtcggcggcctctgaaggtcctcgaatcaagatgctgggatttttgtgacccaggaaacagaagggaggccagggtacgaatagagagggcggcagaattgctcgcgcccttagcgccccaggagccgggccggtcgagggagaactaaagggatgcggggtagtcaaaattccggctcccggaagttctgcggggagccaggcgaacgaccactcccaccacgcctccccccggaggggctgacttccttggggcgagagggagcgggtggcgcagagcagctgagcgggaatgtctgcagggcggcgcggcgccttacctgcggcctccgggctggaggtgtcggagatggtgtgcacctccagcctgtgcttggaggagtccagcgaccggggctgaccgggagccagaaccgaagccatggctaacggctggggatggtgacaggaagatgaggagacggccgacagcttggtccccgctgctcggtgctccaagtgaagcgggcctttcatgcagttcatggacgagggagcgcgacgctctactagtccttggctactgccccgccgagcccccgtagccgccgctgcccgctccgggtcgcgctctaggcgcggagtttccccgctgcggggagagccaggggacgcaacccccgccgagttctcaagccaagctgcccccgtctcctccggaaggctcaagcgaaaaagtccggagacggaaagtcagcgggcaaacgaagacatgggatgtgggcagaagggcaccactcagagcgtctttagggagcaggcttccaagctccaaagcgaaacaagagtgggcaaagacccccttcttctctccctccctcccccaagaacccctccaataaggaaagctaacgccgaccgcgctctgcccgccccccccccacgcggcagccctgacagagaagtgtcaagagtgacagggacaggtaggtgatattagatcccctgcggcggcagcagccgctgcagccacgacgcggccctctgagcgcaccctccgcaacgcgcacacgcacacccctcgggcggtcgaacaggagccgggccttgccgcagctcagctccaggcacccaggcgagcgacggaccagatctgcggctccgcgcttccctgttggcctaacatcttaaaaccagaggcgggcttcctggtgccgagacgtcactccgccgcggccctccccagccctctccgcctccgcctcctcccagacccttctccgggtgcgactgacgtggctccgcaccaatcaggacgccccgagccgcggtggagggactgtcctgcctgcacctatcagcagtgcggggccgggctactgcctcgccgtgcgcactgggtctacacaggcaagctcccgggaattcagctcctgcccagcccaaggcgatccggcttttagtacgaacccaaaggtgaagagatgaggctaggagtcgaaggcttgggagaagagagtggaatggtcaagaagagaaaggtacaaggatcaacaagacacccactctttgtgtctcactacatccatttccaatcccccaccccatataaaaaggagacacgttacttaaaactagaaaatttgaaaaacagcaacaaatcacctctccgatcttaaattttccaaacagcctgtcaagtgaatgctgcgctaatctgaagaagctttaattgcaaagaagacagagccctgaaaaggcaggctaataaattagaaatcgagaagcaaatggacccgtcaaaagaaaattaccttgactttaaacgaacaactgtttggtggttcactctggatttatacaagaataaaaagtcgcctcagatcacgttctctgtgatgcttattagtccccagacagaaaacacacaatagaagagaaaccctaacccagcgttttcaaaatgctgaaagcttatccattctacttaacgttgattaagacacatatcctagatctttcaaattccttgtacactgtattaagctcgtcctaacccgagagagccacgctttaaattcgactctcttgtttactttattatcaatcagatttaaatccataaagcctgtagaatcaacaaccttgagctaattatatatgaaatatgccttaatgaatttccatacaattaagaatgttgccaaataaccaatttcaaggataatttttaacagtcattttcttttcccagtgagctcaaggctgtcttgagccattaaagtccaagcaggcagaaggggtgtgtgtgagctaagggcgaaaagcctagaactgcgctcaactagcaaaagcaaaaccttatttatataaaacaaaaaaaatcacctttggagacatcaactctttatagcactgtttccaagcaaatttaatttccaaagaaattaaagaaagaaatccaaacatattcaaaataatttttgaaagtccttttgtcccccagcataggtcagctggagaggacaaactaatctcctctgggtttctgcatgggcgattgttttactatggagttagtgttatcatctctgaatgtgtatttgtttgacattacagtcaatgatttgcaatgccagcatgaagtatctttaaaacactccctccttgtccttgttcacaagattgggaaacttattcgatgtggaacaaagtggatgaagcagactacaaatatatttgcaacttatgtgtctctcctttgccctgaccacccccaaaccctatctgcaactcctccccattttaaacttgcagtccaaagacgcacatgagaattgtttttcagtctttcttcaccagtatcatcccactttaagaataatttagctgcaagggaggaatttcttcatagtaagctttaaatcagcatttctgcttttaaccttttattccactttaccccattccacacatacagacacctgctcagagtaaaacacatcctcatgtgacaggtctgcattagctgaggctcatacatccagctatattaggtcctgcaatcttatcactaaattatacacattacactagcagcctgttggtaaagaaggttaaattaatttacattctgctcattatctggtgcttaaatgacgcattttatcccggagatttggcggagaatctccttctcagaccccacagcgtttcactgaagacaatgctcttacatttgtagtggtttttaatctgataagactctaatttgcttaagtcttttaaataagggttttaaatgtttctagccgttttcttattgaatttcctctaattcccccaagatcataaagtatatgtgtaaagtaaatatttcctcccattgcactgccagccgatgacctataactaagtcaataagaatccagctcttttctgctgaatgtgtttactaatcatattccagtttcttcttttaaacctcagaatagctgtggtccccacaataccatgccccttaaagcttcatttcatgaagggactccatcacattaaagaatgaaaaaaatctccactgtagttagtatacacagcccctcactccttgtttttcaagattcaaaccccagagctgcaaatatttttggaagcttgggtgttaatgccttattttagaaagccgagaagccccacagagccatatagatttctaaacccatctctcataaacccacagaattttgataaaagctctggtggctccactctaccgatggaactttcatcacgacaaatatacatgtatgaaggacctcaatcagcctccaaagtggttgaaaaacccaagggcacgtgactgctcctcatagtgccaacgtgtgcgagatgttggaagcactggggatcagcagcagcctagatgcctaaaaagataaggtgtcctaatttgtgtggacccattgaagtcaagtggtgaataaagacaattatctagataattcagattaaagtaaaagcaaaaccatatctatttgtatatatatattcacatccattttatattacagacacacacacgcacacacacactggctctgtaaacaactgactcaaagtgaggattttctttgcatttttccagcaggagtttcaacattctcctaatctcctaatcactttacacactcacagcagcggcgattgggtgacattcttctcaggccccctgtgtggcaggacaccaacatgataagtctgcatggggaaaaggaggtatgtggtgggaactaagaaacactgtccagtgaaaactctgtggcatggtggtggttgattttggagatttaatgtacataagacttgtgggtgcacaggcataggcagcatggatgagaaaggggccagaagaaaataaatcttatgcattttgtgattccagcattactgtgacctctggctaagttcttcttaattggttttagaaattactatgagttcagcctctaacacagaaatttccaatacggagaacattggtgggatcctggtagggaaactagaggtgctgcatggctcacgtggggcaaagaaggaaagcccagtgccggcgtgaggttttgagtctgggagacatcaggggttgtctcgattggggtttcttgcttattctttcaaagaaagaccctagaggagggaaatgtgtgacatggggtcagccgtgttttgtgctggtatttgccaccgattaccagtcttaaagtcttatttaatttcacactcttcagtgttagttgtgcaaagtccctctggccatggcagtgagcggttgggctgtgccgccaaactctccgtatcaatctggcctgggactcaaccaagtgatctctgacttttggaaagagtctgtcttcagagttcacccagaagatggcttaattagacatctccctgagctgttaggccttagacgggtgggagtcctgccctgcccaagctagctcaaggacgaggcccgcctggactcagcttggagccacgtgatgggcgtgagtgtgtgagctcctggtaaggcgcagaggtcagatggagaccttgcatcctgcccgagaagtgccccaccccctccaatatctggcttttctctgcatacaaaccaagctgaaaacagtccactacccaccacccctcatagctatggaaccaaataacccagaaattaaaagcttcactgtagctgtccttttccccatttcctaaatggaatttaaaaagctctggcttgtcaaaaggggaagattattttctgaattggaagtctgtagatatattgagcaacagccaccctctctgggtccctgcaaatggtacccatttttccaacccacagctctagctgctcaaccatttgagatttggggtaactacctgggggaacagtgttcagatggcagtgggagttaccacctcacagtggcctggggaagagaagagaaagagattagaggagggggcatttgctaaaatcactcaacgaacatgctgttaatgcttcctcacatttgcatgttactgccacagttttcctaggtgtcactgagtctccagaaagcaactacttgccgaactaagtaaaataaggagaatggtatagcacatgtgtttggagaaggggaaggaagggtggaatatgaaattgagcatagatatccaggtcaggaaagaaggaagtggcaaggggctaaatgaagtcccacccccccgccacttttttaaacaacagttggattaaacactcatctgtcttcctctttttctcttttcttccctctccagcttatgtccatctcccttttctatttcttttgctctcccttctttctgttttctctctcccagacatgctggtagctaacattcagcattagttgtcatggtgaccataaatcacctaaatccaaaaatacccacctataatctgagatgaagcttttatttccctagcgaaataatattttaaaagctgtcagctgacaaaaaaaaaggaacccacctcattgtagtaacccaagtaatatattacttctaaaggtttaaattaaaatgtcagcctgttaaaaacatgctggtagagtcttggacactcttcccgtcagatcctcacaaagaagttgactctgtcattcctggtcgctctttaacatacacacacaattctgtatgctctctccctttctattaatttcttcctcccctaccaactactttagttctcaaagactctacgcattgggttctaaaagaaaagaatctcctcctggatcagaaaacagcctcattggttgctgaagtgaaagatgtggggtttagggggaaaggttattaggaccataatggcggtggtggtaggaggtcatcctagaggagttaagaagaaaaaaaaatgcagggagaaggactggaggtggaaagacagagtaacagaaaactgagctgggtggtcaggtgctgcggtgaagtctagccccgaaatgataggtatatattctcccactcctgcctttttctcctctgagagaaaactttcctagtcagagaccctggggggtaggaggcgggcaaacgccgctgcagttgggccttttgtcttctaccttgggcttgttgtcttttgcctatcttggattacagggtattcgcctagatgaagagccattaattacccattcagtcaatattaggaagacgacaaagctttttacataggattaagaagacatcagattgttccattagtatattcctgctcacgaataagctttcgttatacatttccttttcccccgagccgctctaacaactgctgcatatattagcagcgggcggtgaggaataacagccgaaccagccttaagaaactttgtgtaccgagtcagcagcgaggaacgcgacctgtgaagaccacttttgcgggcagggatccgcagggactgactactttcggacaactggtacaatttcccctgggggtgaaaaaccacaacgcggcggggcacctttcaagtgagctgcagatttgattggcgcggggggtgggggaggggaggggagaatgggatggcggaggtcgggcggaggaaagaaaatggaaaactctccttacccctatttcgttgctttttcctcaagcctcatgcccccttcggcaagcacctgtcccttccgcgctagccacagctagagttctccacctttctctacacatccccttctttctgacaaggctcaggaccttggtcaccaccccacgcaccaccattttgcgttttgctagagacggtctgggctgatctcgctggtgtccatgttaggattaaaatcccttcatgacggcgaggaaaactgtattatttgctttcagggggtacattaggagtccacgtagtatatgctccgcaaacattcgctgattgaatgagaggcgcgggggcggggcggcggagaggggtctgcggccgccaaggccgccagggttaacccaggtccttcgaagaaggttgggaccgagctgctgccgcgtgtgaaggtgtgtgtcgcggctgggggtgccacaacgggccatggagcccacctcccagagggaggaagtctgtgcacaccagcgacttgggtcgaacactttcagcccatccactgggccaaagctatttaacaattaatgcgttcctgggggaggccgggggaagtacccgccccttgtcgggacgcaaagccaggcgtcaggtccaaaggggctgcagtgcagtccgatttcagcacggaacccagagccgccgccctgaaactttttaagccagtgacagaggagggtcagtccgtcctcctcctgagggtgaagaccattttatgagttcctttcaggacccccaaagcaagaaaagccaaagaaaggtctctttgctctagggggtcgttctcagctggcttctacaccatcttctgctagctgcgtccaccctctctcaaacctctggcttttaggggtctccagtcgctctcgtgctacccccgtttcggggttctatccaggctgggcatccgcccaatggataccaaggagaatgggactcactagggaaggaaggcagagcctggactgcttagaggtggactctgcctatacagaacgtttagtctttaacgaggacatggtatttttgggcggcgttgggggcagaggcggagagggtagcgtaatagactatcacggccttttgaagaagtcatatgtcattgtgaactttttttctctttaaaagcaaagaaaaactttaaaaaaacacaagaaataaatccttttgttttacaagcaggctgtggccaggactttggatattccataagttaacttaaaatcagggaaggataggtgctctatccttcagcagtgctatagctttgcccactcgtgtgatttttttttgtcgtctactagttccttaaaagccaattaaatcaagattttcagcatttcttcccattacatgccttttcttaattaatggcattaaacgtgtttaggcagcaatttttcttttcggtcaaaaagtagaaaaagacatattgagtgtaggggaagagcctttcacgtgcactaaaacaatgcgggcctgaaagtaatggttaagaaagcaatcattcattatttctagttctttatgtgctagttatcaaaagcgaacgaccaggggcgcctgcgcggcttgtgactggcgcaagcagaactcctctgctcttagccgtttcctgctcacctacgaggaccccatcctaccgtaggcttctccacctgtcctcagaatgcattccccatgtctaggaaacctggggtagggactgggggaaggagacattttgcgtctctctggctcccagcacaataagaaatgtcagcctcggcttggcgactgcgagcccgccccgcgcgaagcgagattggggagctcctctagtctggccggagccagggctgagcccgcgcaaagcattctccccagaagtcatcgctgctcttgatttttaaccatatcccaaacatactacggtctaataatttatttttactaccgattatcaaacagaagaagaatttaacacaatctaaatgacagaaatcacgcgacgttatctctgcattgcccccatttaaccccatggggttaatcccggacaagtgagcgtttaactggcccagcagggcgactggcggtgtagtgcagtgtccgggcgtgaagcactggatcgctttagacgcttcatttcaacaaatgatcattttctctcagattcacggggaaactccaatgcaagacctttgttttcttcttagtaatacggtttgtttttttgaccggggtcccaaatcgcccccctccatcccatattacatttgtattcttacactttaatccggaaagagggggttaggggccgagggctgtgggggggggggggctttatctgctcacattatcaaaggtcaatagcctcctaaggctaggcacttatttactacggagccaggaaaatagaggaacagtaaatttgaggggtttttttccatgtatttgaaaagaaaggcatcttccctcctccatcccttacacccccccctccgccccatagaacaagtttcaattcaggaaaggcctgtggcgtaggttggagactttttaactttttacataagcctgtagacctcctctggcaatgtccctcgattcctccagagtgaaaccagccaatcaagcaacgacatcgccaaaacccaaggcctggcaaccagtacttcaggcaggttcgcgtcgacaggggcaaacctccattctaccctgggctgctaagcacagtggctgccctccagctcttcagggatgttgtcggccccccgcccctcccccaaccagccaaagcaaaccttcgccaactcaagtttcccttgcttgcctcccgcgatgaaccgcgcacccacaagtctgggtggggcgtggctgagagcctgagtgactgagtgggccctggtggtgctgcgcgcagcgggatataacgagcgacagaggtcgctgttggaccacttttccacgccagcctagacgccgaggtttgctggagcgtgcgcaggggatcagaccacagggagcgagcgagagggagagagaggtgttgggtctcaggagtgcagtataatttggggaaaggaattaacgtccctgggacggctgcttcccgtcccacccagaggcggagtgtctaagttcaagcagcaggcgcgtcaggtctggcggcctcgccttcttgcgcttcgcccgaggcccagagtcccggaggcgggtgcccagcgcgcggcctgcgcttccctcccggccttaccattggcgccaggatgctgccgcgggaagaacttgctgctggctgcctcttttcggctctcaggagagtccgtgaactcgacctttttgatttcggagtctttggagaagagacattcaacggccgccggctgcacgcctgggccacgcgcgcgcccgccccacgtgcggagagaggcgtcccggaccgcggccgaaaggagccggggacgggaggagggggaggggcgaggcaggccggaggagaaagagggacaaagagcaaagacccagttagaggaaagagctgacggcatccccgtcccccggaccctctggcaacccggggcaggatggcgtactctttctgcgcctccccggttgccggcggttccagccgggaggagtaggctggggggcttcggtacacagcgcgccgctgcttcctagtccaccgccccgccacagggagaggccactggcgatttggttctgatttccttcctgcccaggctggcccctcggggaagcgcccctcgctggggcctcgccgcagggccagtgccctcttgccgccctcacgtggcgcggcctcccgtccgatgacccgggcaggagaagggggttcttacctaattgcacacacgccgacaccagtttgcggcagttggtctccattccccgttatctgcaaaacagaagagaaggaaggctgtaagaagcggcggccgtcgagtgagcagggctcagatgagactacgttacactagctgttaggcgcctactgtgtgctaggcttcaggcgtgtttttccgacctgacagcctctggctgtgcagcagcatctccagcctagcctcgggcccaggacatttatttatcaagaggggatctccctccagagttgccgcaaaagtgcccagaggttagaggactataaagccacagtgtgctggggaggctgtggacctactttcaagaatctcggtgccggggttaagaactcatctgaacgcaatggcagcgggagtgggtgggtggagaggacttttctctttgggaagctgtatgcaaagaccacccttcagtgcttgtccattagttggagctcggtaaacatttgtagaacatcagcgccaacgtgcccctgtcctagacagcagtttccctcggttttctgtaattctgaaacgaacgggctcttggcctagggtgtttcaggagcgagctgagttcgggcctctcatccatcaggagccacttttccatatttagccacactttctcctagagatactaacccggccatttatttacttactacaaataattaccctaaagtatgatttaagatcgcagaggagagacactgggtggactgagcgagactgaggagagcagggcaaacgttcttggagggtttactgcccgccaaggacggagaaatagctctggtataactgctacccagttctccccctttctctcccgggcgagccaaatctttttcacgttttcaaccacaacgcagcgagccaagcatttaacgcgctcccccttctctgccacaggcaagccgggagaggtgggtctcgaggggctccaccgggtgggtagaagagccgcggttgctttaaagacaagaaaagaaggtccagggctccctaggccccttcgatctcagcgcctgcctctctccacgctaaccagggtacgccgacgaccggagggcccacttcgcgcgggtgcggggatcggggtgggagcaagcgttgtcgggttggcggaggcacagaggcggggcagggagctgcgggcctgcctctggcctgagtaccgtttccctgcgtctcggcctctcctgaagggagctgggccctggggagcctctggccaaggtcgctgcctacaggaggggctgcccggcgctgtggcgtggggatccagggtggggacggccaggcggttcccccacccgctagcgagaacgcgggcggggactctgccgattcgatccttgtgggcccgtgggcccagaagtagcagtttggcggctccagatttagtgattctgcagtaaaatcacaggattagtccctgattgagatgtctgttcgtgagatattacaaaattcattatcacagctttctactaactcgatatgaagtaacacagatgggattttattagtccagacttcaaatgtttacttatgataatttcggaggaaatttgcatgtcatcatcatttcgataatcttttttttttatatgtctgaactggctgcattattagctggtagccggagcactgcagatggtaactgcaaatagtttttatttatttattttttttaaagaatgaaatatacaaaagaaaaagattgcgttgcttggtgtaaagtcagtcaattattacacattcttccccccacttccccgtgcttcagtgctgaagaccaaacaaagcaatacaaaacaaatcttcaagaactcatagagctccactctaaggactgaaaagaaggtcaaggcgtgttccttagctcactcctacatgtccttgtgacttggagatttattttgcagtcaaaatgagccttgagacttgcatttttatgcttcatttaatgaccaggcctactagaagaactgagtctaaataactggggaagataattttttaaaaagagacctccaattcccgcctgctgatccttaaacttgccctatcaagacaagtcttctgtgagaaatttggctgccagacttcggaactggcttcaatggctaatctcacaaattgagatgggagacttttcctgatgggaggtagttctcacccccaaagttcatgttctagttggaatgtatatgccaaggactcttgttttggccaacttgggttttatattgtgagcacacaaaaagcactacacggctaacggaggacgaggaaccatggcaaagcaggcaggcaagccctaagaaataaaacaatttgctaaaaaataatttctgatgactaccgcaagactgaaagtgcaggaaaaatacagttcgaataatcccagatcctttcacatttccccccttttcatacactttgttaccccataacaaaatctttaatggaaagtttaaaaataaacagcacaggaacatgtgttttaaatgaactaaattgtgaaattagccagtaaattaatttgtagtaagtaattatttaaggaaattaaaatactgctcagttcagttctgtattttaccatgtgtatgcgttctttacaaccaattaatataagtgctttaggaacatttgaagacaaacacgcttaacttaaggaacaaagcacctaaataatttaagtgtaattttgctgagttaaagtaaaacattccacaaatgaagtggctatttaattttttagggaaagtttggttattgaaatgttgtatgctcatgttacatcaacaaaaatcttcaatttattttgcttatgtgctttgttttcttgatattattggtatttgaattttagatggatttctgccaaaatgatattttgtgtgataaaagcatctttagttttgattgatagactaaaacaaatgcaaggaaatttctttaaatcagattaatttttcataaaaatattttagaatgtatgaattctgatatttacatttataatggtaaaagttttttccgtttagtttagtaagacaatactcacacaaaagagtaaaaaaaaatcacaccaccttatgatagtttgatttctaaattgcttaagaaagtaaagtggttaaactggaaaagaggaacatatttcggaggtttagaatcgaaaatttttttcttaatctccagctggaaaataattctctgcatccatttaaagtgtatctcctgaagtgccagattggagttgactggtgatcaatttaaaggagttacaatccaaagaaatggtgagagcttggcatccaggcctggctcccaggtaattcgcttgggcctgagaggtcactaactgccagttaagatggaatctttttcttttcttttttttcccaatggataacaatgggaagggggctaatcttccagtagctgaaactttgtacccagccctttatcttgagaatgctaatccttggcccgaggatttgttcctgcagtgttggcaccgagatttaagggaagatacctcgttttaaatgccagccacggtctggcttccctctcgacttcagcaccctgtagattgttagtgtctgtggcgggggacgaaaggaacagggctttgcaaggtctgtttgccgactgcgttaccttgggcgaaacttagccccaaaagccacaaatcacctacggtgaagattctccgaagtggaacaaatttccagactcgcattatctcacatccctgcgggatagatggcctccacttaccggctaccgggagagagctgctgtctccgcgtcccactgcttcccggggcgatttccagcgagccgagcctccggctgcacggcaagcgcccgaaagccgggcctgagaggactgcagggctcctgagggtgccaagttccgaaggagtccacgggtgcactggggcctccgaaatctagccgccactggcagtttctttctgctcctctccagctttctcgctcggtctcgcactctctctcctctccctccctctcatccctctctcttccctctgctcctactccgtgtggggagtgacgtgacgtcagcagagattccaccaaactccactgcacagtggcgcgcgggcggccggccgagcccggctgcgcggctggcgatccaggagcgagcacagcgcccgggcgagcgccggggggagcgagcaggggcgacgagaaacgaggcaggggagggaagcagatgccagcgggccgaagagtcgggagccggagccgggagagcgaaaggagaggggacctggcggggcacttaggagccaaccgaggagcaggagcacggactcccactgtggaaaggaggaccagaagggaggatgggatggaagagaagaaaaagcaatctgcgccaacccggcagccctaataaatcaaagggggagcgccagggcagcggggagacagaaacgtacttttggggagcaaatcaggacgggctgggaggaagcgacagggaaagtggcccaagagacggaacaaaggacaatgttcatggggttgtttgggacgaggcgtgtggagtgtgggtgtgagcgtgcgtgtgtgaccttctttcaggcctgcagagttgaggaaagaggtcacagcaaagagggactgcggagggaggaaagtgagagaccggtagagggcgggagtggaggtgggcgcggtggggatgggagaggatgagtgaagagaaatctagaagaatggagtgagctagtgggagagggtgggagggccacagccgggagcgaacgagctaggcttgtcagctggggaaggccgggacgctgggcccagcttagctgggacaccgcgcccgaggtcaaggcgggtggaccaggcatgctgagagtgtcggcgcacaggtgggcacggccacgcactgacccagtgttcacgaagggtttgcactggacaaggctcagacgctcatagagtctagaatttcctctgctgtacctacattcaacaagttcaccctgggtcacggatatctcattttttaaaatgacgaggttaaggttcctggcgaggatggtattaaattgcacgggatagaagtgggggtgggggagagagtttccctcaagtccacatttgctcctgcaaagcaaagagtatgtgaaattacagggcatattctcactcgaaaagtgtgccttacttctgaaccctgattttctgatttcttgacttgagcaaagatgtgtattttggtagtgagcagaatattttggctctgtcctgcctctgagtggaaggactataaatataattcgcctggaggaccaggtgtgaaggcttctgccaggcatatgggacaatgttttttcaatctcaagggcatcctgttaatgtatgtttttggaaagtgccggaacacagccattgctcctggattcggattttcccaccaatattaattcctgcttgagagcaaaactcaggcccgctattaaaaagacatctctttggtccctaattgagaataaagttccctctaaaagttgtattgctcttcctaaatcaatataccaatactcgcaattttagaaatatatagtgactcgggagaatgtgcataaaatagatacgtttaaaaaagcttggcgcttaaaactaaccctagtcactatataggtgctgggctttccctacttttgggggctgtctggaacatgttatgtgttttcttgaattactccgtgttttgaattcatttgagttagcagtaaaaacaggcaaacaaacttgctcaatttgttttgagtgctaaatcccttcactttgaaatagctaacagtcgacagatggactcattttatggaaagggttagcctcttcagccacgaagaaaactgattagagatctacattttaagccatttctaacctccacgtaacatccgtgaaaactcaaactttctctctttacccagtggaaactcaaagcagtgttatttaaggggagagaaatgagggggaaaatgcccacgtgctgtttaattgtatttcctctctgactctgagaatttctatttctggtttttgaaatctcgccgaggcaagaaaatcaaatttcttcaacaagtcccacaactgaactctagttacaggacaccggaaagtgcagtccgagaaagacatcttcacctctgcccatcgacgatttttgcagcctccccattcctctgagtaatgggctaataatcctccctttttttcttttcattttgtagagattaagaggcgctcgtagcagaacggccttgccttcagctggtggcgaggataggcaatctcatggaaaagttggaagagaatgagaaaaccaaagacagaaagattcagagatccgcggagagacacagggagagggaagggagttgcgctgaaaagacgcaaagatacgcgcgtgcaactccctcccctttcaggtttcagaggtttgcaaaccagggctgagaggaaggggctcgggaagctcacgttcctctcgccccccttctgtctggagtctcgcccgccagaggctggttaaccccagtcccggccgccgcagacactgcgctgagcttttgggtcctcgccttgcccagcgccagtgcagctgaagtgagcagctggtgggaaatgcaaatggctcctggagaaatagaagatacagaatgattctcattccctcctcgagtgtgtggaaggagctggacatacgtttcacgctcctaatccttcttttacatttttagtcatactcctattaaacaactaattaatgcccagaatcaccagggaatacattaggcatgcaatcgtagaagcagggtgctggggggctacaaaccaccgagctgatttaagacgtggatttcaggtttttcccttgtcaaagcagtaaaggaagagcgggccttggcgactgtatttagattccgattacttcaaattagaagggggtggagggagcgtccaagcaaagcaagcaattctctgccctgcagatgtaaacaagattgtagcatcaaaggtactagctcctttagggctagatcgcctggactgggagcctggggaaggggagacactaaccttacgtatctgtgaatttcaaggatgttacatttttacataaacaactccagtgcggattctctggaatggggggagtaatacccccattctagaatattaaaacattttccccccaaagcgtatatcctttttatttcctcaaaattttgaactatgtctaaagataatagtcttccagtaaactggagcattggaccatttccttcaccctttcctaccgacattttgatgatctgattttaatgtgtggggggcacagggaattaaatacagcccataaaactaagcttagatgaaacagtgctggctaagtgggttcagataatttttaatgagaatctcaattacactcctccccccaatatgttgagacaagtgacagaaccgttagaatggtaatcaaattggaaagctcagggagaacaacaatttcgtgatcaaattggggcaaaaccgtggacaaatgtggggtgacctccgccaactccctgtcacccaagagtcaggatttgggaaaggtacagtattatttcagagcccgctgtgacgggctgtgtgctaccatttactttcttcactctggattatgatctcaaccctggcaagcaatttccccagctccttatctgacaacaagcgagtatgtaaataccaatggctagcgatgtttaactgcctcaaacattattgatttgttggctgttctaaattgtcttcctagtccaggtcttgtttccgaattgtctattctagaggtttgatccatgcctccgatgctacaatacaataattgtttttttaaaaaaggcatttaagatgaaccaattgatttgcatataaattaaaattactatgcgttgccgattccggtgttttataatcatttcgaaattagtacttaaccacttgagctaaaagaatatataaatgcctgtattgactcactaatgaattacccaattaaaacgtccgggcaatgctgggcgctggaaagattgttaaatcaagacatattacaggagggatatgaagattagaaaggtaacagaccaatatctcgcactcaaaacggagtttccggtgattcccagctttaattttggagcaggggtctttctcctctgctgttaaaaagattttgtgcttgtttgtgagtgagtgcattcaagtggaaggaacgctcccacggctacggtggctcaggccctttgctcggaccgggaccttacagttctaacccaggagcgttaaactctggaagactccgggccagccctggaggtgcgtggccccgcaagtcgccaggccaagcttgctttttctgtctgcccctccggcaggctgggcgcgctatggcagtgagctttccgcgcaaacggagagctggaaccaaagctgacatttaatagatatgctaactgagcacttaccttcgtcctgagaataggaataaaaggtagctcttcttaagagaggcggtgcaaaggcacgctataggagttcagaaaaggctggcggcgggaaatctgtagcctgggggctagtcaacatcccctttcatttcaagcacttattgatttgctgttgtcatctttggcgacgcagaaggacacttgaaagaatttctgatggggctctgatctgagaaaggaggtgacctgcccaggctcccaccaaattcttaattaccacatcaactgcttttttttatcccccacccgacctccttccttttgcttattcttaactttttaattattcagaaactcccttacctctcagtggcttcccttctgcagcagttttctattcgaaccttttccccgcctttccgtggtagggcctgtatattgatccctctgacctttggcacatctgggccctctgaaatctctcaatcttttcagatttgaggatggcaggctccaccctctccactgtgtgcacacactcagagatatgaaaacttacacagactgccttcaaacccagggtatctaacagatgttccctttccagttcgtctcttgatctgaaatgcctgcctgattccaacttggataccactctttcttgcccttcctttttcaaagcagtttggacatgtgtgcaagtgagcccagaacagctccacccatattctttaccaaactgtaaataaaagaagaactaatgaagtagattggcatatagattgcatcaagagcccgaatccccagtttctggattccccattcaactctggctgtcatctacattgacagagtcattctaagcagaggcccagagaaacctgcattgtgggacaacaggtaaagccacagtaaaaagtggaataattttaaagtcattttattagaatgtaaattgtatttctgggttttgttcgtaaccacctagttttaatatatacagagttagacaggaaaaaataggtcaacacagttattggtactagagaagacaaattccatgggctcctcagtgaaaagaagatccccaaagtctataatttttgatcatttaatttcatttataattgtgggaatgaataagacaccaactgctttatgtatttcatttacatcaaccaatttgtgtttccatcaaaagcagttatacagaatttcttttaacttctggtagcaagttcagaaaatgaagcttacagccaccctgaactggatacatctcttgagctgaccatttctgtaagtgcaggaatataatattgttcttctatggtctttttgcactcttttagggtttgcaagtccttatcaggtctgacatcactgtttgggtctacattcattacaagcaaatttgattactatgctgattttaaaacagcctatttggccagcataatcttagttttcaaattataaaaaccttttaatatacgaagttcccagtttttacctcctctagctccttgctcattcaaaacttctattttaattggtgtaagtaataataatttgtattactatttgtactccttttacttttttggagattgggctggattccagagagaacaccagcatcaccaccaccacaaacaacaaaatctaaaagtaaagcccttatttgcatgataattggtacttggaatgcttctgacttactcaatgccaccttataaaggtaccttgtaaactttcttggaatttctagcaagagcttgtagtaactggaacaactctctgggaagatatcctctttgatgggctttcagtttctggaggaatagattgagagcaattagggagggaggggacattggaaattggcagctacgtcagctgaaacaagcctgggttcagtaaggtgactgatgttgtggttgattccctaccccgagtttctctttaattggggcactgactcttcccactttgggatcccaaggcactcggtgtgtatgcagattcctcccttgtggtcttcaccatgtggtttcgtagcaggtctctggttcaatgatattttatagtcatagccctcatattcattatcatgatctcaatgtttaggcttttagtgtatttatattaaacctgctttattagtaagctggagcacacaggagagatgggggcaagtaaggactcagcagagctcaaattcagacatgtttaaatggctttgactgtgtaaagtgtggcaatgcttcctgctgccctagctttccactctaagcttcacatgtcctctggctaatgaagtgtgatataggccacatgctaggaataatagtatttgttgagaacaaagtgaactcaggaaacctggtatacacaaaatgcatc

SEQ ID NO: 2 - DNA, 28536 bp, Artificial Sequence
  chemically treated genomic DNA (Homo sapiens)
gttttgggagttaggtttttttcgttggatgggaaaaggaggtgcggaaaggggtaggcggtttgcgggttagcggggatgagagttgtggggagaacggaggagagagtaaaattattttttttggaagtttaagggaagggagttgaggtgagagaaatggttttttttaggttttaagttttcgtttttagtttgtattatttaagtcggggaagtgataattttttttaaatttcgatttttattttttgttttttgtagattgattgatgttttcgcgttgttttagtttagtaattaaattaggtcgaggtatattttttttatttagagtttttaaattatttattaatttgtaggagatttaaatttttcgggtaggtttcgttagcgtttttagtttttatttttaaatttttttttttagttttttttttttttttcgttttttggtttttttcgttttttattaggttgagaggcggagaggtcgggatcgttgggttttgcggttttagaagttaaagttcgttcgtggggatcgtttttttttttattgtttttaagtgaataatatggtgggaggtgattttcgtttaaagggtttttatttgttttttattgttgcgtgattttttcgtaagtcggaggtagagttttaaaaagaaagaaagtgtgcgttgagtattgattttaaatcggggatattatttgcgatttttttaagtggtagtagtagttttttgtagaataaagatttagtgttcgagttttaaaagtttttaggtaaagttttatttagatgaaagtaattaaggcgaaaaaataatgatattgtcggttttagaaaatcggcggttattttttgttttaagttttttagcgtggtgtttgttttcgtttattttggttgtgcggggggttgataagtataataaaagattttagtatatttttttttttatttttataacgatttttttagcgttatgaggatgaattttttgggattaagtggtatttgtttttatttgttatgaaattaaattttatatttttttaaagttgaaaatcgcggataaagaaggatcgggtgtttaaagtttatttaaatatttatatcgaatttattttttgtttagatgttagaggatggtagggagggttagggttgtaatttattttgatttgttttattgttttgtagtttgtaaattataattttgtataattttaggaataagtaggtttagaattaatgggttaatattatttaaaataaaatatcgggagtatgatttttgttttaattatatattgttcgataagatttaggaaattttattttaattttttaatttttgagttttttgagaaattgaagggttgtagttattagaaattatttttgttttttttaatgtttttaaaggatttggaaatagtaatgtaatataatatgaaggatttttttatttaagtttgaagataaacgtggaagtttaaatattttgaattggagatgtttttttgtaaatttattatagatgtattttttttgagagaaatgtatataaattatatatgtatatatgtataaatatttttttagaataatttgttagaggtgtaggttttttcgtaaaggagtaaatttgagagttattttaagttgatggatttgttaaattttttttttttttttttttttttatattatttgttagttataatggaattttttaggtttaagttaaagaaaaattggagagataaaattagattttgtagtttttttttttttcgggaatgtttttttttttttttttagtttttgatgaatggttattatttatttttattaaatttaaataaggattgttgttttgtatgtttaattaggtaggtagagggaattggtttgtttaggaagtagtgattgagatgttttggttaagttagtgatagaggaggggagaaagaatttagattaatttgtatgtagtatattttatttttatgaaataaaatatatttgttttatatttgttgaaaagtaaaataataatattgtacgaaatgttatatatagggtaggttgtatatagtagttttagaaatattattgtatttattagagaaattattttaaaattgatatttatatattttttataataataataatatgttagaaatatatagtgtggtatttagtatatatatttttttgttcgtaagcgaaaaattttaatcgtttttgtataatatgttttattttaaagtttaatttttaaaaatattgttgtgatattattaataattgtttttataaaattaatttgatatttcgatatatatatatttttttagttatttaaatgttaataatgttaaatttaaaaaataataagtttatagtaatgttaaaatgttatatttagttaaatatttgtttgtgtatgtgtttttgtaattgttagaaatatttgtagtgaaagatgttagatattgaggatatttttttgaaattaaaggagttttttttttgatttagtggtttttttttttttatatagtttttttttttttttttttttttagtgtttacgattttttagtataatttttagttttttaagggcggagttgttttattcggtaaggttttaggatttcggcgttgtgggtgcggtttatacgggtcggtttattgtatattggtaagtatttaggttggaggtcgggttttgtacgttggcgtagtcgaagttggagtgttgttttgtttttagttttaggttggttaggttcgagttatacgtgtttttataaatatacggaggagtcggcggcgcgtaaggataggtaggcgtcggtatcgcggaatttagcgacgggttatttaggttgtttaagttatttaggttgttgagattggagttcgggacgtttgttattgttgagggtattatgttggacgatatgtttatggacgagatagagttgggtggggaaaatatgttttgtgatgatagggggttgacgtttatagagttgaagaaggggaagtttttggtggatagggaggcggatgtaaggtttttggcggtttagttgttgtaggaatagtttgggtatatgtcgtcgtagggttgtatgagtttattgaattgcggttcgaagttatttttgtatagttcggtttgttggttgcgttttttttttttttatttggttcgacgatttttgaattaaatttgggggcggttggggtaagggagtaaatagatgttatagtgtagattattaaaatttttatcggaggttaattttcgttttttttcgatatatacgttagcgtatttatatattttggtttcgttttattgtatcgttttgtatattaagatattagggttagtttttagttattggttcgggtttttattaagcgtaggagatttggtttgttttggtttgcgagttgggattcggagttacgttataaattttagtcgaacgtatggagatttgcggacggtttgattatttagttaggcgtttttttaggtttaaaaatatttaatgtaaaataaacgcggggtagtaggtttttttaatttttttcggggtattttgtaaatttgtttttattttaaagttatagatttacggatgaggagaaggggttggaagggtattagaggatcgtttttttttttacgtaattttttttttttttttttgatttttattgtcgttttttattttttggtacgtgtttttttaatagggattaggtcgttaatattttttttcgtttagtaaaataattaaataaagagtaaaagattattttttcgttagttcgttaattttaggagtttggtatattaaatttcgggaattcggaaagggtagttttggagatttttttttttttcgttttgttttttttttattttaagtttattataggtttgttcgcgcgttaggtttagtcgggtcgtttggttttgtaggcggttatttaggtcggtcggtttttattcgtgttcggtggtttagtcgtaatttcgattttaatttatatcgggtttttttgtcgttttagacggcggtttttgtgtattggagagaggtttggtttgagatattcgagttgatattagtgatgttttatattatatattttcgtcgggtttagtcgtgtaattcgttttttttttttttttttttatttttgattttttttttatttttttttttttttgtattcgattgttataaaaagtacgttttatttttatttggttcgataagtagtcgttttggaaggagaggtagttgtaaggagagtttagcgtcgcggttataaagtattagggtggagttgcggaatagcgggcggggtgggagggcgttttcgaaggattttagaaaatttatagattttgtttttaattatttgttatttttattttaggttatttaaattttgtttaggcgagaagagtacgtgagaggttcgtttttttgatgtgtaagagagttaatgaaagattgattttgtttaaaattacgtcgtttaggatttagttttggttttggatagttaaattaaaattatttttaattttttttcggttttttatttattagtatagttttatgttttgtataaatgttatttagagagtgtttttattttttttgatttgggagagtattttggtttttattttttttatcgttgtttttttttttttgtttgttttgttttaatcgggggttttattttttttatttagagtatttaattttttttttttaatagtaaagtttttggatgtcgtttgatttgtttgattttgttttttgtttttagaattttaataaatttggaattttttatcgattagtataaattaggacgttgttattgggttatttatttgagtttatttttgttaatttataaagtatagatttgttataaagttaaggtaagttttttttataaaattatgattataatttagaagagggggtgtgagttttaatttttagagtttaatttttgagagaagataaataaattaagtagaaaagttttttttttttttttttttttttttttaagaggattagtagttgtgtattaaaattttgttttcggagattataaaattaggaaatagggtgtgtgggagagatttgaatggtcgaaataatcgtaaagaaggtgtaagaagcgcgagtttaggagggaaaaagttgggttagggtcgggataaaggttttttagggagggttaattttttcgtgtttttggcgggttttttttgttaaaggtttataggttggagtttgttcgcggtttttggtttggtagggattttattagttttgttttggtaattgtaagttaggaatataatgttttgtgtaggggattgtttatgtagtttagttcgtgagatcgcgggatggcggggtagtgagtcggtgtcgttttgggagtttgagttagggcggtagttttgtcggtttcggagagggaattgtaatttcgtaattaggtcgtcgcgaggttttttgtttttgtaaagttgcgttttatcggcgtttttttaggcggcgttgttttttatatttttttttggtttatttggtttgtatttttataatattttttttttattttttttagatttcgtgttggtttttattcggattcgggttttcgtaaggttggtttatatagcgattttttcgcgtgtggatatgttcgggtagcggtttttttggaaagtggtttttagtttttggagttgttggttggtaaagtgagttcgttgtcgtttttgtcgtttttttttagacgggttttcggcgtttacgtttttattttttttttgttggtttttatttttttttgaaaacgaaatatatatattttttcgttagtatgtttatttgtaacgcggacgttaattggatcggcggtagaagtcgtggaagagttgggttgtttggcgtcggaggagggtgcgcgcggcggtttcgggtcgcgaggagcgttgcgtttgtggggtgtgtaggcgtaagtgtgggtgttcgcgttttattttttttttttttttagcgtcgtacgttttatttatatgtttattttattgtagcggtatatttatttttatagtttgtgtttttaagtatatttatatatttttgcgtagatatattaaattttttgggacgcgtatacgcgcgtggtttatagatttttttttttttcgtagaaagtttagatttttatgcggtttgggaaggttaggaaaagatgtggggattcggttgggtatcgaagttcgtcggtttttttttaaaaaaaaaaaaaaaatgttttttcgcgaagggtatttttgagtggttttaggtaattttttaacgagtggagtttttcgggagttgaaagtcgagaggaaaatagggatagaggtcggcggtttttgaaggttttcgaattaagatgttgggatttttgtgatttaggaaatagaagggaggttagggtacgaatagagagggcggtagaattgttcgcgtttttagcgttttaggagtcgggtcggtcgagggagaattaaagggatgcggggtagttaaaatttcggttttcggaagttttgcggggagttaggcgaacgattatttttattacgttttttttcggaggggttgatttttttggggcgagagggagcgggtggcgtagagtagttgagcgggaatgtttgtagggcggcgcggcgttttatttgcggttttcgggttggaggtgtcggagatggtgtgtatttttagtttgtgtttggaggagtttagcgatcggggttgatcgggagttagaatcgaagttatggttaacggttggggatggtgataggaagatgaggagacggtcgatagtttggttttcgttgttcggtgttttaagtgaagcgggttttttatgtagtttatggacgagggagcgcgacgttttattagtttttggttattgtttcgtcgagttttcgtagtcgtcgttgttcgtttcgggtcgcgttttaggcgcggagttttttcgttgcggggagagttaggggacgtaattttcgtcgagtttttaagttaagttgttttcgtttttttcggaaggtttaagcgaaaaagttcggagacggaaagttagcgggtaaacgaagatatgggatgtgggtagaagggtattatttagagcgtttttagggagtaggtttttaagttttaaagcgaaataagagtgggtaaagatttttttttttttttttttttttttttaagaatttttttaataaggaaagttaacgtcgatcgcgttttgttcgttttttttttacgcggtagttttgatagagaagtgttaagagtgatagggataggtaggtgatattagattttttgcggcggtagtagtcgttgtagttacgacgcggttttttgagcgtatttttcgtaacgcgtatacgtatatttttcgggcggtcgaataggagtcgggttttgtcgtagtttagttttaggtatttaggcgagcgacggattagatttgcggtttcgcgtttttttgttggtttaatattttaaaattagaggcgggttttttggtgtcgagacgttatttcgtcgcggttttttttagtttttttcgttttcgtttttttttagattttttttcgggtgcgattgacgtggtttcgtattaattaggacgtttcgagtcgcggtggagggattgttttgtttgtatttattagtagtgcggggtcgggttattgtttcgtcgtgcgtattgggtttatataggtaagttttcgggaatttagtttttgtttagtttaaggcgattcggtttttagtacgaatttaaaggtgaagagatgaggttaggagtcgaaggtttgggagaagagagtggaatggttaagaagagaaaggtataaggattaataagatatttattttttgtgttttattatatttatttttaattttttattttatataaaaaggagatacgttatttaaaattagaaaatttgaaaaatagtaataaattattttttcgattttaaattttttaaatagtttgttaagtgaatgttgcgttaatttgaagaagttttaattgtaaagaagatagagttttgaaaaggtaggttaataaattagaaatcgagaagtaaatggattcgttaaaagaaaattattttgattttaaacgaataattgtttggtggtttattttggatttatataagaataaaaagtcgttttagattacgttttttgtgatgtttattagtttttagatagaaaatatataatagaagagaaattttaatttagcgtttttaaaatgttgaaagtttatttattttatttaacgttgattaagatatatattttagattttttaaattttttgtatattgtattaagttcgttttaattcgagagagttacgttttaaattcgatttttttgtttattttattattaattagatttaaatttataaagtttgtagaattaataattttgagttaattatatatgaaatatgttttaatgaatttttatataattaagaatgttgttaaataattaattttaaggataatttttaatagttattttttttttttagtgagtttaaggttgttttgagttattaaagtttaagtaggtagaaggggtgtgtgtgagttaagggcgaaaagtttagaattgcgtttaattagtaaaagtaaaattttatttatataaaataaaaaaaattatttttggagatattaattttttatagtattgtttttaagtaaatttaatttttaaagaaattaaagaaagaaatttaaatatatttaaaataatttttgaaagtttttttgttttttagtataggttagttggagaggataaattaattttttttgggtttttgtatgggcgattgttttattatggagttagtgttattatttttgaatgtgtatttgtttgatattatagttaatgatttgtaatgttagtatgaagtatttttaaaatattttttttttgtttttgtttataagattgggaaatttattcgatgtggaataaagtggatgaagtagattataaatatatttgtaatttatgtgttttttttttgttttgattatttttaaattttatttgtaatttttttttattttaaatttgtagtttaaagacgtatatgagaattgttttttagtttttttttattagtattattttattttaagaataatttagttgtaagggaggaatttttttatagtaagttttaaattagtatttttgtttttaattttttattttattttattttattttatatatatagatatttgtttagagtaaaatatatttttatgtgataggtttgtattagttgaggtttatatatttagttatattaggttttgtaattttattattaaattatatatattatattagtagtttgttggtaaagaaggttaaattaatttatattttgtttattatttggtgtttaaatgacgtattttatttcggagatttggcggagaattttttttttagattttatagcgttttattgaagataatgtttttatatttgtagtggtttttaatttgataagattttaatttgtttaagttttttaaataagggttttaaatgtttttagtcgtttttttattgaattttttttaatttttttaagattataaagtatatgtgtaaagtaaatatttttttttattgtattgttagtcgatgatttataattaagttaataagaatttagtttttttttgttgaatgtgtttattaattatattttagtttttttttttaaattttagaatagttgtggtttttataatattatgttttttaaagttttattttatgaagggattttattatattaaagaatgaaaaaaatttttattgtagttagtatatatagttttttattttttgttttttaagatttaaattttagagttgtaaatatttttggaagtttgggtgttaatgttttattttagaaagtcgagaagttttatagagttatatagatttttaaatttattttttataaatttatagaattttgataaaagttttggtggttttattttatcgatggaatttttattacgataaatatatatgtatgaaggattttaattagtttttaaagtggttgaaaaatttaagggtacgtgattgttttttatagtgttaacgtgtgcgagatgttggaagtattggggattagtagtagtttagatgtttaaaaagataaggtgttttaatttgtgtggatttattgaagttaagtggtgaataaagataattatttagataatttagattaaagtaaaagtaaaattatatttatttgtatatatatatttatatttattttatattatagatatatatacgtatatatatattggttttgtaaataattgatttaaagtgaggattttttttgtatttttttagtaggagttttaatatttttttaattttttaattattttatatatttatagtagcggcgattgggtgatattttttttaggttttttgtgtggtaggatattaatatgataagtttgtatggggaaaaggaggtatgtggtgggaattaagaaatattgtttagtgaaaattttgtggtatggtggtggttgattttggagatttaatgtatataagatttgtgggtgtataggtataggtagtatggatgagaaaggggttagaagaaaataaattttatgtattttgtgattttagtattattgtgatttttggttaagttttttttaattggttttagaaattattatgagtttagtttttaatatagaaatttttaatacggagaatattggtgggattttggtagggaaattagaggtgttgtatggtttacgtggggtaaagaaggaaagtttagtgtcggcgtgaggttttgagtttgggagatattaggggttgtttcgattggggttttttgtttatttttttaaagaaagattttagaggagggaaatgtgtgatatggggttagtcgtgttttgtgttggtatttgttatcgattattagttttaaagttttatttaattttatattttttagtgttagttgtgtaaagtttttttggttatggtagtgagcggttgggttgtgtcgttaaatttttcgtattaatttggtttgggatttaattaagtgatttttgatttttggaaagagtttgtttttagagtttatttagaagatggtttaattagatatttttttgagttgttaggttttagacgggtgggagttttgttttgtttaagttagtttaaggacgaggttcgtttggatttagtttggagttacgtgatgggcgtgagtgtgtgagtttttggtaaggcgtagaggttagatggagattttgtattttgttcgagaagtgttttattttttttaatatttggtttttttttgtatataaattaagttgaaaatagtttattatttattattttttatagttatggaattaaataatttagaaattaaaagttttattgtagttgtttttttttttattttttaaatggaatttaaaaagttttggtttgttaaaaggggaagattattttttgaattggaagtttgtagatatattgagtaatagttattttttttgggtttttgtaaatggtatttatttttttaatttatagttttagttgtttaattatttgagatttggggtaattatttgggggaatagtgtttagatggtagtgggagttattattttatagtggtttggggaagagaagagaaagagattagaggagggggtatttgttaaaattatttaacgaatatgttgttaatgtttttttatatttgtatgttattgttatagtttttttaggtgttattgagtttttagaaagtaattatttgtcgaattaagtaaaataaggagaatggtatagtatatgtgtttggagaaggggaaggaagggtggaatatgaaattgagtatagatatttaggttaggaaagaaggaagtggtaaggggttaaatgaagttttattttttcgttatttttttaaataatagttggattaaatatttatttgtttttttttttttttttttttttttttttttagtttatgtttattttttttttttattttttttgtttttttttttttttgtttttttttttttagatatgttggtagttaatatttagtattagttgttatggtgattataaattatttaaatttaaaaatatttatttataatttgagatgaagtttttatttttttagcgaaataatattttaaaagttgttagttgataaaaaaaaaggaatttattttattgtagtaatttaagtaatatattatttttaaaggtttaaattaaaatgttagtttgttaaaaatatgttggtagagttttggatatttttttcgttagatttttataaagaagttgattttgttatttttggtcgttttttaatatatatatataattttgtatgttttttttttttttattaattttttttttttttattaattattttagtttttaaagattttacgtattgggttttaaaagaaaagaatttttttttggattagaaaatagttttattggttgttgaagtgaaagatgtggggtttagggggaaaggttattaggattataatggcggtggtggtaggaggttattttagaggagttaagaagaaaaaaaaatgtagggagaaggattggaggtggaaagatagagtaatagaaaattgagttgggtggttaggtgttgcggtgaagtttagtttcgaaatgataggtatatatttttttatttttgttttttttttttttgagagaaaatttttttagttagagattttggggggtaggaggcgggtaaacgtcgttgtagttgggttttttgttttttattttgggtttgttgttttttgtttattttggattatagggtattcgtttagatgaagagttattaattatttatttagttaatattaggaagacgataaagttttttatataggattaagaagatattagattgttttattagtatatttttgtttacgaataagttttcgttatatatttttttttttttcgagtcgttttaataattgttgtatatattagtagcgggcggtgaggaataatagtcgaattagttttaagaaattttgtgtatcgagttagtagcgaggaacgcgatttgtgaagattatttttgcgggtagggattcgtagggattgattattttcggataattggtataattttttttgggggtgaaaaattataacgcggcggggtattttttaagtgagttgtagatttgattggcgcggggggtgggggaggggaggggagaatgggatggcggaggtcgggcggaggaaagaaaatggaaaattttttttatttttatttcgttgttttttttttaagttttatgtttttttcggtaagtatttgtttttttcgcgttagttatagttagagttttttatttttttttatatatttttttttttttgataaggtttaggattttggttattattttacgtattattattttgcgttttgttagagacggtttgggttgatttcgttggtgtttatgttaggattaaaatttttttatgacggcgaggaaaattgtattatttgtttttagggggtatattaggagtttacgtagtatatgtttcgtaaatattcgttgattgaatgagaggcgcgggggcggggcggcggagaggggtttgcggtcgttaaggtcgttagggttaatttaggtttttcgaagaaggttgggatcgagttgttgtcgcgtgtgaaggtgtgtgtcgcggttgggggtgttataacgggttatggagtttattttttagagggaggaagtttgtgtatattagcgatttgggtcgaatatttttagtttatttattgggttaaagttatttaataattaatgcgtttttgggggaggtcgggggaagtattcgttttttgtcgggacgtaaagttaggcgttaggtttaaaggggttgtagtgtagttcgattttagtacggaatttagagtcgtcgttttgaaattttttaagttagtgatagaggagggttagttcgttttttttttgagggtgaagattattttatgagttttttttaggatttttaaagtaagaaaagttaaagaaaggtttttttgttttagggggtcgtttttagttggtttttatattattttttgttagttgcgtttatttttttttaaatttttggtttttaggggtttttagtcgttttcgtgttattttcgtttcggggttttatttaggttgggtattcgtttaatggatattaaggagaatgggatttattagggaaggaaggtagagtttggattgtttagaggtggattttgtttatatagaacgtttagtttttaacgaggatatggtatttttgggcggcgttgggggtagaggcggagagggtagcgtaatagattattacggttttttgaagaagttatatgttattgtgaattttttttttttttaaaagtaaagaaaaattttaaaaaaatataagaaataaatttttttgttttataagtaggttgtggttaggattttggatattttataagttaatttaaaattagggaaggataggtgttttattttttagtagtgttatagttttgtttattcgtgtgatttttttttgtcgtttattagttttttaaaagttaattaaattaagatttttagtatttttttttattatatgtttttttttaattaatggtattaaacgtgtttaggtagtaattttttttttcggttaaaaagtagaaaaagatatattgagtgtaggggaagagttttttacgtgtattaaaataatgcgggtttgaaagtaatggttaagaaagtaattatttattatttttagttttttatgtgttagttattaaaagcgaacgattaggggcgtttgcgcggtttgtgattggcgtaagtagaatttttttgtttttagtcgttttttgtttatttacgaggattttattttatcgtaggtttttttatttgtttttagaatgtattttttatgtttaggaaatttggggtagggattgggggaaggagatattttgcgtttttttggtttttagtataataagaaatgttagtttcggtttggcgattgcgagttcgtttcgcgcgaagcgagattggggagtttttttagtttggtcggagttagggttgagttcgcgtaaagtattttttttagaagttatcgttgtttttgatttttaattatattttaaatatattacggtttaataatttatttttattatcgattattaaatagaagaagaatttaatataatttaaatgatagaaattacgcgacgttatttttgtattgtttttatttaattttatggggttaatttcggataagtgagcgtttaattggtttagtagggcgattggcggtgtagtgtagtgttcgggcgtgaagtattggatcgttttagacgttttattttaataaatgattatttttttttagatttacggggaaattttaatgtaagatttttgttttttttttagtaatacggtttgtttttttgatcggggttttaaatcgtttttttttattttatattatatttgtatttttatattttaattcggaaagagggggttaggggtcgagggttgtgggggggggggggttttatttgtttatattattaaaggttaatagttttttaaggttaggtatttatttattacggagttaggaaaatagaggaatagtaaatttgaggggtttttttttatgtatttgaaaagaaaggtatttttttttttttattttttatattttttttttcgttttatagaataagttttaatttaggaaaggtttgtggcgtaggttggagattttttaattttttatataagtttgtagattttttttggtaatgtttttcgatttttttagagtgaaattagttaattaagtaacgatatcgttaaaatttaaggtttggtaattagtattttaggtaggttcgcgtcgataggggtaaatttttattttattttgggttgttaagtatagtggttgttttttagttttttagggatgttgtcggtttttcgttttttttttaattagttaaagtaaattttcgttaatttaagttttttttgtttgtttttcgcgatgaatcgcgtatttataagtttgggtggggcgtggttgagagtttgagtgattgagtgggttttggtggtgttgcgcgtagcgggatataacgagcgatagaggtcgttgttggattatttttttacgttagtttagacgtcgaggtttgttggagcgtgcgtaggggattagattatagggagcgagcgagagggagagagaggtgttgggttttaggagtgtagtataatttggggaaaggaattaacgtttttgggacggttgtttttcgttttatttagaggcggagtgtttaagtttaagtagtaggcgcgttaggtttggcggtttcgtttttttgcgtttcgttcgaggtttagagtttcggaggcgggtgtttagcgcgcggtttgcgtttttttttcggttttattattggcgttaggatgttgtcgcgggaagaatttgttgttggttgttttttttcggtttttaggagagttcgtgaattcgatttttttgatttcggagtttttggagaagagatatttaacggtcgtcggttgtacgtttgggttacgcgcgcgttcgttttacgtgcggagagaggcgtttcggatcgcggtcgaaaggagtcggggacgggaggagggggaggggcgaggtaggtcggaggagaaagagggataaagagtaaagatttagttagaggaaagagttgacggtattttcgtttttcggattttttggtaattcggggtaggatggcgtattttttttgcgttttttcggttgtcggcggttttagtcgggaggagtaggttggggggtttcggtatatagcgcgtcgttgttttttagtttatcgtttcgttatagggagaggttattggcgatttggttttgattttttttttgtttaggttggtttttcggggaagcgtttttcgttggggtttcgtcgtagggttagtgtttttttgtcgtttttacgtggcgcggtttttcgttcgatgattcgggtaggagaagggggtttttatttaattgtatatacgtcgatattagtttgcggtagttggtttttatttttcgttatttgtaaaatagaagagaaggaaggttgtaagaagcggcggtcgtcgagtgagtagggtttagatgagattacgttatattagttgttaggcgtttattgtgtgttaggttttaggcgtgttttttcgatttgatagtttttggttgtgtagtagtatttttagtttagtttcgggtttaggatatttatttattaagaggggatttttttttagagttgtcgtaaaagtgtttagaggttagaggattataaagttatagtgtgttggggaggttgtggatttatttttaagaatttcggtgtcggggttaagaatttatttgaacgtaatggtagcgggagtgggtgggtggagaggatttttttttttgggaagttgtatgtaaagattattttttagtgtttgtttattagttggagttcggtaaatatttgtagaatattagcgttaacgtgtttttgttttagatagtagtttttttcggttttttgtaattttgaaacgaacgggtttttggtttagggtgttttaggagcgagttgagttcgggttttttatttattaggagttatttttttatatttagttatatttttttttagagatattaattcggttatttatttatttattataaataattattttaaagtatgatttaagatcgtagaggagagatattgggtggattgagcgagattgaggagagtagggtaaacgtttttggagggtttattgttcgttaaggacggagaaatagttttggtataattgttatttagttttttttttttttttttcgggcgagttaaattttttttacgtttttaattataacgtagcgagttaagtatttaacgcgtttttttttttttgttataggtaagtcgggagaggtgggtttcgaggggttttatcgggtgggtagaagagtcgcggttgttttaaagataagaaaagaaggtttagggttttttaggttttttcgattttagcgtttgtttttttttacgttaattagggtacgtcgacgatcggagggtttatttcgcgcgggtgcggggatcggggtgggagtaagcgttgtcgggttggcggaggtatagaggcggggtagggagttgcgggtttgtttttggtttgagtatcgtttttttgcgtttcggttttttttgaagggagttgggttttggggagtttttggttaaggtcgttgtttataggaggggttgttcggcgttgtggcgtggggatttagggtggggacggttaggcggtttttttattcgttagcgagaacgcgggcggggattttgtcgattcgatttttgtgggttcgtgggtttagaagtagtagtttggcggttttagatttagtgattttgtagtaaaattataggattagtttttgattgagatgtttgttcgtgagatattataaaatttattattatagttttttattaattcgatatgaagtaatatagatgggattttattagtttagattttaaatgtttatttatgataatttcggaggaaatttgtatgttattattatttcgataattttttttttttatatgtttgaattggttgtattattagttggtagtcggagtattgtagatggtaattgtaaatagtttttatttatttattttttttaaagaatgaaatatataaaagaaaaagattgcgttgtttggtgtaaagttagttaattattatatatttttttttttattttttcgtgttttagtgttgaagattaaataaagtaatataaaataaatttttaagaatttatagagttttattttaaggattgaaaagaaggttaaggcgtgttttttagtttatttttatatgtttttgtgatttggagatttattttgtagttaaaatgagttttgagatttgtatttttatgttttatttaatgattaggtttattagaagaattgagtttaaataattggggaagataattttttaaaaagagatttttaattttcgtttgttgatttttaaatttgttttattaagataagttttttgtgagaaatttggttgttagatttcggaattggttttaatggttaattttataaattgagatgggagattttttttgatgggaggtagtttttatttttaaagtttatgttttagttggaatgtatatgttaaggatttttgttttggttaatttgggttttatattgtgagtatataaaaagtattatacggttaacggaggacgaggaattatggtaaagtaggtaggtaagttttaagaaataaaataatttgttaaaaaataatttttgatgattatcgtaagattgaaagtgtaggaaaaatatagttcgaataattttagatttttttatattttttttttttttatatattttgttattttataataaaatttttaatggaaagtttaaaaataaatagtataggaatatgtgttttaaatgaattaaattgtgaaattagttagtaaattaatttgtagtaagtaattatttaaggaaattaaaatattgtttagtttagttttgtattttattatgtgtatgcgttttttataattaattaatataagtgttttaggaatatttgaagataaatacgtttaatttaaggaataaagtatttaaataatttaagtgtaattttgttgagttaaagtaaaatattttataaatgaagtggttatttaattttttagggaaagtttggttattgaaatgttgtatgtttatgttatattaataaaaatttttaatttattttgtttatgtgttttgtttttttgatattattggtatttgaattttagatggatttttgttaaaatgatattttgtgtgataaaagtatttttagttttgattgatagattaaaataaatgtaaggaaatttttttaaattagattaattttttataaaaatattttagaatgtatgaattttgatatttatatttataatggtaaaagtttttttcgtttagtttagtaagataatatttatataaaagagtaaaaaaaaattatattattttatgatagtttgatttttaaattgtttaagaaagtaaagtggttaaattggaaaagaggaatatatttcggaggtttagaatcgaaaattttttttttaatttttagttggaaaataattttttgtatttatttaaagtgtattttttgaagtgttagattggagttgattggtgattaatttaaaggagttataatttaaagaaatggtgagagtttggtatttaggtttggtttttaggtaattcgtttgggtttgagaggttattaattgttagttaagatggaattttttttttttttttttttttttaatggataataatgggaagggggttaattttttagtagttgaaattttgtatttagttttttattttgagaatgttaatttttggttcgaggatttgtttttgtagtgttggtatcgagatttaagggaagatatttcgttttaaatgttagttacggtttggtttttttttcgattttagtattttgtagattgttagtgtttgtggcgggggacgaaaggaatagggttttgtaaggtttgtttgtcgattgcgttattttgggcgaaatttagttttaaaagttataaattatttacggtgaagatttttcgaagtggaataaatttttagattcgtattattttatatttttgcgggatagatggtttttatttatcggttatcgggagagagttgttgttttcgcgttttattgtttttcggggcgatttttagcgagtcgagttttcggttgtacggtaagcgttcgaaagtcgggtttgagaggattgtagggtttttgagggtgttaagtttcgaaggagtttacgggtgtattggggttttcgaaatttagtcgttattggtagtttttttttgtttttttttagttttttcgttcggtttcgtattttttttttttttttttttttttattttttttttttttttttgtttttatttcgtgtggggagtgacgtgacgttagtagagattttattaaattttattgtatagtggcgcgcgggcggtcggtcgagttcggttgcgcggttggcgatttaggagcgagtatagcgttcgggcgagcgtcggggggagcgagtaggggcgacgagaaacgaggtaggggagggaagtagatgttagcgggtcgaagagtcgggagtcggagtcgggagagcgaaaggagaggggatttggcggggtatttaggagttaatcgaggagtaggagtacggatttttattgtggaaaggaggattagaagggaggatgggatggaagagaagaaaaagtaatttgcgttaattcggtagttttaataaattaaagggggagcgttagggtagcggggagatagaaacgtatttttggggagtaaattaggacgggttgggaggaagcgatagggaaagtggtttaagagacggaataaaggataatgtttatggggttgtttgggacgaggcgtgtggagtgtgggtgtgagcgtgcgtgtgtgatttttttttaggtttgtagagttgaggaaagaggttatagtaaagagggattgcggagggaggaaagtgagagatcggtagagggcgggagtggaggtgggcgcggtggggatgggagaggatgagtgaagagaaatttagaagaatggagtgagttagtgggagagggtgggagggttatagtcgggagcgaacgagttaggtttgttagttggggaaggtcgggacgttgggtttagtttagttgggatatcgcgttcgaggttaaggcgggtggattaggtatgttgagagtgtcggcgtataggtgggtacggttacgtattgatttagtgtttacgaagggtttgtattggataaggtttagacgtttatagagtttagaattttttttgttgtatttatatttaataagtttattttgggttacggatattttattttttaaaatgacgaggttaaggtttttggcgaggatggtattaaattgtacgggatagaagtgggggtgggggagagagttttttttaagtttatatttgtttttgtaaagtaaagagtatgtgaaattatagggtatatttttattcgaaaagtgtgttttatttttgaattttgattttttgattttttgatttgagtaaagatgtgtattttggtagtgagtagaatattttggttttgttttgtttttgagtggaaggattataaatataattcgtttggaggattaggtgtgaaggtttttgttaggtatatgggataatgtttttttaattttaagggtattttgttaatgtatgtttttggaaagtgtcggaatatagttattgtttttggattcggatttttttattaatattaatttttgtttgagagtaaaatttaggttcgttattaaaaagatatttttttggtttttaattgagaataaagttttttttaaaagttgtattgttttttttaaattaatatattaatattcgtaattttagaaatatatagtgattcgggagaatgtgtataaaatagatacgtttaaaaaagtttggcgtttaaaattaattttagttattatataggtgttgggttttttttatttttgggggttgtttggaatatgttatgtgtttttttgaattatttcgtgttttgaatttatttgagttagtagtaaaaataggtaaataaatttgtttaatttgttttgagtgttaaatttttttattttgaaatagttaatagtcgatagatggatttattttatggaaagggttagtttttttagttacgaagaaaattgattagagatttatattttaagttatttttaatttttacgtaatattcgtgaaaatttaaattttttttttttatttagtggaaatttaaagtagtgttatttaaggggagagaaatgagggggaaaatgtttacgtgttgtttaattgtattttttttttgattttgagaatttttatttttggtttttgaaatttcgtcgaggtaagaaaattaaatttttttaataagttttataattgaattttagttataggatatcggaaagtgtagttcgagaaagatatttttatttttgtttatcgacgatttttgtagtttttttatttttttgagtaatgggttaataattttttttttttttttttttattttgtagagattaagaggcgttcgtagtagaacggttttgtttttagttggtggcgaggataggtaattttatggaaaagttggaagagaatgagaaaattaaagatagaaagatttagagattcgcggagagatatagggagagggaagggagttgcgttgaaaagacgtaaagatacgcgcgtgtaattttttttttttttaggttttagaggtttgtaaattagggttgagaggaaggggttcgggaagtttacgtttttttcgttttttttttgtttggagtttcgttcgttagaggttggttaattttagtttcggtcgtcgtagatattgcgttgagtttttgggttttcgttttgtttagcgttagtgtagttgaagtgagtagttggtgggaaatgtaaatggtttttggagaaatagaagatatagaatgatttttatttttttttcgagtgtgtggaaggagttggatatacgttttacgtttttaattttttttttatatttttagttatatttttattaaataattaattaatgtttagaattattagggaatatattaggtatgtaatcgtagaagtagggtgttggggggttataaattatcgagttgatttaagacgtggattttaggttttttttttgttaaagtagtaaaggaagagcgggttttggcgattgtatttagatttcgattattttaaattagaagggggtggagggagcgtttaagtaaagtaagtaattttttgttttgtagatgtaaataagattgtagtattaaaggtattagtttttttagggttagatcgtttggattgggagtttggggaaggggagatattaattttacgtatttgtgaattttaaggatgttatatttttatataaataattttagtgcggattttttggaatggggggagtaatatttttattttagaatattaaaatattttttttttaaagcgtatattttttttatttttttaaaattttgaattatgtttaaagataatagttttttagtaaattggagtattggattattttttttatttttttttatcgatattttgatgatttgattttaatgtgtggggggtatagggaattaaatatagtttataaaattaagtttagatgaaatagtgttggttaagtgggtttagataatttttaatgagaattttaattatatttttttttttaatatgttgagataagtgatagaatcgttagaatggtaattaaattggaaagtttagggagaataataatttcgtgattaaattggggtaaaatcgtggataaatgtggggtgattttcgttaattttttgttatttaagagttaggatttgggaaaggtatagtattattttagagttcgttgtgacgggttgtgtgttattatttattttttttattttggattatgattttaattttggtaagtaatttttttagttttttatttgataataagcgagtatgtaaatattaatggttagcgatgtttaattgttttaaatattattgatttgttggttgttttaaattgtttttttagtttaggttttgttttcgaattgtttattttagaggtttgatttatgttttcgatgttataatataataattgtttttttaaaaaaggtatttaagatgaattaattgatttgtatataaattaaaattattatgcgttgtcgatttcggtgttttataattatttcgaaattagtatttaattatttgagttaaaagaatatataaatgtttgtattgatttattaatgaattatttaattaaaacgttcgggtaatgttgggcgttggaaagattgttaaattaagatatattataggagggatatgaagattagaaaggtaatagattaatatttcgtatttaaaacggagttttcggtgatttttagttttaattttggagtaggggttttttttttttgttgttaaaaagattttgtgtttgtttgtgagtgagtgtatttaagtggaaggaacgtttttacggttacggtggtttaggttttttgttcggatcgggattttatagttttaatttaggagcgttaaattttggaagatttcgggttagttttggaggtgcgtggtttcgtaagtcgttaggttaagtttgttttttttgtttgttttttcggtaggttgggcgcgttatggtagtgagtttttcgcgtaaacggagagttggaattaaagttgatatttaatagatatgttaattgagtatttattttcgttttgagaataggaataaaaggtagttttttttaagagaggcggtgtaaaggtacgttataggagtttagaaaaggttggcggcgggaaatttgtagtttgggggttagttaatatttttttttattttaagtatttattgatttgttgttgttatttttggcgacgtagaaggatatttgaaagaatttttgatggggttttgatttgagaaaggaggtgatttgtttaggtttttattaaatttttaattattatattaattgtttttttttattttttattcgatttttttttttttgtttatttttaattttttaattatttagaaatttttttattttttagtggttttttttttgtagtagttttttattcgaatttttttttcgttttttcgtggtagggtttgtatattgatttttttgatttttggtatatttgggttttttgaaattttttaatttttttagatttgaggatggtaggttttattttttttattgtgtgtatatatttagagatatgaaaatttatatagattgtttttaaatttagggtatttaatagatgttttttttttagttcgttttttgatttgaaatgtttgtttgattttaatttggatattatttttttttgtttttttttttttaaagtagtttggatatgtgtgtaagtgagtttagaatagttttatttatattttttattaaattgtaaataaaagaagaattaatgaagtagattggtatatagattgtattaagagttcgaatttttagtttttggattttttatttaattttggttgttatttatattgatagagttattttaagtagaggtttagagaaatttgtattgtgggataataggtaaagttatagtaaaaagtggaataattttaaagttattttattagaatgtaaattgtatttttgggttttgttcgtaattatttagttttaatatatatagagttagataggaaaaaataggttaatatagttattggtattagagaagataaattttatgggttttttagtgaaaagaagatttttaaagtttataatttttgattatttaattttatttataattgtgggaatgaataagatattaattgttttatgtattttatttatattaattaatttgtgtttttattaaaagtagttatatagaattttttttaatttttggtagtaagtttagaaaatgaagtttatagttattttgaattggatatattttttgagttgattatttttgtaagtgtaggaatataatattgtttttttatggtttttttgtatttttttagggtttgtaagtttttattaggtttgatattattgtttgggtttatatttattataagtaaatttgattattatgttgattttaaaatagtttatttggttagtataattttagtttttaaattataaaaattttttaatatacgaagtttttagtttttattttttttagttttttgtttatttaaaatttttattttaattggtgtaagtaataataatttgtattattatttgtattttttttatttttttggagattgggttggattttagagagaatattagtattattattattataaataataaaatttaaaagtaaagtttttatttgtatgataattggtatttggaatgtttttgatttatttaatgttattttataaaggtattttgtaaatttttttggaatttttagtaagagtttgtagtaattggaataattttttgggaagatattttttttgatgggtttttagtttttggaggaatagattgagagtaattagggagggaggggatattggaaattggtagttacgttagttgaaataagtttgggtttagtaaggtgattgatgttgtggttgattttttatttcgagtttttttttaattggggtattgattttttttattttgggattttaaggtattcggtgtgtatgtagattttttttttgtggtttttattatgtggtttcgtagtaggtttttggtttaatgatattttatagttatagtttttatatttattattatgattttaatgtttaggtttttagtgtatttatattaaatttgttttattagtaagttggagtatataggagagatgggggtaagtaaggatttagtagagtttaaatttagatatgtttaaatggttttgattgtgtaaagtgtggtaatgttttttgttgttttagttttttattttaagttttatatgttttttggttaatgaagtgtgatataggttatatgttaggaataatagtatttgttgagaataaagtgaatttaggaaatttggtatatataaaatgtatt

SEQ ID NO: 3 - DNA, 28536 bp, Artificial Sequence
  chemically treated genomic DNA (Homo sapiens)
gatgtattttgtgtatattaggttttttgagtttattttgtttttaataaatattattatttttagtatgtggtttatattatattttattagttagaggatatgtgaagtttagagtggaaagttagggtagtaggaagtattgttatattttatatagttaaagttatttaaatatgtttgaatttgagttttgttgagtttttatttgtttttattttttttgtgtgttttagtttattaataaagtaggtttaatataaatatattaaaagtttaaatattgagattatgataatgaatatgagggttatgattataaaatattattgaattagagatttgttacgaaattatatggtgaagattataagggaggaatttgtatatatatcgagtgttttgggattttaaagtgggaagagttagtgttttaattaaagagaaattcggggtagggaattaattataatattagttattttattgaatttaggtttgttttagttgacgtagttgttaatttttaatgttttttttttttttaattgtttttaatttatttttttagaaattgaaagtttattaaagaggatatttttttagagagttgttttagttattataagtttttgttagaaattttaagaaagtttataaggtatttttataaggtggtattgagtaagttagaagtattttaagtattaattattatgtaaataagggttttatttttagattttgttgtttgtggtggtggtgatgttggtgttttttttggaatttagtttaatttttaaaaaagtaaaaggagtataaatagtaatataaattattattatttatattaattaaaatagaagttttgaatgagtaaggagttagaggaggtaaaaattgggaatttcgtatattaaaaggtttttataatttgaaaattaagattatgttggttaaataggttgttttaaaattagtatagtaattaaatttgtttgtaatgaatgtagatttaaatagtgatgttagatttgataaggatttgtaaattttaaaagagtgtaaaaagattatagaagaataatattatatttttgtatttatagaaatggttagtttaagagatgtatttagtttagggtggttgtaagttttattttttgaatttgttattagaagttaaaagaaattttgtataattgtttttgatggaaatataaattggttgatgtaaatgaaatatataaagtagttggtgttttatttatttttataattataaatgaaattaaatgattaaaaattatagattttggggattttttttttattgaggagtttatggaatttgttttttttagtattaataattgtgttgatttatttttttttgtttaattttgtatatattaaaattaggtggttacgaataaaatttagaaatataatttatattttaataaaatgattttaaaattattttattttttattgtggttttatttgttgttttataatgtaggtttttttgggtttttgtttagaatgattttgttaatgtagatgatagttagagttgaatggggaatttagaaattggggattcgggtttttgatgtaatttatatgttaatttattttattagttttttttttatttatagtttggtaaagaatatgggtggagttgttttgggtttatttgtatatatgtttaaattgttttgaaaaaggaagggtaagaaagagtggtatttaagttggaattaggtaggtattttagattaagagacgaattggaaagggaatatttgttagatattttgggtttgaaggtagtttgtgtaagtttttatatttttgagtgtgtgtatatagtggagagggtggagtttgttatttttaaatttgaaaagattgagagattttagagggtttagatgtgttaaaggttagagggattaatatataggttttattacggaaaggcggggaaaaggttcgaatagaaaattgttgtagaagggaagttattgagaggtaagggagtttttgaataattaaaaagttaagaataagtaaaaggaaggaggtcgggtgggggataaaaaaaagtagttgatgtggtaattaagaatttggtgggagtttgggtaggttatttttttttttagattagagttttattagaaatttttttaagtgtttttttgcgtcgttaaagatgataatagtaaattaataagtgtttgaaatgaaaggggatgttgattagtttttaggttatagatttttcgtcgttagttttttttgaatttttatagcgtgtttttgtatcgttttttttaagaagagttattttttatttttatttttaggacgaaggtaagtgtttagttagtatatttattaaatgttagttttggttttagtttttcgtttgcgcggaaagtttattgttatagcgcgtttagtttgtcggaggggtagatagaaaaagtaagtttggtttggcgatttgcggggttacgtatttttagggttggttcggagttttttagagtttaacgtttttgggttagaattgtaaggtttcggttcgagtaaagggtttgagttatcgtagtcgtgggagcgttttttttatttgaatgtatttatttataaataagtataaaatttttttaatagtagaggagaaagatttttgttttaaaattaaagttgggaattatcggaaatttcgttttgagtgcgagatattggtttgttatttttttaatttttatattttttttgtaatatgttttgatttaataatttttttagcgtttagtattgttcggacgttttaattgggtaatttattagtgagttaatataggtatttatatatttttttagtttaagtggttaagtattaatttcgaaatgattataaaatatcggaatcggtaacgtatagtaattttaatttatatgtaaattaattggtttattttaaatgttttttttaaaaaaataattattgtattgtagtatcggaggtatggattaaatttttagaatagataattcggaaataagatttggattaggaagataatttagaatagttaataaattaataatgtttgaggtagttaaatatcgttagttattggtatttatatattcgtttgttgttagataaggagttggggaaattgtttgttagggttgagattataatttagagtgaagaaagtaaatggtagtatatagttcgttatagcgggttttgaaataatattgtattttttttaaattttgatttttgggtgatagggagttggcggaggttattttatatttgtttacggttttgttttaatttgattacgaaattgttgttttttttgagttttttaatttgattattattttaacggttttgttatttgttttaatatattggggggaggagtgtaattgagatttttattaaaaattatttgaatttatttagttagtattgttttatttaagtttagttttatgggttgtatttaattttttgtgttttttatatattaaaattagattattaaaatgtcggtaggaaagggtgaaggaaatggtttaatgttttagtttattggaagattattatttttagatatagtttaaaattttgaggaaataaaaaggatatacgttttggggggaaaatgttttaatattttagaatgggggtattattttttttattttagagaattcgtattggagttgtttatgtaaaaatgtaatatttttgaaatttatagatacgtaaggttagtgtttttttttttttaggtttttagtttaggcgatttagttttaaaggagttagtatttttgatgttataattttgtttatatttgtagggtagagaattgtttgttttgtttggacgttttttttatttttttttaatttgaagtaatcggaatttaaatatagtcgttaaggttcgttttttttttattgttttgataagggaaaaatttgaaatttacgttttaaattagttcggtggtttgtagttttttagtattttgtttttacgattgtatgtttaatgtattttttggtgattttgggtattaattagttgtttaataggagtatgattaaaaatgtaaaagaaggattaggagcgtgaaacgtatgtttagttttttttatatattcgaggagggaatgagaattattttgtattttttatttttttaggagttatttgtattttttattagttgtttattttagttgtattggcgttgggtaaggcgaggatttaaaagtttagcgtagtgtttgcggcggtcgggattggggttaattagtttttggcgggcgagattttagatagaaggggggcgagaggaacgtgagtttttcgagttttttttttttagttttggtttgtaaatttttgaaatttgaaaggggagggagttgtacgcgcgtatttttgcgtttttttagcgtaatttttttttttttttttgtgttttttcgcggatttttgaatttttttgtttttggtttttttatttttttttaatttttttatgagattgtttattttcgttattagttgaaggtaaggtcgttttgttacgagcgttttttaatttttataaaatgaaaagaaaaaaagggaggattattagtttattatttagaggaatggggaggttgtaaaaatcgtcgatgggtagaggtgaagatgtttttttcggattgtatttttcggtgttttgtaattagagtttagttgtgggatttgttgaagaaatttgatttttttgtttcggcgagattttaaaaattagaaatagaaatttttagagttagagaggaaatataattaaatagtacgtgggtatttttttttttatttttttttttttaaataatattgttttgagtttttattgggtaaagagagaaagtttgagtttttacggatgttacgtggaggttagaaatggtttaaaatgtagatttttaattagtttttttcgtggttgaagaggttaattttttttataaaatgagtttatttgtcgattgttagttattttaaagtgaagggatttagtatttaaaataaattgagtaagtttgtttgtttgtttttattgttaatttaaatgaatttaaaatacggagtaatttaagaaaatatataatatgttttagatagtttttaaaagtagggaaagtttagtatttatatagtgattagggttagttttaagcgttaagtttttttaaacgtatttattttatgtatattttttcgagttattatatatttttaaaattgcgagtattggtatattgatttaggaagagtaatataatttttagagggaattttatttttaattagggattaaagagatgtttttttaatagcgggtttgagttttgtttttaagtaggaattaatattggtgggaaaattcgaatttaggagtaatggttgtgtttcggtattttttaaaaatatatattaataggatgtttttgagattgaaaaaatattgttttatatgtttggtagaagtttttatatttggttttttaggcgaattatatttatagtttttttatttagaggtaggatagagttaaaatattttgtttattattaaaatatatatttttgtttaagttaagaaattagaaaattagggtttagaagtaaggtatatttttcgagtgagaatatgttttgtaattttatatattttttgttttgtaggagtaaatgtggatttgagggaaatttttttttttatttttatttttatttcgtgtaatttaatattattttcgttaggaattttaatttcgttattttaaaaaatgagatattcgtgatttagggtgaatttgttgaatgtaggtatagtagaggaaattttagattttatgagcgtttgagttttgtttagtgtaaatttttcgtgaatattgggttagtgcgtggtcgtgtttatttgtgcgtcgatatttttagtatgtttggtttattcgttttgatttcgggcgcggtgttttagttaagttgggtttagcgtttcggttttttttagttgataagtttagttcgttcgttttcggttgtggtttttttatttttttttattagtttattttatttttttagatttttttttatttatttttttttatttttatcgcgtttatttttattttcgttttttatcggttttttattttttttttttcgtagtttttttttgttgtgatttttttttttaattttgtaggtttgaaagaaggttatatacgtacgtttatatttatattttatacgtttcgttttaaataattttatgaatattgttttttgtttcgttttttgggttattttttttgtcgtttttttttagttcgttttgatttgttttttaaaagtacgtttttgttttttcgttgttttggcgttttttttttgatttattagggttgtcgggttggcgtagattgttttttttttttttttattttatttttttttttggtttttttttttatagtgggagttcgtgtttttgtttttcggttggtttttaagtgtttcgttaggtttttttttttttcgttttttcggtttcggttttcgatttttcggttcgttggtatttgttttttttttttgtttcgtttttcgtcgtttttgttcgtttttttcggcgttcgttcgggcgttgtgttcgtttttggatcgttagtcgcgtagtcgggttcggtcggtcgttcgcgcgttattgtgtagtggagtttggtggaatttttgttgacgttacgttattttttatacggagtaggagtagagggaagagagagggatgagagggagggagaggagagagagtgcgagatcgagcgagaaagttggagaggagtagaaagaaattgttagtggcggttagatttcggaggttttagtgtattcgtggattttttcggaatttggtatttttaggagttttgtagtttttttaggttcggttttcgggcgtttgtcgtgtagtcggaggttcggttcgttggaaatcgtttcgggaagtagtgggacgcggagatagtagttttttttcggtagtcggtaagtggaggttatttatttcgtagggatgtgagataatgcgagtttggaaatttgttttatttcggagaatttttatcgtaggtgatttgtggtttttggggttaagtttcgtttaaggtaacgtagtcggtaaatagattttgtaaagttttgtttttttcgtttttcgttatagatattaataatttatagggtgttgaagtcgagagggaagttagatcgtggttggtatttaaaacgaggtatttttttttaaatttcggtgttaatattgtaggaataaattttcgggttaaggattagtatttttaagataaagggttgggtataaagttttagttattggaagattagttttttttttattgttatttattgggaaaaaaaagaaaagaaaaagattttattttaattggtagttagtgattttttaggtttaagcgaattatttgggagttaggtttggatgttaagtttttattatttttttggattgtaatttttttaaattgattattagttaattttaatttggtattttaggagatatattttaaatggatgtagagaattattttttagttggagattaagaaaaaaattttcgattttaaattttcgaaatatgttttttttttttagtttaattattttatttttttaagtaatttagaaattaaattattataaggtggtgtgatttttttttatttttttgtgtgagtattgttttattaaattaaacggaaaaaatttttattattataaatgtaaatattagaatttatatattttaaaatatttttatgaaaaattaatttgatttaaagaaatttttttgtatttgttttagtttattaattaaaattaaagatgtttttattatataaaatattattttggtagaaatttatttaaaatttaaatattaataatattaagaaaataaagtatataagtaaaataaattgaagatttttgttgatgtaatatgagtatataatattttaataattaaattttttttaaaaaattaaatagttattttatttgtggaatgttttattttaatttagtaaaattatatttaaattatttaggtgttttgttttttaagttaagcgtgtttgtttttaaatgtttttaaagtatttatattaattggttgtaaagaacgtatatatatggtaaaatatagaattgaattgagtagtattttaatttttttaaataattatttattataaattaatttattggttaattttataatttagtttatttaaaatatatgtttttgtgttgtttatttttaaattttttattaaagattttgttatggggtaataaagtgtatgaaaaggggggaaatgtgaaaggatttgggattattcgaattgtattttttttgtatttttagttttgcggtagttattagaaattattttttagtaaattgttttattttttagggtttgtttgtttgttttgttatggtttttcgtttttcgttagtcgtgtagtgttttttgtgtgtttataatataaaatttaagttggttaaaataagagtttttggtatatatattttaattagaatatgaattttgggggtgagaattattttttattaggaaaagttttttattttaatttgtgagattagttattgaagttagtttcgaagtttggtagttaaattttttatagaagatttgttttgatagggtaagtttaaggattagtaggcgggaattggaggtttttttttaaaaaattattttttttagttatttagatttagtttttttagtaggtttggttattaaatgaagtataaaaatgtaagttttaaggtttattttgattgtaaaataaatttttaagttataaggatatgtaggagtgagttaaggaatacgttttgattttttttttagtttttagagtggagttttatgagtttttgaagatttgttttgtattgttttgtttggtttttagtattgaagtacggggaagtggggggaagaatgtgtaataattgattgattttatattaagtaacgtaattttttttttttgtatattttattttttaaaaaaaataaataaataaaaattatttgtagttattatttgtagtgtttcggttattagttaataatgtagttagtttagatatataaaaaaaaaagattatcgaaatgatgatgatatgtaaatttttttcgaaattattataagtaaatatttgaagtttggattaataaaattttatttgtgttattttatatcgagttagtagaaagttgtgataatgaattttgtaatattttacgaatagatattttaattagggattaattttgtgattttattgtagaattattaaatttggagtcgttaaattgttatttttgggtttacgggtttataaggatcgaatcggtagagttttcgttcgcgttttcgttagcgggtgggggaatcgtttggtcgtttttattttggatttttacgttatagcgtcgggtagttttttttgtaggtagcgattttggttagaggttttttagggtttagttttttttaggagaggtcgagacgtagggaaacggtatttaggttagaggtaggttcgtagttttttgtttcgtttttgtgttttcgttaattcgataacgtttgtttttatttcgattttcgtattcgcgcgaagtgggtttttcggtcgtcggcgtattttggttagcgtggagagaggtaggcgttgagatcgaaggggtttagggagttttggattttttttttttgtttttaaagtaatcgcggtttttttatttattcggtggagtttttcgagatttatttttttcggtttgtttgtggtagagaagggggagcgcgttaaatgtttggttcgttgcgttgtggttgaaaacgtgaaaaagatttggttcgttcgggagagaaagggggagaattgggtagtagttatattagagttattttttcgtttttggcgggtagtaaattttttaagaacgtttgttttgttttttttagtttcgtttagtttatttagtgttttttttttgcgattttaaattatattttagggtaattatttgtagtaagtaaataaatggtcgggttagtatttttaggagaaagtgtggttaaatatggaaaagtggtttttgatggatgagaggttcgaatttagttcgtttttgaaatattttaggttaagagttcgttcgttttagaattatagaaaatcgagggaaattgttgtttaggataggggtacgttggcgttgatgttttataaatgtttatcgagttttaattaatggataagtattgaagggtggtttttgtatatagttttttaaagagaaaagttttttttatttatttattttcgttgttattgcgtttagatgagtttttaatttcggtatcgagatttttgaaagtaggtttatagtttttttagtatattgtggttttatagttttttaatttttgggtatttttgcggtaattttggagggagatttttttttgataaataaatgttttgggttcgaggttaggttggagatgttgttgtatagttagaggttgttaggtcggaaaaatacgtttgaagtttagtatatagtaggcgtttaatagttagtgtaacgtagttttatttgagttttgtttattcgacggtcgtcgttttttatagtttttttttttttttgttttgtagataacggggaatggagattaattgtcgtaaattggtgtcggcgtgtgtgtaattaggtaagaattttttttttttgttcgggttatcggacgggaggtcgcgttacgtgagggcggtaagagggtattggttttgcggcgaggttttagcgaggggcgttttttcgaggggttagtttgggtaggaaggaaattagaattaaatcgttagtggtttttttttgtggcggggcggtggattaggaagtagcggcgcgttgtgtatcgaagttttttagtttatttttttcggttggaatcgtcggtaatcggggaggcgtagaaagagtacgttattttgtttcgggttgttagagggttcgggggacggggatgtcgttagtttttttttttaattgggtttttgttttttgtttttttttttttttcggtttgtttcgttttttttttttttttcgttttcggtttttttcggtcgcggttcgggacgttttttttcgtacgtggggcgggcgcgcgcgtggtttaggcgtgtagtcggcggtcgttgaatgttttttttttaaagatttcgaaattaaaaaggtcgagtttacggatttttttgagagtcgaaaagaggtagttagtagtaagtttttttcgcggtagtattttggcgttaatggtaaggtcgggagggaagcgtaggtcgcgcgttgggtattcgttttcgggattttgggtttcgggcgaagcgtaagaaggcgaggtcgttagatttgacgcgtttgttgtttgaatttagatatttcgtttttgggtgggacgggaagtagtcgttttagggacgttaatttttttttttaaattatattgtatttttgagatttaatatttttttttttttttcgttcgttttttgtggtttgattttttgcgtacgttttagtaaatttcggcgtttaggttggcgtggaaaagtggtttaatagcgatttttgtcgttcgttatatttcgttgcgcgtagtattattagggtttatttagttatttaggtttttagttacgttttatttagatttgtgggtgcgcggtttatcgcgggaggtaagtaagggaaatttgagttggcgaaggtttgttttggttggttgggggaggggcggggggtcgataatatttttgaagagttggagggtagttattgtgtttagtagtttagggtagaatggaggtttgtttttgtcgacgcgaatttgtttgaagtattggttgttaggttttgggttttggcgatgtcgttgtttgattggttggttttattttggaggaatcgagggatattgttagaggaggtttataggtttatgtaaaaagttaaaaagtttttaatttacgttataggttttttttgaattgaaatttgttttatggggcggaggggggggtgtaagggatggaggagggaagatgtttttttttttaaatatatggaaaaaaattttttaaatttattgtttttttatttttttggtttcgtagtaaataagtgtttagttttaggaggttattgatttttgataatgtgagtagataaagtttttttttttttatagttttcggtttttaatttttttttttcggattaaagtgtaagaatataaatgtaatatgggatggaggggggcgatttgggatttcggttaaaaaaataaatcgtattattaagaagaaaataaaggttttgtattggagttttttcgtgaatttgagagaaaatgattatttgttgaaatgaagcgtttaaagcgatttagtgttttacgttcggatattgtattatatcgttagtcgttttgttgggttagttaaacgtttatttgttcgggattaattttatggggttaaatgggggtaatgtagagataacgtcgcgtgatttttgttatttagattgtgttaaattttttttttgtttgataatcggtagtaaaaataaattattagatcgtagtatgtttgggatatggttaaaaattaagagtagcgatgatttttggggagaatgttttgcgcgggtttagttttggtttcggttagattagaggagttttttaatttcgtttcgcgcggggcgggttcgtagtcgttaagtcgaggttgatattttttattgtgttgggagttagagagacgtaaaatgttttttttttttagtttttattttaggttttttagatatggggaatgtattttgaggataggtggagaagtttacggtaggatggggttttcgtaggtgagtaggaaacggttaagagtagaggagttttgtttgcgttagttataagtcgcgtaggcgtttttggtcgttcgtttttgataattagtatataaagaattagaaataatgaatgattgtttttttaattattatttttaggttcgtattgttttagtgtacgtgaaaggttttttttttatatttaatatgtttttttttattttttgatcgaaaagaaaaattgttgtttaaatacgtttaatgttattaattaagaaaaggtatgtaatgggaagaaatgttgaaaattttgatttaattggtttttaaggaattagtagacgataaaaaaaaattatacgagtgggtaaagttatagtattgttgaaggatagagtatttatttttttttgattttaagttaatttatggaatatttaaagttttggttatagtttgtttgtaaaataaaaggatttattttttgtgtttttttaaagtttttttttgtttttaaagagaaaaaaagtttataatgatatatgatttttttaaaaggtcgtgatagtttattacgttatttttttcgtttttgtttttaacgtcgtttaaaaatattatgttttcgttaaagattaaacgttttgtataggtagagtttatttttaagtagtttaggttttgttttttttttttagtgagttttattttttttggtatttattgggcggatgtttagtttggatagaatttcgaaacgggggtagtacgagagcgattggagatttttaaaagttagaggtttgagagagggtggacgtagttagtagaagatggtgtagaagttagttgagaacgattttttagagtaaagagatttttttttggttttttttgttttgggggttttgaaaggaatttataaaatggtttttatttttaggaggaggacggattgatttttttttgttattggtttaaaaagttttagggcggcggttttgggtttcgtgttgaaatcggattgtattgtagtttttttggatttgacgtttggttttgcgtttcgataaggggcgggtatttttttcggttttttttaggaacgtattaattgttaaatagttttggtttagtggatgggttgaaagtgttcgatttaagtcgttggtgtgtatagattttttttttttgggaggtgggttttatggttcgttgtggtatttttagtcgcgatatatatttttatacgcggtagtagttcggttttaatttttttcgaaggatttgggttaattttggcggttttggcggtcgtagattttttttcgtcgtttcgttttcgcgttttttatttaattagcgaatgtttgcggagtatatattacgtggatttttaatgtattttttgaaagtaaataatatagtttttttcgtcgttatgaagggattttaattttaatatggatattagcgagattagtttagatcgtttttagtaaaacgtaaaatggtggtgcgtggggtggtgattaaggttttgagttttgttagaaagaaggggatgtgtagagaaaggtggagaattttagttgtggttagcgcggaagggataggtgtttgtcgaagggggtatgaggtttgaggaaaaagtaacgaaataggggtaaggagagttttttattttttttttttcgttcgattttcgttattttatttttttttttttttttttatttttcgcgttaattaaatttgtagtttatttgaaaggtgtttcgtcgcgttgtggttttttatttttaggggaaattgtattagttgttcgaaagtagttagtttttgcggatttttgttcgtaaaagtggtttttataggtcgcgtttttcgttgttgattcggtatataaagttttttaaggttggttcggttgttattttttatcgttcgttgttaatatatgtagtagttgttagagcggttcgggggaaaaggaaatgtataacgaaagtttattcgtgagtaggaatatattaatggaataatttgatgtttttttaattttatgtaaaaagttttgtcgtttttttaatattgattgaatgggtaattaatggttttttatttaggcgaatattttgtaatttaagataggtaaaagataataagtttaaggtagaagataaaaggtttaattgtagcggcgtttgttcgttttttattttttagggtttttgattaggaaagtttttttttagaggagaaaaaggtaggagtgggagaatatatatttattatttcggggttagattttatcgtagtatttgattatttagtttagttttttgttattttgtttttttatttttagtttttttttttgtatttttttttttttttaatttttttaggatgattttttattattatcgttattatggttttaataatttttttttttaaattttatattttttattttagtaattaatgaggttgttttttgatttaggaggagattttttttttttagaatttaatgcgtagagtttttgagaattaaagtagttggtaggggaggaagaaattaatagaaagggagagagtatatagaattgtgtgtgtatgttaaagagcgattaggaatgatagagttaatttttttgtgaggatttgacgggaagagtgtttaagattttattagtatgtttttaataggttgatattttaatttaaatttttagaagtaatatattatttgggttattataatgaggtgggtttttttttttttgttagttgatagtttttaaaatattatttcgttagggaaataaaagttttattttagattataggtgggtatttttggatttaggtgatttatggttattatgataattaatgttgaatgttagttattagtatgtttgggagagagaaaatagaaagaagggagagtaaaagaaatagaaaagggagatggatataagttggagagggaagaaaagagaaaaagaggaagatagatgagtgtttaatttaattgttgtttaaaaaagtggcgggggggtgggattttatttagttttttgttattttttttttttttgatttggatatttatgtttaattttatattttatttttttttttttttttttaaatatatgtgttatattattttttttattttatttagttcggtaagtagttgttttttggagatttagtgatatttaggaaaattgtggtagtaatatgtaaatgtgaggaagtattaatagtatgttcgttgagtgattttagtaaatgtttttttttttaatttttttttttttttttttttaggttattgtgaggtggtaatttttattgttatttgaatattgtttttttaggtagttattttaaattttaaatggttgagtagttagagttgtgggttggaaaaatgggtattatttgtagggatttagagagggtggttgttgtttaatatatttatagatttttaatttagaaaataattttttttttttgataagttagagttttttaaattttatttaggaaatggggaaaaggatagttatagtgaagtttttaatttttgggttatttggttttatagttatgaggggtggtgggtagtggattgtttttagtttggtttgtatgtagagaaaagttagatattggagggggtggggtatttttcgggtaggatgtaaggtttttatttgatttttgcgttttattaggagtttatatatttacgtttattacgtggttttaagttgagtttaggcgggtttcgtttttgagttagtttgggtagggtaggatttttattcgtttaaggtttaatagtttagggagatgtttaattaagttattttttgggtgaattttgaagatagattttttttaaaagttagagattatttggttgagttttaggttagattgatacggagagtttggcggtatagtttaatcgtttattgttatggttagagggattttgtataattaatattgaagagtgtgaaattaaataagattttaagattggtaatcggtggtaaatattagtataaaatacggttgattttatgttatatattttttttttttagggtttttttttgaaagaataagtaagaaattttaatcgagataatttttgatgttttttagatttaaaattttacgtcggtattgggttttttttttttgttttacgtgagttatgtagtatttttagtttttttattaggattttattaatgtttttcgtattggaaatttttgtgttagaggttgaatttatagtaatttttaaaattaattaagaagaatttagttagaggttatagtaatgttggaattataaaatgtataagatttatttttttttggttttttttttatttatgttgtttatgtttgtgtatttataagttttatgtatattaaatttttaaaattaattattattatgttatagagtttttattggatagtgttttttagtttttattatatatttttttttttttatgtagatttattatgttggtgttttgttatatagggggtttgagaagaatgttatttaatcgtcgttgttgtgagtgtgtaaagtgattaggagattaggagaatgttgaaatttttgttggaaaaatgtaaagaaaatttttattttgagttagttgtttatagagttagtgtgtgtgtgcgtgtgtgtgtttgtaatataaaatggatgtgaatatatatatataaatagatatggttttgtttttattttaatttgaattatttagataattgtttttatttattatttgattttaatgggtttatataaattaggatattttatttttttaggtatttaggttgttgttgatttttagtgtttttaatatttcgtatacgttggtattatgaggagtagttacgtgtttttgggttttttaattattttggaggttgattgaggttttttatatatgtatatttgtcgtgatgaaagttttatcggtagagtggagttattagagtttttattaaaattttgtgggtttatgagagatgggtttagaaatttatatggttttgtggggtttttcggttttttaaaataaggtattaatatttaagtttttaaaaatatttgtagttttggggtttgaattttgaaaaataaggagtgaggggttgtgtatattaattatagtggagattttttttattttttaatgtgatggagtttttttatgaaatgaagttttaaggggtatggtattgtggggattatagttattttgaggtttaaaagaagaaattggaatatgattagtaaatatatttagtagaaaagagttggatttttattgatttagttataggttatcggttggtagtgtaatgggaggaaatatttattttatatatatattttatgattttgggggaattagaggaaatttaataagaaaacggttagaaatatttaaaatttttatttaaaagatttaagtaaattagagttttattagattaaaaattattataaatgtaagagtattgtttttagtgaaacgttgtggggtttgagaaggagatttttcgttaaattttcgggataaaatgcgttatttaagtattagataatgagtagaatgtaaattaatttaattttttttattaataggttgttagtgtaatgtgtataatttagtgataagattgtaggatttaatatagttggatgtatgagttttagttaatgtagatttgttatatgaggatgtgttttattttgagtaggtgtttgtatgtgtggaatggggtaaagtggaataaaaggttaaaagtagaaatgttgatttaaagtttattatgaagaaattttttttttgtagttaaattatttttaaagtgggatgatattggtgaagaaagattgaaaaataatttttatgtgcgtttttggattgtaagtttaaaatggggaggagttgtagatagggtttgggggtggttagggtaaaggagagatatataagttgtaaatatatttgtagtttgttttatttattttgttttatatcgaataagttttttaattttgtgaataaggataaggagggagtgttttaaagatattttatgttggtattgtaaattattgattgtaatgttaaataaatatatatttagagatgataatattaattttatagtaaaataatcgtttatgtagaaatttagaggagattagtttgttttttttagttgatttatgttgggggataaaaggatttttaaaaattattttgaatatgtttggatttttttttttaatttttttggaaattaaatttgtttggaaatagtgttataaagagttgatgtttttaaaggtgattttttttgttttatataaataaggttttgtttttgttagttgagcgtagttttaggtttttcgtttttagtttatatatattttttttgtttgtttggattttaatggtttaagatagttttgagtttattgggaaaagaaaatgattgttaaaaattatttttgaaattggttatttggtaatatttttaattgtatggaaatttattaaggtatattttatatataattagtttaaggttgttgattttataggttttatggatttaaatttgattgataataaagtaaataagagagtcgaatttaaagcgtggttttttcgggttaggacgagtttaatatagtgtataaggaatttgaaagatttaggatatgtgttttaattaacgttaagtagaatggataagtttttagtattttgaaaacgttgggttagggttttttttttattgtgtgttttttgtttggggattaataagtattatagagaacgtgatttgaggcgattttttatttttgtataaatttagagtgaattattaaatagttgttcgtttaaagttaaggtaatttttttttgacgggtttatttgtttttcgatttttaatttattagtttgtttttttagggttttgttttttttgtaattaaagtttttttagattagcgtagtatttatttgataggttgtttggaaaatttaagatcggagaggtgatttgttgttgttttttaaattttttagttttaagtaacgtgttttttttttatatggggtgggggattggaaatggatgtagtgagatataaagagtgggtgttttgttgatttttgtatttttttttttttgattattttattttttttttttaagttttcgatttttagttttatttttttatttttgggttcgtattaaaagtcggatcgttttgggttgggtaggagttgaattttcgggagtttgtttgtgtagatttagtgcgtacggcgaggtagtagttcggtttcgtattgttgataggtgtaggtaggatagtttttttatcgcggttcggggcgttttgattggtgcggagttacgttagtcgtattcggagaagggtttgggaggaggcggaggcggagagggttggggagggtcgcggcggagtgacgtttcggtattaggaagttcgtttttggttttaagatgttaggttaatagggaagcgcggagtcgtagatttggttcgtcgttcgtttgggtgtttggagttgagttgcggtaaggttcggtttttgttcgatcgttcgaggggtgtgcgtgtgcgcgttgcggagggtgcgtttagagggtcgcgtcgtggttgtagcggttgttgtcgtcgtaggggatttaatattatttatttgtttttgttatttttgatatttttttgttagggttgtcgcgtggggggggggcgggtagagcgcggtcggcgttagttttttttattggaggggtttttgggggagggagggagagaagaagggggtttttgtttatttttgtttcgttttggagtttggaagtttgttttttaaagacgttttgagtggtgtttttttgtttatattttatgttttcgtttgttcgttgatttttcgttttcggattttttcgtttgagtttttcggaggagacgggggtagtttggtttgagaattcggcgggggttgcgttttttggtttttttcgtagcggggaaatttcgcgtttagagcgcgattcggagcgggtagcggcggttacgggggttcggcggggtagtagttaaggattagtagagcgtcgcgttttttcgtttatgaattgtatgaaaggttcgttttatttggagtatcgagtagcggggattaagttgtcggtcgtttttttatttttttgttattatttttagtcgttagttatggtttcggttttggttttcggttagtttcggtcgttggatttttttaagtataggttggaggtgtatattattttcgatatttttagttcggaggtcgtaggtaaggcgtcgcgtcgttttgtagatattttcgtttagttgttttgcgttattcgttttttttcgttttaaggaagttagttttttcggggggaggcgtggtgggagtggtcgttcgtttggtttttcgtagaattttcgggagtcggaattttgattatttcgtatttttttagttttttttcgatcggttcggtttttggggcgttaagggcgcgagtaattttgtcgttttttttattcgtattttggttttttttttgttttttgggttataaaaattttagtattttgattcgaggatttttagaggtcgtcgatttttgtttttgtttttttttcggtttttagttttcgaggagttttattcgttaggaaattgtttgaaattatttagaaatgtttttcgcgaagaggtattttttttttttttttgggaaagggtcggcgaatttcggtgtttaatcgaatttttatatttttttttagtttttttaaatcgtatggaaatttgagttttttgcgagggggaggggggtttgtaaattacgcgcgtgtgcgcgttttaggagatttggtgtgtttgcgtagaggtgtataaatatatttgaaagtataggttataaaagtgaatgtgtcgttgtagtgagataaatatgtaaataaaacgtgcggcgttgggggaggggaggaaatggggcgcggatatttatatttgcgtttgtatattttataggcgtagcgtttttcgcggttcggagtcgtcgcgcgtattttttttcggcgttaggtagtttagtttttttacggtttttgtcgtcggtttagttggcgttcgcgttgtaggtgggtatgttgacgggaaagtgtgtgtgtttcgtttttagagaaagataaaagttagtaggggaagaatgaggacgtgggcgtcgaggattcgtttaagaagaagcggtaaaggcggtagcggatttattttattagttagtagttttaggagttggaggttattttttagaggaatcgttattcggatatgtttatacgcgaagaaatcgttgtgtggattaattttacggaagttcgagttcgggtaggagttagtacggagtttgggagggatggggggaggatgttgtggaggtataggttaagtagattaggagagaatgtggaaggtagcgtcgtttgggagggcgtcggtggggcgtagttttgtaaaggtagaaggtttcgcggcggtttggttgcgagattatagtttttttttcgaggtcgataggattgtcgttttggtttaggtttttagagcggtatcggtttattgtttcgttatttcgcgattttacgagttgggttgtatgggtaattttttgtataggatattgtgtttttggtttgtagttgttagagtagagttaataaaatttttattaggttaagagtcgcgaataggttttaatttgtgagtttttaataaggaaaattcgttagagatacggaagagttggttttttttgggaaatttttgtttcggttttggtttagtttttttttttttgggttcgcgttttttatattttttttacggttgtttcggttatttaggttttttttatatattttattttttagttttgtgattttcgggagtaaagttttaatatataattattagtttttttagaaggagaaagaaaaaaagaagaaagatttttttgtttggtttatttatttttttttaggagttgaattttggaaattgaaatttatatttttttttttaaattataattatagttttgtaaaaagggtttattttaattttgtagtaaatttgtattttatggattggtaaaaatgagtttaaataaataatttaatagtaacgttttggtttatgttggtcggtggaagattttaaatttgttaggattttggaagtagaaaatagaattaagtaaattaagcggtatttagaggttttgttgttaaaaaaaaaaaattaagtgttttgggtagaaaaaataaagttttcggttagagtagagtaaataaaaagaagaaaataacgataaaaagaataaagattaaaatgtttttttaaattagagggaatgaagatattttttgggtggtatttgtgtaaggtatgaggttatgttggtggataaaaggtcgggaagaagttgaaaatggttttagtttaattgtttagagttagagttgggttttgggcggcgtggttttgagtaaggttagttttttattagtttttttgtatattaagggaacgggttttttacgtatttttttcgtttgagtaaagtttagatggtttagggtagaaatggtaagtaattaaagatagagtttatgggttttttgggatttttcgaaaacgtttttttatttcgttcgttatttcgtagttttattttagtgttttgtagtcgcggcgttgggttttttttgtagttgtttttttttttagggcggttgtttgtcgagttaagtgggagtgaggcgtgttttttatagtagtcgggtgtaaagaggaagggggataaaaaggaaattaagaatgaaaggaaaaagagaaaaagcggattatacggttgggttcggcggagatgtgtaatgtgaaatattattggtgttagttcggatattttaggttaggtttttttttaatatataaaagtcgtcgtttggggcgatagggaggttcgatgtggattgggatcggggttgcggttgggttatcggatacgggtggaagtcggtcggtttgggtggtcgtttgtaaagttaaacgattcggttgggtttggcgcgcggataggtttgtggtgggtttagggtaaagaagaggtagagcgaaagaagggggaatttttaaaattatttttttcgggttttcggagtttaatatgttaagtttttggagttaacgagttgacgaagaggtggttttttgttttttatttggttgttttgttaggcgagaaagagtgttggcggtttagtttttgttaagggagtacgtattagggggtgggggacgatagtggaggttagggaaggaagggaggaattgcgtgggagaaagagcgattttttagtgtttttttagttttttttttttattcgtgggtttgtggttttggaatggaagtaagtttgtaaggtgtttcgggaagggttggaaaagtttgttgtttcgcgtttgttttatattaagtgtttttggatttggagaaacgtttggttgagtgattaaatcgttcgtaggtttttatgcgttcggttgaggtttgtggcgtagtttcgagttttagttcgtaggttagagtagattaggttttttgcgtttggtggagattcgggttagtaattgaaagttggttttggtattttggtgtgtagggcggtgtagtgaagcgaggttagggtgtgtgagtgcgttagcgtgtgtgtcgggggaaggcgggggttggttttcgatggaagttttagtaatttgtattgtggtatttgtttgtttttttgttttaatcgtttttaggtttggtttaagaatcgtcgggttaaatggagaaagagggagcgtaattagtaggtcgagttatgtaagaatggtttcgggtcgtagtttaatgggtttatgtagttttacgacgatatgtatttaggttatttttataataattgggtcgttaagggttttatattcgtttttttatttattaagagtttttttttttttaattttatgaacgttaattttttgttattatagagtatgttttttttatttaattttatttcgtttatgagtatgtcgtttagtatggtgtttttagtagtgataggcgtttcgggttttagttttaatagtttgaataatttgaataatttgagtagttcgtcgttgaatttcgcggtgtcgacgtttgtttgtttttacgcgtcgtcgattttttcgtatgtttatagggatacgtgtaattcgagtttggttagtttgagattgaaagtaaagtagtattttagtttcggttacgttagcgtgtagaattcggtttttaatttgagtgtttgttagtatgtagtggatcggttcgtgtgagtcgtatttatagcgtcgggattttaggattttgtcggatggggtaatttcgtttttgaaagattgggaattatgttagaaggtcgtgggtattaaagaaagggagagaaagagaagttatatagagaaaaggaaattattgaattaaagagagagtttttttgattttaaagggatgtttttagtgtttgatattttttattataagtatttttaatagttgtaaggatatatatataaataaatgtttgattggatatgatattttaatattattataagtttgttattttttaagtttagtattgttaatatttaaatgattgaaaggatgtatatatatcgaaatgttaaattaattttataaaagtagttgttagtaatattataatagtgtttttaaaggttaggttttaaaataaagtatgttatatagaagcgattaggatttttcgtttgcgagtaagggagtgtatatattaaatgttatattgtatgtttttaatatattattattattataaaaaatgtgtgaatattagttttagaatagtttttttggtggatgtaatgatgtttttgaaattgttatgtataatttattttgtgtataatatttcgtataatattattgttttattttttagtaaatatgaaataaatgtgttttattttatgggagtaaaatatattgtatataaattggtttggattttttttttttttttttgttattaatttggttaggatattttagttattgttttttaaataaattagttttttttgtttgtttagttaaatatataaggtagtagtttttatttaaatttggtagaaataaatgatagttatttattagaaattaaaaagaaaaaaaaaggtattttcgggggggaaaagggttataaaatttaattttgtttttttaatttttttttggtttaaatttagaggattttattatggttagtaaataatatgaaaaagaaaaaagaagaaagaaatttagtaagtttattagtttaaaatgatttttaagtttatttttttacggggaaatttatatttttagtaaattgttttggagaaatatttgtgtatgtatatatgtatagtttatatgtattttttttaggaggaatatatttataataaatttatagggaaatatttttagtttaaaatatttaggtttttacgtttatttttaggtttaagtagagagattttttatgttatattgtattattatttttaaattttttggagatattaaaagaaataaagatgatttttaataattatagttttttagttttttaaagaatttaggggttgagaggttagagtggagttttttgagttttgtcgagtaatatgtagttgaggtaaaggttatgttttcggtgttttgttttaaataatattgatttattaattttaaatttgtttgtttttgaaattatataggattatagtttgtaaattgtaggataatgaagtaaattaagatgaattatagttttggttttttttgttattttttgatatttaaatagggaatgagttcggtgtgagtgtttaaatgaattttaagtattcgatttttttttattcgcgatttttagttttaaaaaaatgtgaaatttgattttataataaatagaaataaatattatttagttttagagaatttatttttatggcgttaggagggtcgttgtggaggtggggggagggatgtgttgagattttttgttatgtttgttaatttttcgtataattaaagtgggcgagaataaatattacgttggggaatttagagtaaaaagtaatcgtcgattttttggagtcgataatattattgttttttcgttttagttgtttttatttgaatgaaattttatttagaagtttttggagttcgaatattgagtttttgttttgtaagaaattgttgttgttatttaaagagatcgtagataatgttttcgatttaagattagtgtttaacgtatattttttttttttttaaagttttgttttcgatttgcggagggattacgtagtagtggggggtagataaaagttttttggacgagggttattttttattatgttgtttatttgagagtagtggagaggaaaacggtttttacgggcggattttggtttttggagtcgtagggtttagcggtttcggttttttcgttttttagtttggtagggggcggggagggttaaagggcggaggggaaggaaggagttagaagaggggatttggggatgggggttggaagcgttaacgagatttgttcggaggatttaggttttttgtaggttggtgagtgatttgggagttttgggtaaaagaggtgtatttcggtttagtttggttgttgaattagaataacgcgaggatattaattaatttgtaggaaataaaaaatggaagtcgaggtttaggaagagttgttattttttcgatttgagtggtgtaggttgggggcggagatttgggatttaagagaggttattttttttattttagttttttttttttggatttttaaaaggaataattttattttttttttcgttttttttataatttttattttcgttagttcgtaggtcgtttgttttttttcgtattttttttttttatttagcgagagaaatttagtttttagagt

SEQ ID NO: 4 - DNA, 28536 bp, Artificial Sequence
  chemically treated genomic DNA (Homo sapiens)
gttttgggagttaggttttttttgttggatgggaaaaggaggtgtggaaaggggtaggtggtttgtgggttagtggggatgagagttgtggggagaatggaggagagagtaaaattattttttttggaagtttaagggaagggagttgaggtgagagaaatggttttttttaggttttaagtttttgtttttagtttgtattatttaagttggggaagtgataattttttttaaattttgatttttattttttgttttttgtagattgattgatgtttttgtgttgttttagtttagtaattaaattaggttgaggtatattttttttatttagagtttttaaattatttattaatttgtaggagatttaaattttttgggtaggttttgttagtgtttttagtttttatttttaaatttttttttttagtttttttttttttttttgttttttggttttttttgttttttattaggttgagaggtggagaggttgggattgttgggttttgtggttttagaagttaaagtttgtttgtggggattgtttttttttttattgtttttaagtgaataatatggtgggaggtgatttttgtttaaagggtttttatttgttttttattgttgtgtgatttttttgtaagttggaggtagagttttaaaaagaaagaaagtgtgtgttgagtattgattttaaattggggatattatttgtgatttttttaagtggtagtagtagttttttgtagaataaagatttagtgtttgagttttaaaagtttttaggtaaagttttatttagatgaaagtaattaaggtgaaaaaataatgatattgttggttttagaaaattggtggttattttttgttttaagttttttagtgtggtgtttgtttttgtttattttggttgtgtggggggttgataagtataataaaagattttagtatatttttttttttatttttataatgatttttttagtgttatgaggatgaattttttgggattaagtggtatttgtttttatttgttatgaaattaaattttatatttttttaaagttgaaaattgtggataaagaaggattgggtgtttaaagtttatttaaatatttatattgaatttattttttgtttagatgttagaggatggtagggagggttagggttgtaatttattttgatttgttttattgttttgtagtttgtaaattataattttgtataattttaggaataagtaggtttagaattaatgggttaatattatttaaaataaaatattgggagtatgatttttgttttaattatatattgtttgataagatttaggaaattttattttaattttttaatttttgagttttttgagaaattgaagggttgtagttattagaaattatttttgttttttttaatgtttttaaaggatttggaaatagtaatgtaatataatatgaaggatttttttatttaagtttgaagataaatgtggaagtttaaatattttgaattggagatgtttttttgtaaatttattatagatgtattttttttgagagaaatgtatataaattatatatgtatatatgtataaatatttttttagaataatttgttagaggtgtaggtttttttgtaaaggagtaaatttgagagttattttaagttgatggatttgttaaattttttttttttttttttttttttatattatttgttagttataatggaattttttaggtttaagttaaagaaaaattggagagataaaattagattttgtagttttttttttttttgggaatgtttttttttttttttttagtttttgatgaatggttattatttatttttattaaatttaaataaggattgttgttttgtatgtttaattaggtaggtagagggaattggtttgtttaggaagtagtgattgagatgttttggttaagttagtgatagaggaggggagaaagaatttagattaatttgtatgtagtatattttatttttatgaaataaaatatatttgttttatatttgttgaaaagtaaaataataatattgtatgaaatgttatatatagggtaggttgtatatagtagttttagaaatattattgtatttattagagaaattattttaaaattgatatttatatattttttataataataataatatgttagaaatatatagtgtggtatttagtatatatatttttttgtttgtaagtgaaaaattttaattgtttttgtataatatgttttattttaaagtttaatttttaaaaatattgttgtgatattattaataattgtttttataaaattaatttgatattttgatatatatatatttttttagttatttaaatgttaataatgttaaatttaaaaaataataagtttatagtaatgttaaaatgttatatttagttaaatatttgtttgtgtatgtgtttttgtaattgttagaaatatttgtagtgaaagatgttagatattgaggatatttttttgaaattaaaggagttttttttttgatttagtggtttttttttttttatatagtttttttttttttttttttttttagtgtttatgattttttagtataatttttagttttttaagggtggagttgttttatttggtaaggttttaggattttggtgttgtgggtgtggtttatatgggttggtttattgtatattggtaagtatttaggttggaggttgggttttgtatgttggtgtagttgaagttggagtgttgttttgtttttagttttaggttggttaggtttgagttatatgtgtttttataaatatatggaggagttggtggtgtgtaaggataggtaggtgttggtattgtggaatttagtgatgggttatttaggttgtttaagttatttaggttgttgagattggagtttgggatgtttgttattgttgagggtattatgttggatgatatgtttatggatgagatagagttgggtggggaaaatatgttttgtgatgatagggggttgatgtttatagagttgaagaaggggaagtttttggtggatagggaggtggatgtaaggtttttggtggtttagttgttgtaggaatagtttgggtatatgttgttgtagggttgtatgagtttattgaattgtggtttgaagttatttttgtatagtttggtttgttggttgtgttttttttttttttatttggtttgatgatttttgaattaaatttgggggtggttggggtaagggagtaaatagatgttatagtgtagattattaaaatttttattggaggttaatttttgtttttttttgatatatatgttagtgtatttatatattttggttttgttttattgtattgttttgtatattaagatattagggttagtttttagttattggtttgggtttttattaagtgtaggagatttggtttgttttggtttgtgagttgggatttggagttatgttataaattttagttgaatgtatggagatttgtggatggtttgattatttagttaggtgtttttttaggtttaaaaatatttaatgtaaaataaatgtggggtagtaggtttttttaattttttttggggtattttgtaaatttgtttttattttaaagttatagatttatggatgaggagaaggggttggaagggtattagaggattgtttttttttttatgtaattttttttttttttttttgatttttattgttgttttttattttttggtatgtgtttttttaatagggattaggttgttaatatttttttttgtttagtaaaataattaaataaagagtaaaagattatttttttgttagtttgttaattttaggagtttggtatattaaattttgggaatttggaaagggtagttttggagattttttttttttttgttttgttttttttttattttaagtttattataggtttgtttgtgtgttaggtttagttgggttgtttggttttgtaggtggttatttaggttggttggtttttatttgtgtttggtggtttagttgtaattttgattttaatttatattgggtttttttgttgttttagatggtggtttttgtgtattggagagaggtttggtttgagatatttgagttgatattagtgatgttttatattatatatttttgttgggtttagttgtgtaatttgttttttttttttttttttttatttttgattttttttttatttttttttttttttgtatttgattgttataaaaagtatgttttatttttatttggtttgataagtagttgttttggaaggagaggtagttgtaaggagagtttagtgttgtggttataaagtattagggtggagttgtggaatagtgggtggggtgggagggtgtttttgaaggattttagaaaatttatagattttgtttttaattatttgttatttttattttaggttatttaaattttgtttaggtgagaagagtatgtgagaggtttgtttttttgatgtgtaagagagttaatgaaagattgattttgtttaaaattatgttgtttaggatttagttttggttttggatagttaaattaaaattatttttaatttttttttggttttttatttattagtatagttttatgttttgtataaatgttatttagagagtgtttttattttttttgatttgggagagtattttggtttttattttttttattgttgtttttttttttttgtttgttttgttttaattgggggttttattttttttatttagagtatttaattttttttttttaatagtaaagtttttggatgttgtttgatttgtttgattttgttttttgtttttagaattttaataaatttggaattttttattgattagtataaattaggatgttgttattgggttatttatttgagtttatttttgttaatttataaagtatagatttgttataaagttaaggtaagttttttttataaaattatgattataatttagaagagggggtgtgagttttaatttttagagtttaatttttgagagaagataaataaattaagtagaaaagttttttttttttttttttttttttttttaagaggattagtagttgtgtattaaaattttgtttttggagattataaaattaggaaatagggtgtgtgggagagatttgaatggttgaaataattgtaaagaaggtgtaagaagtgtgagtttaggagggaaaaagttgggttagggttgggataaaggttttttagggagggttaatttttttgtgtttttggtgggttttttttgttaaaggtttataggttggagtttgtttgtggtttttggtttggtagggattttattagttttgttttggtaattgtaagttaggaatataatgttttgtgtaggggattgtttatgtagtttagtttgtgagattgtgggatggtggggtagtgagttggtgttgttttgggagtttgagttagggtggtagttttgttggttttggagagggaattgtaattttgtaattaggttgttgtgaggttttttgtttttgtaaagttgtgttttattggtgtttttttaggtggtgttgttttttatatttttttttggtttatttggtttgtatttttataatattttttttttattttttttagattttgtgttggtttttatttggatttgggtttttgtaaggttggtttatatagtgatttttttgtgtgtggatatgtttgggtagtggtttttttggaaagtggtttttagtttttggagttgttggttggtaaagtgagtttgttgttgtttttgttgtttttttttagatgggtttttggtgtttatgtttttattttttttttgttggtttttatttttttttgaaaatgaaatatatatatttttttgttagtatgtttatttgtaatgtggatgttaattggattggtggtagaagttgtggaagagttgggttgtttggtgttggaggagggtgtgtgtggtggttttgggttgtgaggagtgttgtgtttgtggggtgtgtaggtgtaagtgtgggtgtttgtgttttattttttttttttttttagtgttgtatgttttatttatatgtttattttattgtagtggtatatttatttttatagtttgtgtttttaagtatatttatatatttttgtgtagatatattaaattttttgggatgtgtatatgtgtgtggtttatagattttttttttttttgtagaaagtttagatttttatgtggtttgggaaggttaggaaaagatgtggggatttggttgggtattgaagtttgttggtttttttttaaaaaaaaaaaaaaaatgtttttttgtgaagggtatttttgagtggttttaggtaattttttaatgagtggagttttttgggagttgaaagttgagaggaaaatagggatagaggttggtggtttttgaaggtttttgaattaagatgttgggatttttgtgatttaggaaatagaagggaggttagggtatgaatagagagggtggtagaattgtttgtgtttttagtgttttaggagttgggttggttgagggagaattaaagggatgtggggtagttaaaattttggtttttggaagttttgtggggagttaggtgaatgattatttttattatgtttttttttggaggggttgatttttttggggtgagagggagtgggtggtgtagagtagttgagtgggaatgtttgtagggtggtgtggtgttttatttgtggtttttgggttggaggtgttggagatggtgtgtatttttagtttgtgtttggaggagtttagtgattggggttgattgggagttagaattgaagttatggttaatggttggggatggtgataggaagatgaggagatggttgatagtttggtttttgttgtttggtgttttaagtgaagtgggttttttatgtagtttatggatgagggagtgtgatgttttattagtttttggttattgttttgttgagtttttgtagttgttgttgtttgttttgggttgtgttttaggtgtggagtttttttgttgtggggagagttaggggatgtaatttttgttgagtttttaagttaagttgtttttgttttttttggaaggtttaagtgaaaaagtttggagatggaaagttagtgggtaaatgaagatatgggatgtgggtagaagggtattatttagagtgtttttagggagtaggtttttaagttttaaagtgaaataagagtgggtaaagatttttttttttttttttttttttttttaagaatttttttaataaggaaagttaatgttgattgtgttttgtttgttttttttttatgtggtagttttgatagagaagtgttaagagtgatagggataggtaggtgatattagattttttgtggtggtagtagttgttgtagttatgatgtggttttttgagtgtattttttgtaatgtgtatatgtatattttttgggtggttgaataggagttgggttttgttgtagtttagttttaggtatttaggtgagtgatggattagatttgtggttttgtgtttttttgttggtttaatattttaaaattagaggtgggttttttggtgttgagatgttattttgttgtggttttttttagttttttttgtttttgtttttttttagatttttttttgggtgtgattgatgtggttttgtattaattaggatgttttgagttgtggtggagggattgttttgtttgtatttattagtagtgtggggttgggttattgttttgttgtgtgtattgggtttatataggtaagtttttgggaatttagtttttgtttagtttaaggtgatttggtttttagtatgaatttaaaggtgaagagatgaggttaggagttgaaggtttgggagaagagagtggaatggttaagaagagaaaggtataaggattaataagatatttattttttgtgttttattatatttatttttaattttttattttatataaaaaggagatatgttatttaaaattagaaaatttgaaaaatagtaataaattatttttttgattttaaattttttaaatagtttgttaagtgaatgttgtgttaatttgaagaagttttaattgtaaagaagatagagttttgaaaaggtaggttaataaattagaaattgagaagtaaatggatttgttaaaagaaaattattttgattttaaatgaataattgtttggtggtttattttggatttatataagaataaaaagttgttttagattatgttttttgtgatgtttattagtttttagatagaaaatatataatagaagagaaattttaatttagtgtttttaaaatgttgaaagtttatttattttatttaatgttgattaagatatatattttagattttttaaattttttgtatattgtattaagtttgttttaatttgagagagttatgttttaaatttgatttttttgtttattttattattaattagatttaaatttataaagtttgtagaattaataattttgagttaattatatatgaaatatgttttaatgaatttttatataattaagaatgttgttaaataattaattttaaggataatttttaatagttattttttttttttagtgagtttaaggttgttttgagttattaaagtttaagtaggtagaaggggtgtgtgtgagttaagggtgaaaagtttagaattgtgtttaattagtaaaagtaaaattttatttatataaaataaaaaaaattatttttggagatattaattttttatagtattgtttttaagtaaatttaatttttaaagaaattaaagaaagaaatttaaatatatttaaaataatttttgaaagtttttttgttttttagtataggttagttggagaggataaattaattttttttgggtttttgtatgggtgattgttttattatggagttagtgttattatttttgaatgtgtatttgtttgatattatagttaatgatttgtaatgttagtatgaagtatttttaaaatattttttttttgtttttgtttataagattgggaaatttatttgatgtggaataaagtggatgaagtagattataaatatatttgtaatttatgtgttttttttttgttttgattatttttaaattttatttgtaatttttttttattttaaatttgtagtttaaagatgtatatgagaattgttttttagtttttttttattagtattattttattttaagaataatttagttgtaagggaggaatttttttatagtaagttttaaattagtatttttgtttttaattttttattttattttattttattttatatatatagatatttgtttagagtaaaatatatttttatgtgataggtttgtattagttgaggtttatatatttagttatattaggttttgtaattttattattaaattatatatattatattagtagtttgttggtaaagaaggttaaattaatttatattttgtttattatttggtgtttaaatgatgtattttattttggagatttggtggagaattttttttttagattttatagtgttttattgaagataatgtttttatatttgtagtggtttttaatttgataagattttaatttgtttaagttttttaaataagggttttaaatgtttttagttgtttttttattgaattttttttaatttttttaagattataaagtatatgtgtaaagtaaatatttttttttattgtattgttagttgatgatttataattaagttaataagaatttagtttttttttgttgaatgtgtttattaattatattttagtttttttttttaaattttagaatagttgtggtttttataatattatgttttttaaagttttattttatgaagggattttattatattaaagaatgaaaaaaatttttattgtagttagtatatatagttttttattttttgttttttaagatttaaattttagagttgtaaatatttttggaagtttgggtgttaatgttttattttagaaagttgagaagttttatagagttatatagatttttaaatttattttttataaatttatagaattttgataaaagttttggtggttttattttattgatggaatttttattatgataaatatatatgtatgaaggattttaattagtttttaaagtggttgaaaaatttaagggtatgtgattgttttttatagtgttaatgtgtgtgagatgttggaagtattggggattagtagtagtttagatgtttaaaaagataaggtgttttaatttgtgtggatttattgaagttaagtggtgaataaagataattatttagataatttagattaaagtaaaagtaaaattatatttatttgtatatatatatttatatttattttatattatagatatatatatgtatatatatattggttttgtaaataattgatttaaagtgaggattttttttgtatttttttagtaggagttttaatatttttttaattttttaattattttatatatttatagtagtggtgattgggtgatattttttttaggttttttgtgtggtaggatattaatatgataagtttgtatggggaaaaggaggtatgtggtgggaattaagaaatattgtttagtgaaaattttgtggtatggtggtggttgattttggagatttaatgtatataagatttgtgggtgtataggtataggtagtatggatgagaaaggggttagaagaaaataaattttatgtattttgtgattttagtattattgtgatttttggttaagttttttttaattggttttagaaattattatgagtttagtttttaatatagaaatttttaatatggagaatattggtgggattttggtagggaaattagaggtgttgtatggtttatgtggggtaaagaaggaaagtttagtgttggtgtgaggttttgagtttgggagatattaggggttgttttgattggggttttttgtttatttttttaaagaaagattttagaggagggaaatgtgtgatatggggttagttgtgttttgtgttggtatttgttattgattattagttttaaagttttatttaattttatattttttagtgttagttgtgtaaagtttttttggttatggtagtgagtggttgggttgtgttgttaaattttttgtattaatttggtttgggatttaattaagtgatttttgatttttggaaagagtttgtttttagagtttatttagaagatggtttaattagatatttttttgagttgttaggttttagatgggtgggagttttgttttgtttaagttagtttaaggatgaggtttgtttggatttagtttggagttatgtgatgggtgtgagtgtgtgagtttttggtaaggtgtagaggttagatggagattttgtattttgtttgagaagtgttttattttttttaatatttggtttttttttgtatataaattaagttgaaaatagtttattatttattattttttatagttatggaattaaataatttagaaattaaaagttttattgtagttgtttttttttttattttttaaatggaatttaaaaagttttggtttgttaaaaggggaagattattttttgaattggaagtttgtagatatattgagtaatagttattttttttgggtttttgtaaatggtatttatttttttaatttatagttttagttgtttaattatttgagatttggggtaattatttgggggaatagtgtttagatggtagtgggagttattattttatagtggtttggggaagagaagagaaagagattagaggagggggtatttgttaaaattatttaatgaatatgttgttaatgtttttttatatttgtatgttattgttatagtttttttaggtgttattgagtttttagaaagtaattatttgttgaattaagtaaaataaggagaatggtatagtatatgtgtttggagaaggggaaggaagggtggaatatgaaattgagtatagatatttaggttaggaaagaaggaagtggtaaggggttaaatgaagttttatttttttgttatttttttaaataatagttggattaaatatttatttgtttttttttttttttttttttttttttttttagtttatgtttattttttttttttattttttttgtttttttttttttttgtttttttttttttagatatgttggtagttaatatttagtattagttgttatggtgattataaattatttaaatttaaaaatatttatttataatttgagatgaagtttttatttttttagtgaaataatattttaaaagttgttagttgataaaaaaaaaggaatttattttattgtagtaatttaagtaatatattatttttaaaggtttaaattaaaatgttagtttgttaaaaatatgttggtagagttttggatattttttttgttagatttttataaagaagttgattttgttatttttggttgttttttaatatatatatataattttgtatgttttttttttttttattaattttttttttttttattaattattttagtttttaaagattttatgtattgggttttaaaagaaaagaatttttttttggattagaaaatagttttattggttgttgaagtgaaagatgtggggtttagggggaaaggttattaggattataatggtggtggtggtaggaggttattttagaggagttaagaagaaaaaaaaatgtagggagaaggattggaggtggaaagatagagtaatagaaaattgagttgggtggttaggtgttgtggtgaagtttagttttgaaatgataggtatatatttttttatttttgttttttttttttttgagagaaaatttttttagttagagattttggggggtaggaggtgggtaaatgttgttgtagttgggttttttgttttttattttgggtttgttgttttttgtttattttggattatagggtatttgtttagatgaagagttattaattatttatttagttaatattaggaagatgataaagttttttatataggattaagaagatattagattgttttattagtatatttttgtttatgaataagtttttgttatatattttttttttttttgagttgttttaataattgttgtatatattagtagtgggtggtgaggaataatagttgaattagttttaagaaattttgtgtattgagttagtagtgaggaatgtgatttgtgaagattatttttgtgggtagggatttgtagggattgattatttttggataattggtataattttttttgggggtgaaaaattataatgtggtggggtattttttaagtgagttgtagatttgattggtgtggggggtgggggaggggaggggagaatgggatggtggaggttgggtggaggaaagaaaatggaaaattttttttatttttattttgttgttttttttttaagttttatgttttttttggtaagtatttgttttttttgtgttagttatagttagagttttttatttttttttatatatttttttttttttgataaggtttaggattttggttattattttatgtattattattttgtgttttgttagagatggtttgggttgattttgttggtgtttatgttaggattaaaatttttttatgatggtgaggaaaattgtattatttgtttttagggggtatattaggagtttatgtagtatatgttttgtaaatatttgttgattgaatgagaggtgtgggggtggggtggtggagaggggtttgtggttgttaaggttgttagggttaatttaggttttttgaagaaggttgggattgagttgttgttgtgtgtgaaggtgtgtgttgtggttgggggtgttataatgggttatggagtttattttttagagggaggaagtttgtgtatattagtgatttgggttgaatatttttagtttatttattgggttaaagttatttaataattaatgtgtttttgggggaggttgggggaagtatttgttttttgttgggatgtaaagttaggtgttaggtttaaaggggttgtagtgtagtttgattttagtatggaatttagagttgttgttttgaaattttttaagttagtgatagaggagggttagtttgttttttttttgagggtgaagattattttatgagttttttttaggatttttaaagtaagaaaagttaaagaaaggtttttttgttttagggggttgtttttagttggtttttatattattttttgttagttgtgtttatttttttttaaatttttggtttttaggggtttttagttgtttttgtgttatttttgttttggggttttatttaggttgggtatttgtttaatggatattaaggagaatgggatttattagggaaggaaggtagagtttggattgtttagaggtggattttgtttatatagaatgtttagtttttaatgaggatatggtatttttgggtggtgttgggggtagaggtggagagggtagtgtaatagattattatggttttttgaagaagttatatgttattgtgaattttttttttttttaaaagtaaagaaaaattttaaaaaaatataagaaataaatttttttgttttataagtaggttgtggttaggattttggatattttataagttaatttaaaattagggaaggataggtgttttattttttagtagtgttatagttttgtttatttgtgtgatttttttttgttgtttattagttttttaaaagttaattaaattaagatttttagtatttttttttattatatgtttttttttaattaatggtattaaatgtgtttaggtagtaatttttttttttggttaaaaagtagaaaaagatatattgagtgtaggggaagagttttttatgtgtattaaaataatgtgggtttgaaagtaatggttaagaaagtaattatttattatttttagttttttatgtgttagttattaaaagtgaatgattaggggtgtttgtgtggtttgtgattggtgtaagtagaatttttttgtttttagttgttttttgtttatttatgaggattttattttattgtaggtttttttatttgtttttagaatgtattttttatgtttaggaaatttggggtagggattgggggaaggagatattttgtgtttttttggtttttagtataataagaaatgttagttttggtttggtgattgtgagtttgttttgtgtgaagtgagattggggagtttttttagtttggttggagttagggttgagtttgtgtaaagtattttttttagaagttattgttgtttttgatttttaattatattttaaatatattatggtttaataatttatttttattattgattattaaatagaagaagaatttaatataatttaaatgatagaaattatgtgatgttatttttgtattgtttttatttaattttatggggttaattttggataagtgagtgtttaattggtttagtagggtgattggtggtgtagtgtagtgtttgggtgtgaagtattggattgttttagatgttttattttaataaatgattatttttttttagatttatggggaaattttaatgtaagatttttgttttttttttagtaatatggtttgtttttttgattggggttttaaattgtttttttttattttatattatatttgtatttttatattttaatttggaaagagggggttaggggttgagggttgtgggggggggggggttttatttgtttatattattaaaggttaatagttttttaaggttaggtatttatttattatggagttaggaaaatagaggaatagtaaatttgaggggtttttttttatgtatttgaaaagaaaggtatttttttttttttattttttatatttttttttttgttttatagaataagttttaatttaggaaaggtttgtggtgtaggttggagattttttaattttttatataagtttgtagattttttttggtaatgttttttgatttttttagagtgaaattagttaattaagtaatgatattgttaaaatttaaggtttggtaattagtattttaggtaggtttgtgttgataggggtaaatttttattttattttgggttgttaagtatagtggttgttttttagttttttagggatgttgttggttttttgttttttttttaattagttaaagtaaatttttgttaatttaagttttttttgtttgttttttgtgatgaattgtgtatttataagtttgggtggggtgtggttgagagtttgagtgattgagtgggttttggtggtgttgtgtgtagtgggatataatgagtgatagaggttgttgttggattatttttttatgttagtttagatgttgaggtttgttggagtgtgtgtaggggattagattatagggagtgagtgagagggagagagaggtgttgggttttaggagtgtagtataatttggggaaaggaattaatgtttttgggatggttgttttttgttttatttagaggtggagtgtttaagtttaagtagtaggtgtgttaggtttggtggttttgtttttttgtgttttgtttgaggtttagagttttggaggtgggtgtttagtgtgtggtttgtgttttttttttggttttattattggtgttaggatgttgttgtgggaagaatttgttgttggttgtttttttttggtttttaggagagtttgtgaatttgatttttttgattttggagtttttggagaagagatatttaatggttgttggttgtatgtttgggttatgtgtgtgtttgttttatgtgtggagagaggtgttttggattgtggttgaaaggagttggggatgggaggagggggaggggtgaggtaggttggaggagaaagagggataaagagtaaagatttagttagaggaaagagttgatggtatttttgttttttggattttttggtaatttggggtaggatggtgtattttttttgtgtttttttggttgttggtggttttagttgggaggagtaggttggggggttttggtatatagtgtgttgttgttttttagtttattgttttgttatagggagaggttattggtgatttggttttgattttttttttgtttaggttggttttttggggaagtgttttttgttggggttttgttgtagggttagtgtttttttgttgtttttatgtggtgtggttttttgtttgatgatttgggtaggagaagggggtttttatttaattgtatatatgttgatattagtttgtggtagttggtttttattttttgttatttgtaaaatagaagagaaggaaggttgtaagaagtggtggttgttgagtgagtagggtttagatgagattatgttatattagttgttaggtgtttattgtgtgttaggttttaggtgtgtttttttgatttgatagtttttggttgtgtagtagtatttttagtttagttttgggtttaggatatttatttattaagaggggatttttttttagagttgttgtaaaagtgtttagaggttagaggattataaagttatagtgtgttggggaggttgtggatttatttttaagaattttggtgttggggttaagaatttatttgaatgtaatggtagtgggagtgggtgggtggagaggatttttttttttgggaagttgtatgtaaagattattttttagtgtttgtttattagttggagtttggtaaatatttgtagaatattagtgttaatgtgtttttgttttagatagtagttttttttggttttttgtaattttgaaatgaatgggtttttggtttagggtgttttaggagtgagttgagtttgggttttttatttattaggagttatttttttatatttagttatatttttttttagagatattaatttggttatttatttatttattataaataattattttaaagtatgatttaagattgtagaggagagatattgggtggattgagtgagattgaggagagtagggtaaatgtttttggagggtttattgtttgttaaggatggagaaatagttttggtataattgttatttagtttttttttttttttttttgggtgagttaaattttttttatgtttttaattataatgtagtgagttaagtatttaatgtgtttttttttttttgttataggtaagttgggagaggtgggttttgaggggttttattgggtgggtagaagagttgtggttgttttaaagataagaaaagaaggtttagggttttttaggtttttttgattttagtgtttgtttttttttatgttaattagggtatgttgatgattggagggtttattttgtgtgggtgtggggattggggtgggagtaagtgttgttgggttggtggaggtatagaggtggggtagggagttgtgggtttgtttttggtttgagtattgtttttttgtgttttggttttttttgaagggagttgggttttggggagtttttggttaaggttgttgtttataggaggggttgtttggtgttgtggtgtggggatttagggtggggatggttaggtggtttttttatttgttagtgagaatgtgggtggggattttgttgatttgatttttgtgggtttgtgggtttagaagtagtagtttggtggttttagatttagtgattttgtagtaaaattataggattagtttttgattgagatgtttgtttgtgagatattataaaatttattattatagttttttattaatttgatatgaagtaatatagatgggattttattagtttagattttaaatgtttatttatgataattttggaggaaatttgtatgttattattattttgataattttttttttttatatgtttgaattggttgtattattagttggtagttggagtattgtagatggtaattgtaaatagtttttatttatttattttttttaaagaatgaaatatataaaagaaaaagattgtgttgtttggtgtaaagttagttaattattatatatttttttttttatttttttgtgttttagtgttgaagattaaataaagtaatataaaataaatttttaagaatttatagagttttattttaaggattgaaaagaaggttaaggtgtgttttttagtttatttttatatgtttttgtgatttggagatttattttgtagttaaaatgagttttgagatttgtatttttatgttttatttaatgattaggtttattagaagaattgagtttaaataattggggaagataattttttaaaaagagatttttaatttttgtttgttgatttttaaatttgttttattaagataagttttttgtgagaaatttggttgttagattttggaattggttttaatggttaattttataaattgagatgggagattttttttgatgggaggtagtttttatttttaaagtttatgttttagttggaatgtatatgttaaggatttttgttttggttaatttgggttttatattgtgagtatataaaaagtattatatggttaatggaggatgaggaattatggtaaagtaggtaggtaagttttaagaaataaaataatttgttaaaaaataatttttgatgattattgtaagattgaaagtgtaggaaaaatatagtttgaataattttagatttttttatattttttttttttttatatattttgttattttataataaaatttttaatggaaagtttaaaaataaatagtataggaatatgtgttttaaatgaattaaattgtgaaattagttagtaaattaatttgtagtaagtaattatttaaggaaattaaaatattgtttagtttagttttgtattttattatgtgtatgtgttttttataattaattaatataagtgttttaggaatatttgaagataaatatgtttaatttaaggaataaagtatttaaataatttaagtgtaattttgttgagttaaagtaaaatattttataaatgaagtggttatttaattttttagggaaagtttggttattgaaatgttgtatgtttatgttatattaataaaaatttttaatttattttgtttatgtgttttgtttttttgatattattggtatttgaattttagatggatttttgttaaaatgatattttgtgtgataaaagtatttttagttttgattgatagattaaaataaatgtaaggaaatttttttaaattagattaattttttataaaaatattttagaatgtatgaattttgatatttatatttataatggtaaaagttttttttgtttagtttagtaagataatatttatataaaagagtaaaaaaaaattatattattttatgatagtttgatttttaaattgtttaagaaagtaaagtggttaaattggaaaagaggaatatattttggaggtttagaattgaaaattttttttttaatttttagttggaaaataattttttgtatttatttaaagtgtattttttgaagtgttagattggagttgattggtgattaatttaaaggagttataatttaaagaaatggtgagagtttggtatttaggtttggtttttaggtaatttgtttgggtttgagaggttattaattgttagttaagatggaattttttttttttttttttttttttaatggataataatgggaagggggttaattttttagtagttgaaattttgtatttagttttttattttgagaatgttaatttttggtttgaggatttgtttttgtagtgttggtattgagatttaagggaagatattttgttttaaatgttagttatggtttggttttttttttgattttagtattttgtagattgttagtgtttgtggtgggggatgaaaggaatagggttttgtaaggtttgtttgttgattgtgttattttgggtgaaatttagttttaaaagttataaattatttatggtgaagattttttgaagtggaataaatttttagatttgtattattttatatttttgtgggatagatggtttttatttattggttattgggagagagttgttgtttttgtgttttattgttttttggggtgatttttagtgagttgagtttttggttgtatggtaagtgtttgaaagttgggtttgagaggattgtagggtttttgagggtgttaagttttgaaggagtttatgggtgtattggggtttttgaaatttagttgttattggtagtttttttttgtttttttttagtttttttgtttggttttgtattttttttttttttttttttttttattttttttttttttttttgtttttattttgtgtggggagtgatgtgatgttagtagagattttattaaattttattgtatagtggtgtgtgggtggttggttgagtttggttgtgtggttggtgatttaggagtgagtatagtgtttgggtgagtgttggggggagtgagtaggggtgatgagaaatgaggtaggggagggaagtagatgttagtgggttgaagagttgggagttggagttgggagagtgaaaggagaggggatttggtggggtatttaggagttaattgaggagtaggagtatggatttttattgtggaaaggaggattagaagggaggatgggatggaagagaagaaaaagtaatttgtgttaatttggtagttttaataaattaaagggggagtgttagggtagtggggagatagaaatgtatttttggggagtaaattaggatgggttgggaggaagtgatagggaaagtggtttaagagatggaataaaggataatgtttatggggttgtttgggatgaggtgtgtggagtgtgggtgtgagtgtgtgtgtgtgatttttttttaggtttgtagagttgaggaaagaggttatagtaaagagggattgtggagggaggaaagtgagagattggtagagggtgggagtggaggtgggtgtggtggggatgggagaggatgagtgaagagaaatttagaagaatggagtgagttagtgggagagggtgggagggttatagttgggagtgaatgagttaggtttgttagttggggaaggttgggatgttgggtttagtttagttgggatattgtgtttgaggttaaggtgggtggattaggtatgttgagagtgttggtgtataggtgggtatggttatgtattgatttagtgtttatgaagggtttgtattggataaggtttagatgtttatagagtttagaattttttttgttgtatttatatttaataagtttattttgggttatggatattttattttttaaaatgatgaggttaaggtttttggtgaggatggtattaaattgtatgggatagaagtgggggtgggggagagagttttttttaagtttatatttgtttttgtaaagtaaagagtatgtgaaattatagggtatatttttatttgaaaagtgtgttttatttttgaattttgattttttgattttttgatttgagtaaagatgtgtattttggtagtgagtagaatattttggttttgttttgtttttgagtggaaggattataaatataatttgtttggaggattaggtgtgaaggtttttgttaggtatatgggataatgtttttttaattttaagggtattttgttaatgtatgtttttggaaagtgttggaatatagttattgtttttggatttggatttttttattaatattaatttttgtttgagagtaaaatttaggtttgttattaaaaagatatttttttggtttttaattgagaataaagttttttttaaaagttgtattgttttttttaaattaatatattaatatttgtaattttagaaatatatagtgatttgggagaatgtgtataaaatagatatgtttaaaaaagtttggtgtttaaaattaattttagttattatataggtgttgggttttttttatttttgggggttgtttggaatatgttatgtgtttttttgaattattttgtgttttgaatttatttgagttagtagtaaaaataggtaaataaatttgtttaatttgttttgagtgttaaatttttttattttgaaatagttaatagttgatagatggatttattttatggaaagggttagtttttttagttatgaagaaaattgattagagatttatattttaagttatttttaatttttatgtaatatttgtgaaaatttaaattttttttttttatttagtggaaatttaaagtagtgttatttaaggggagagaaatgagggggaaaatgtttatgtgttgtttaattgtattttttttttgattttgagaatttttatttttggtttttgaaattttgttgaggtaagaaaattaaatttttttaataagttttataattgaattttagttataggatattggaaagtgtagtttgagaaagatatttttatttttgtttattgatgatttttgtagtttttttatttttttgagtaatgggttaataattttttttttttttttttttattttgtagagattaagaggtgtttgtagtagaatggttttgtttttagttggtggtgaggataggtaattttatggaaaagttggaagagaatgagaaaattaaagatagaaagatttagagatttgtggagagatatagggagagggaagggagttgtgttgaaaagatgtaaagatatgtgtgtgtaattttttttttttttaggttttagaggtttgtaaattagggttgagaggaaggggtttgggaagtttatgttttttttgttttttttttgtttggagttttgtttgttagaggttggttaattttagttttggttgttgtagatattgtgttgagtttttgggtttttgttttgtttagtgttagtgtagttgaagtgagtagttggtgggaaatgtaaatggtttttggagaaatagaagatatagaatgatttttattttttttttgagtgtgtggaaggagttggatatatgttttatgtttttaattttttttttatatttttagttatatttttattaaataattaattaatgtttagaattattagggaatatattaggtatgtaattgtagaagtagggtgttggggggttataaattattgagttgatttaagatgtggattttaggttttttttttgttaaagtagtaaaggaagagtgggttttggtgattgtatttagattttgattattttaaattagaagggggtggagggagtgtttaagtaaagtaagtaattttttgttttgtagatgtaaataagattgtagtattaaaggtattagtttttttagggttagattgtttggattgggagtttggggaaggggagatattaattttatgtatttgtgaattttaaggatgttatatttttatataaataattttagtgtggattttttggaatggggggagtaatatttttattttagaatattaaaatattttttttttaaagtgtatattttttttatttttttaaaattttgaattatgtttaaagataatagttttttagtaaattggagtattggattattttttttatttttttttattgatattttgatgatttgattttaatgtgtggggggtatagggaattaaatatagtttataaaattaagtttagatgaaatagtgttggttaagtgggtttagataatttttaatgagaattttaattatatttttttttttaatatgttgagataagtgatagaattgttagaatggtaattaaattggaaagtttagggagaataataattttgtgattaaattggggtaaaattgtggataaatgtggggtgatttttgttaattttttgttatttaagagttaggatttgggaaaggtatagtattattttagagtttgttgtgatgggttgtgtgttattatttattttttttattttggattatgattttaattttggtaagtaatttttttagttttttatttgataataagtgagtatgtaaatattaatggttagtgatgtttaattgttttaaatattattgatttgttggttgttttaaattgtttttttagtttaggttttgtttttgaattgtttattttagaggtttgatttatgtttttgatgttataatataataattgtttttttaaaaaaggtatttaagatgaattaattgatttgtatataaattaaaattattatgtgttgttgattttggtgttttataattattttgaaattagtatttaattatttgagttaaaagaatatataaatgtttgtattgatttattaatgaattatttaattaaaatgtttgggtaatgttgggtgttggaaagattgttaaattaagatatattataggagggatatgaagattagaaaggtaatagattaatattttgtatttaaaatggagtttttggtgatttttagttttaattttggagtaggggttttttttttttgttgttaaaaagattttgtgtttgtttgtgagtgagtgtatttaagtggaaggaatgtttttatggttatggtggtttaggttttttgtttggattgggattttatagttttaatttaggagtgttaaattttggaagattttgggttagttttggaggtgtgtggttttgtaagttgttaggttaagtttgttttttttgtttgtttttttggtaggttgggtgtgttatggtagtgagttttttgtgtaaatggagagttggaattaaagttgatatttaatagatatgttaattgagtatttatttttgttttgagaataggaataaaaggtagttttttttaagagaggtggtgtaaaggtatgttataggagtttagaaaaggttggtggtgggaaatttgtagtttgggggttagttaatatttttttttattttaagtatttattgatttgttgttgttatttttggtgatgtagaaggatatttgaaagaatttttgatggggttttgatttgagaaaggaggtgatttgtttaggtttttattaaatttttaattattatattaattgtttttttttattttttatttgatttttttttttttgtttatttttaattttttaattatttagaaatttttttattttttagtggttttttttttgtagtagttttttatttgaattttttttttgtttttttgtggtagggtttgtatattgatttttttgatttttggtatatttgggttttttgaaattttttaatttttttagatttgaggatggtaggttttattttttttattgtgtgtatatatttagagatatgaaaatttatatagattgtttttaaatttagggtatttaatagatgttttttttttagtttgttttttgatttgaaatgtttgtttgattttaatttggatattatttttttttgtttttttttttttaaagtagtttggatatgtgtgtaagtgagtttagaatagttttatttatattttttattaaattgtaaataaaagaagaattaatgaagtagattggtatatagattgtattaagagtttgaatttttagtttttggattttttatttaattttggttgttatttatattgatagagttattttaagtagaggtttagagaaatttgtattgtgggataataggtaaagttatagtaaaaagtggaataattttaaagttattttattagaatgtaaattgtatttttgggttttgtttgtaattatttagttttaatatatatagagttagataggaaaaaataggttaatatagttattggtattagagaagataaattttatgggttttttagtgaaaagaagatttttaaagtttataatttttgattatttaattttatttataattgtgggaatgaataagatattaattgttttatgtattttatttatattaattaatttgtgtttttattaaaagtagttatatagaattttttttaatttttggtagtaagtttagaaaatgaagtttatagttattttgaattggatatattttttgagttgattatttttgtaagtgtaggaatataatattgtttttttatggtttttttgtatttttttagggtttgtaagtttttattaggtttgatattattgtttgggtttatatttattataagtaaatttgattattatgttgattttaaaatagtttatttggttagtataattttagtttttaaattataaaaattttttaatatatgaagtttttagtttttattttttttagttttttgtttatttaaaatttttattttaattggtgtaagtaataataatttgtattattatttgtattttttttatttttttggagattgggttggattttagagagaatattagtattattattattataaataataaaatttaaaagtaaagtttttatttgtatgataattggtatttggaatgtttttgatttatttaatgttattttataaaggtattttgtaaatttttttggaatttttagtaagagtttgtagtaattggaataattttttgggaagatattttttttgatgggtttttagtttttggaggaatagattgagagtaattagggagggaggggatattggaaattggtagttatgttagttgaaataagtttgggtttagtaaggtgattgatgttgtggttgattttttattttgagtttttttttaattggggtattgattttttttattttgggattttaaggtatttggtgtgtatgtagattttttttttgtggtttttattatgtggttttgtagtaggtttttggtttaatgatattttatagttatagtttttatatttattattatgattttaatgtttaggtttttagtgtatttatattaaatttgttttattagtaagttggagtatataggagagatgggggtaagtaaggatttagtagagtttaaatttagatatgtttaaatggttttgattgtgtaaagtgtggtaatgttttttgttgttttagttttttattttaagttttatatgttttttggttaatgaagtgtgatataggttatatgttaggaataatagtatttgttgagaataaagtgaatttaggaaatttggtatatataaaatgtatt

SEQ ID NO: 5 - DNA, 28536 bp, Artificial Sequence
  chemically treated genomic DNA (Homo sapiens)
gatgtattttgtgtatattaggttttttgagtttattttgtttttaataaatattattatttttagtatgtggtttatattatattttattagttagaggatatgtgaagtttagagtggaaagttagggtagtaggaagtattgttatattttatatagttaaagttatttaaatatgtttgaatttgagttttgttgagtttttatttgtttttattttttttgtgtgttttagtttattaataaagtaggtttaatataaatatattaaaagtttaaatattgagattatgataatgaatatgagggttatgattataaaatattattgaattagagatttgttatgaaattatatggtgaagattataagggaggaatttgtatatatattgagtgttttgggattttaaagtgggaagagttagtgttttaattaaagagaaatttggggtagggaattaattataatattagttattttattgaatttaggtttgttttagttgatgtagttgttaatttttaatgttttttttttttttaattgtttttaatttatttttttagaaattgaaagtttattaaagaggatatttttttagagagttgttttagttattataagtttttgttagaaattttaagaaagtttataaggtatttttataaggtggtattgagtaagttagaagtattttaagtattaattattatgtaaataagggttttatttttagattttgttgtttgtggtggtggtgatgttggtgttttttttggaatttagtttaatttttaaaaaagtaaaaggagtataaatagtaatataaattattattatttatattaattaaaatagaagttttgaatgagtaaggagttagaggaggtaaaaattgggaattttgtatattaaaaggtttttataatttgaaaattaagattatgttggttaaataggttgttttaaaattagtatagtaattaaatttgtttgtaatgaatgtagatttaaatagtgatgttagatttgataaggatttgtaaattttaaaagagtgtaaaaagattatagaagaataatattatatttttgtatttatagaaatggttagtttaagagatgtatttagtttagggtggttgtaagttttattttttgaatttgttattagaagttaaaagaaattttgtataattgtttttgatggaaatataaattggttgatgtaaatgaaatatataaagtagttggtgttttatttatttttataattataaatgaaattaaatgattaaaaattatagattttggggattttttttttattgaggagtttatggaatttgttttttttagtattaataattgtgttgatttatttttttttgtttaattttgtatatattaaaattaggtggttatgaataaaatttagaaatataatttatattttaataaaatgattttaaaattattttattttttattgtggttttatttgttgttttataatgtaggtttttttgggtttttgtttagaatgattttgttaatgtagatgatagttagagttgaatggggaatttagaaattggggatttgggtttttgatgtaatttatatgttaatttattttattagttttttttttatttatagtttggtaaagaatatgggtggagttgttttgggtttatttgtatatatgtttaaattgttttgaaaaaggaagggtaagaaagagtggtatttaagttggaattaggtaggtattttagattaagagatgaattggaaagggaatatttgttagatattttgggtttgaaggtagtttgtgtaagtttttatatttttgagtgtgtgtatatagtggagagggtggagtttgttatttttaaatttgaaaagattgagagattttagagggtttagatgtgttaaaggttagagggattaatatataggttttattatggaaaggtggggaaaaggtttgaatagaaaattgttgtagaagggaagttattgagaggtaagggagtttttgaataattaaaaagttaagaataagtaaaaggaaggaggttgggtgggggataaaaaaaagtagttgatgtggtaattaagaatttggtgggagtttgggtaggttatttttttttttagattagagttttattagaaatttttttaagtgtttttttgtgttgttaaagatgataatagtaaattaataagtgtttgaaatgaaaggggatgttgattagtttttaggttatagattttttgttgttagttttttttgaatttttatagtgtgtttttgtattgttttttttaagaagagttattttttatttttatttttaggatgaaggtaagtgtttagttagtatatttattaaatgttagttttggttttagttttttgtttgtgtggaaagtttattgttatagtgtgtttagtttgttggaggggtagatagaaaaagtaagtttggtttggtgatttgtggggttatgtatttttagggttggtttggagttttttagagtttaatgtttttgggttagaattgtaaggttttggtttgagtaaagggtttgagttattgtagttgtgggagtgttttttttatttgaatgtatttatttataaataagtataaaatttttttaatagtagaggagaaagatttttgttttaaaattaaagttgggaattattggaaattttgttttgagtgtgagatattggtttgttatttttttaatttttatattttttttgtaatatgttttgatttaataatttttttagtgtttagtattgtttggatgttttaattgggtaatttattagtgagttaatataggtatttatatatttttttagtttaagtggttaagtattaattttgaaatgattataaaatattggaattggtaatgtatagtaattttaatttatatgtaaattaattggtttattttaaatgttttttttaaaaaaataattattgtattgtagtattggaggtatggattaaatttttagaatagataatttggaaataagatttggattaggaagataatttagaatagttaataaattaataatgtttgaggtagttaaatattgttagttattggtatttatatatttgtttgttgttagataaggagttggggaaattgtttgttagggttgagattataatttagagtgaagaaagtaaatggtagtatatagtttgttatagtgggttttgaaataatattgtattttttttaaattttgatttttgggtgatagggagttggtggaggttattttatatttgtttatggttttgttttaatttgattatgaaattgttgttttttttgagttttttaatttgattattattttaatggttttgttatttgttttaatatattggggggaggagtgtaattgagatttttattaaaaattatttgaatttatttagttagtattgttttatttaagtttagttttatgggttgtatttaattttttgtgttttttatatattaaaattagattattaaaatgttggtaggaaagggtgaaggaaatggtttaatgttttagtttattggaagattattatttttagatatagtttaaaattttgaggaaataaaaaggatatatgttttggggggaaaatgttttaatattttagaatgggggtattattttttttattttagagaatttgtattggagttgtttatgtaaaaatgtaatatttttgaaatttatagatatgtaaggttagtgtttttttttttttaggtttttagtttaggtgatttagttttaaaggagttagtatttttgatgttataattttgtttatatttgtagggtagagaattgtttgttttgtttggatgttttttttatttttttttaatttgaagtaattggaatttaaatatagttgttaaggtttgttttttttttattgttttgataagggaaaaatttgaaatttatgttttaaattagtttggtggtttgtagttttttagtattttgtttttatgattgtatgtttaatgtattttttggtgattttgggtattaattagttgtttaataggagtatgattaaaaatgtaaaagaaggattaggagtgtgaaatgtatgtttagttttttttatatatttgaggagggaatgagaattattttgtattttttatttttttaggagttatttgtattttttattagttgtttattttagttgtattggtgttgggtaaggtgaggatttaaaagtttagtgtagtgtttgtggtggttgggattggggttaattagtttttggtgggtgagattttagatagaaggggggtgagaggaatgtgagttttttgagttttttttttttagttttggtttgtaaatttttgaaatttgaaaggggagggagttgtatgtgtgtatttttgtgtttttttagtgtaatttttttttttttttttgtgtttttttgtggatttttgaatttttttgtttttggtttttttatttttttttaatttttttatgagattgtttatttttgttattagttgaaggtaaggttgttttgttatgagtgttttttaatttttataaaatgaaaagaaaaaaagggaggattattagtttattatttagaggaatggggaggttgtaaaaattgttgatgggtagaggtgaagatgttttttttggattgtattttttggtgttttgtaattagagtttagttgtgggatttgttgaagaaatttgatttttttgttttggtgagattttaaaaattagaaatagaaatttttagagttagagaggaaatataattaaatagtatgtgggtatttttttttttatttttttttttttaaataatattgttttgagtttttattgggtaaagagagaaagtttgagtttttatggatgttatgtggaggttagaaatggtttaaaatgtagatttttaattagttttttttgtggttgaagaggttaattttttttataaaatgagtttatttgttgattgttagttattttaaagtgaagggatttagtatttaaaataaattgagtaagtttgtttgtttgtttttattgttaatttaaatgaatttaaaatatggagtaatttaagaaaatatataatatgttttagatagtttttaaaagtagggaaagtttagtatttatatagtgattagggttagttttaagtgttaagtttttttaaatgtatttattttatgtatatttttttgagttattatatatttttaaaattgtgagtattggtatattgatttaggaagagtaatataatttttagagggaattttatttttaattagggattaaagagatgtttttttaatagtgggtttgagttttgtttttaagtaggaattaatattggtgggaaaatttgaatttaggagtaatggttgtgttttggtattttttaaaaatatatattaataggatgtttttgagattgaaaaaatattgttttatatgtttggtagaagtttttatatttggttttttaggtgaattatatttatagtttttttatttagaggtaggatagagttaaaatattttgtttattattaaaatatatatttttgtttaagttaagaaattagaaaattagggtttagaagtaaggtatattttttgagtgagaatatgttttgtaattttatatattttttgttttgtaggagtaaatgtggatttgagggaaatttttttttttatttttatttttattttgtgtaatttaatattatttttgttaggaattttaattttgttattttaaaaaatgagatatttgtgatttagggtgaatttgttgaatgtaggtatagtagaggaaattttagattttatgagtgtttgagttttgtttagtgtaaattttttgtgaatattgggttagtgtgtggttgtgtttatttgtgtgttgatatttttagtatgtttggtttatttgttttgattttgggtgtggtgttttagttaagttgggtttagtgttttggttttttttagttgataagtttagtttgtttgtttttggttgtggtttttttatttttttttattagtttattttatttttttagatttttttttatttatttttttttatttttattgtgtttatttttatttttgttttttattggttttttatttttttttttttgtagtttttttttgttgtgatttttttttttaattttgtaggtttgaaagaaggttatatatgtatgtttatatttatattttatatgttttgttttaaataattttatgaatattgttttttgttttgttttttgggttattttttttgttgtttttttttagtttgttttgatttgttttttaaaagtatgtttttgtttttttgttgttttggtgttttttttttgatttattagggttgttgggttggtgtagattgttttttttttttttttattttatttttttttttggtttttttttttatagtgggagtttgtgtttttgttttttggttggtttttaagtgttttgttaggttttttttttttttgtttttttggttttggtttttgattttttggtttgttggtatttgttttttttttttgttttgttttttgttgtttttgtttgttttttttggtgtttgtttgggtgttgtgtttgtttttggattgttagttgtgtagttgggtttggttggttgtttgtgtgttattgtgtagtggagtttggtggaatttttgttgatgttatgttattttttatatggagtaggagtagagggaagagagagggatgagagggagggagaggagagagagtgtgagattgagtgagaaagttggagaggagtagaaagaaattgttagtggtggttagattttggaggttttagtgtatttgtggatttttttggaatttggtatttttaggagttttgtagtttttttaggtttggtttttgggtgtttgttgtgtagttggaggtttggtttgttggaaattgttttgggaagtagtgggatgtggagatagtagtttttttttggtagttggtaagtggaggttatttattttgtagggatgtgagataatgtgagtttggaaatttgttttattttggagaatttttattgtaggtgatttgtggtttttggggttaagttttgtttaaggtaatgtagttggtaaatagattttgtaaagttttgttttttttgttttttgttatagatattaataatttatagggtgttgaagttgagagggaagttagattgtggttggtatttaaaatgaggtatttttttttaaattttggtgttaatattgtaggaataaatttttgggttaaggattagtatttttaagataaagggttgggtataaagttttagttattggaagattagttttttttttattgttatttattgggaaaaaaaagaaaagaaaaagattttattttaattggtagttagtgattttttaggtttaagtgaattatttgggagttaggtttggatgttaagtttttattatttttttggattgtaatttttttaaattgattattagttaattttaatttggtattttaggagatatattttaaatggatgtagagaattattttttagttggagattaagaaaaaaatttttgattttaaatttttgaaatatgttttttttttttagtttaattattttatttttttaagtaatttagaaattaaattattataaggtggtgtgatttttttttatttttttgtgtgagtattgttttattaaattaaatggaaaaaatttttattattataaatgtaaatattagaatttatatattttaaaatatttttatgaaaaattaatttgatttaaagaaatttttttgtatttgttttagtttattaattaaaattaaagatgtttttattatataaaatattattttggtagaaatttatttaaaatttaaatattaataatattaagaaaataaagtatataagtaaaataaattgaagatttttgttgatgtaatatgagtatataatattttaataattaaattttttttaaaaaattaaatagttattttatttgtggaatgttttattttaatttagtaaaattatatttaaattatttaggtgttttgttttttaagttaagtgtgtttgtttttaaatgtttttaaagtatttatattaattggttgtaaagaatgtatatatatggtaaaatatagaattgaattgagtagtattttaatttttttaaataattatttattataaattaatttattggttaattttataatttagtttatttaaaatatatgtttttgtgttgtttatttttaaattttttattaaagattttgttatggggtaataaagtgtatgaaaaggggggaaatgtgaaaggatttgggattatttgaattgtattttttttgtatttttagttttgtggtagttattagaaattattttttagtaaattgttttattttttagggtttgtttgtttgttttgttatggttttttgttttttgttagttgtgtagtgttttttgtgtgtttataatataaaatttaagttggttaaaataagagtttttggtatatatattttaattagaatatgaattttgggggtgagaattattttttattaggaaaagttttttattttaatttgtgagattagttattgaagttagttttgaagtttggtagttaaattttttatagaagatttgttttgatagggtaagtttaaggattagtaggtgggaattggaggtttttttttaaaaaattattttttttagttatttagatttagtttttttagtaggtttggttattaaatgaagtataaaaatgtaagttttaaggtttattttgattgtaaaataaatttttaagttataaggatatgtaggagtgagttaaggaatatgttttgattttttttttagtttttagagtggagttttatgagtttttgaagatttgttttgtattgttttgtttggtttttagtattgaagtatggggaagtggggggaagaatgtgtaataattgattgattttatattaagtaatgtaattttttttttttgtatattttattttttaaaaaaaataaataaataaaaattatttgtagttattatttgtagtgttttggttattagttaataatgtagttagtttagatatataaaaaaaaaagattattgaaatgatgatgatatgtaaattttttttgaaattattataagtaaatatttgaagtttggattaataaaattttatttgtgttattttatattgagttagtagaaagttgtgataatgaattttgtaatattttatgaatagatattttaattagggattaattttgtgattttattgtagaattattaaatttggagttgttaaattgttatttttgggtttatgggtttataaggattgaattggtagagtttttgtttgtgtttttgttagtgggtgggggaattgtttggttgtttttattttggatttttatgttatagtgttgggtagttttttttgtaggtagtgattttggttagaggttttttagggtttagttttttttaggagaggttgagatgtagggaaatggtatttaggttagaggtaggtttgtagttttttgttttgtttttgtgtttttgttaatttgataatgtttgtttttattttgatttttgtatttgtgtgaagtgggttttttggttgttggtgtattttggttagtgtggagagaggtaggtgttgagattgaaggggtttagggagttttggattttttttttttgtttttaaagtaattgtggtttttttatttatttggtggagttttttgagatttattttttttggtttgtttgtggtagagaagggggagtgtgttaaatgtttggtttgttgtgttgtggttgaaaatgtgaaaaagatttggtttgtttgggagagaaagggggagaattgggtagtagttatattagagttatttttttgtttttggtgggtagtaaattttttaagaatgtttgttttgttttttttagttttgtttagtttatttagtgttttttttttgtgattttaaattatattttagggtaattatttgtagtaagtaaataaatggttgggttagtatttttaggagaaagtgtggttaaatatggaaaagtggtttttgatggatgagaggtttgaatttagtttgtttttgaaatattttaggttaagagtttgtttgttttagaattatagaaaattgagggaaattgttgtttaggataggggtatgttggtgttgatgttttataaatgtttattgagttttaattaatggataagtattgaagggtggtttttgtatatagttttttaaagagaaaagttttttttatttatttatttttgttgttattgtgtttagatgagtttttaattttggtattgagatttttgaaagtaggtttatagtttttttagtatattgtggttttatagttttttaatttttgggtatttttgtggtaattttggagggagatttttttttgataaataaatgttttgggtttgaggttaggttggagatgttgttgtatagttagaggttgttaggttggaaaaatatgtttgaagtttagtatatagtaggtgtttaatagttagtgtaatgtagttttatttgagttttgtttatttgatggttgttgttttttatagtttttttttttttttgttttgtagataatggggaatggagattaattgttgtaaattggtgttggtgtgtgtgtaattaggtaagaattttttttttttgtttgggttattggatgggaggttgtgttatgtgagggtggtaagagggtattggttttgtggtgaggttttagtgaggggtgtttttttgaggggttagtttgggtaggaaggaaattagaattaaattgttagtggtttttttttgtggtggggtggtggattaggaagtagtggtgtgttgtgtattgaagttttttagtttattttttttggttggaattgttggtaattggggaggtgtagaaagagtatgttattttgttttgggttgttagagggtttgggggatggggatgttgttagtttttttttttaattgggtttttgttttttgttttttttttttttttggtttgttttgtttttttttttttttttgtttttggttttttttggttgtggtttgggatgtttttttttgtatgtggggtgggtgtgtgtgtggtttaggtgtgtagttggtggttgttgaatgttttttttttaaagattttgaaattaaaaaggttgagtttatggatttttttgagagttgaaaagaggtagttagtagtaagttttttttgtggtagtattttggtgttaatggtaaggttgggagggaagtgtaggttgtgtgttgggtatttgtttttgggattttgggttttgggtgaagtgtaagaaggtgaggttgttagatttgatgtgtttgttgtttgaatttagatattttgtttttgggtgggatgggaagtagttgttttagggatgttaatttttttttttaaattatattgtatttttgagatttaatattttttttttttttttgtttgttttttgtggtttgattttttgtgtatgttttagtaaattttggtgtttaggttggtgtggaaaagtggtttaatagtgatttttgttgtttgttatattttgttgtgtgtagtattattagggtttatttagttatttaggtttttagttatgttttatttagatttgtgggtgtgtggtttattgtgggaggtaagtaagggaaatttgagttggtgaaggtttgttttggttggttgggggaggggtggggggttgataatatttttgaagagttggagggtagttattgtgtttagtagtttagggtagaatggaggtttgtttttgttgatgtgaatttgtttgaagtattggttgttaggttttgggttttggtgatgttgttgtttgattggttggttttattttggaggaattgagggatattgttagaggaggtttataggtttatgtaaaaagttaaaaagtttttaatttatgttataggttttttttgaattgaaatttgttttatggggtggaggggggggtgtaagggatggaggagggaagatgtttttttttttaaatatatggaaaaaaattttttaaatttattgtttttttatttttttggttttgtagtaaataagtgtttagttttaggaggttattgatttttgataatgtgagtagataaagtttttttttttttatagtttttggtttttaattttttttttttggattaaagtgtaagaatataaatgtaatatgggatggaggggggtgatttgggattttggttaaaaaaataaattgtattattaagaagaaaataaaggttttgtattggagtttttttgtgaatttgagagaaaatgattatttgttgaaatgaagtgtttaaagtgatttagtgttttatgtttggatattgtattatattgttagttgttttgttgggttagttaaatgtttatttgtttgggattaattttatggggttaaatgggggtaatgtagagataatgttgtgtgatttttgttatttagattgtgttaaattttttttttgtttgataattggtagtaaaaataaattattagattgtagtatgtttgggatatggttaaaaattaagagtagtgatgatttttggggagaatgttttgtgtgggtttagttttggttttggttagattagaggagttttttaattttgttttgtgtggggtgggtttgtagttgttaagttgaggttgatattttttattgtgttgggagttagagagatgtaaaatgttttttttttttagtttttattttaggttttttagatatggggaatgtattttgaggataggtggagaagtttatggtaggatggggtttttgtaggtgagtaggaaatggttaagagtagaggagttttgtttgtgttagttataagttgtgtaggtgtttttggttgtttgtttttgataattagtatataaagaattagaaataatgaatgattgtttttttaattattatttttaggtttgtattgttttagtgtatgtgaaaggttttttttttatatttaatatgtttttttttattttttgattgaaaagaaaaattgttgtttaaatatgtttaatgttattaattaagaaaaggtatgtaatgggaagaaatgttgaaaattttgatttaattggtttttaaggaattagtagatgataaaaaaaaattatatgagtgggtaaagttatagtattgttgaaggatagagtatttatttttttttgattttaagttaatttatggaatatttaaagttttggttatagtttgtttgtaaaataaaaggatttattttttgtgtttttttaaagtttttttttgtttttaaagagaaaaaaagtttataatgatatatgatttttttaaaaggttgtgatagtttattatgttattttttttgtttttgtttttaatgttgtttaaaaatattatgtttttgttaaagattaaatgttttgtataggtagagtttatttttaagtagtttaggttttgttttttttttttagtgagttttattttttttggtatttattgggtggatgtttagtttggatagaattttgaaatgggggtagtatgagagtgattggagatttttaaaagttagaggtttgagagagggtggatgtagttagtagaagatggtgtagaagttagttgagaatgattttttagagtaaagagatttttttttggttttttttgttttgggggttttgaaaggaatttataaaatggtttttatttttaggaggaggatggattgatttttttttgttattggtttaaaaagttttagggtggtggttttgggttttgtgttgaaattggattgtattgtagtttttttggatttgatgtttggttttgtgttttgataaggggtgggtattttttttggttttttttaggaatgtattaattgttaaatagttttggtttagtggatgggttgaaagtgtttgatttaagttgttggtgtgtatagattttttttttttgggaggtgggttttatggtttgttgtggtatttttagttgtgatatatatttttatatgtggtagtagtttggttttaattttttttgaaggatttgggttaattttggtggttttggtggttgtagatttttttttgttgttttgtttttgtgttttttatttaattagtgaatgtttgtggagtatatattatgtggatttttaatgtattttttgaaagtaaataatatagttttttttgttgttatgaagggattttaattttaatatggatattagtgagattagtttagattgtttttagtaaaatgtaaaatggtggtgtgtggggtggtgattaaggttttgagttttgttagaaagaaggggatgtgtagagaaaggtggagaattttagttgtggttagtgtggaagggataggtgtttgttgaagggggtatgaggtttgaggaaaaagtaatgaaataggggtaaggagagttttttatttttttttttttgtttgatttttgttattttatttttttttttttttttttattttttgtgttaattaaatttgtagtttatttgaaaggtgttttgttgtgttgtggttttttatttttaggggaaattgtattagttgtttgaaagtagttagtttttgtggatttttgtttgtaaaagtggtttttataggttgtgttttttgttgttgatttggtatataaagttttttaaggttggtttggttgttattttttattgtttgttgttaatatatgtagtagttgttagagtggtttgggggaaaaggaaatgtataatgaaagtttatttgtgagtaggaatatattaatggaataatttgatgtttttttaattttatgtaaaaagttttgttgtttttttaatattgattgaatgggtaattaatggttttttatttaggtgaatattttgtaatttaagataggtaaaagataataagtttaaggtagaagataaaaggtttaattgtagtggtgtttgtttgttttttattttttagggtttttgattaggaaagtttttttttagaggagaaaaaggtaggagtgggagaatatatatttattattttggggttagattttattgtagtatttgattatttagtttagttttttgttattttgtttttttatttttagtttttttttttgtatttttttttttttttaatttttttaggatgattttttattattattgttattatggttttaataatttttttttttaaattttatattttttattttagtaattaatgaggttgttttttgatttaggaggagattttttttttttagaatttaatgtgtagagtttttgagaattaaagtagttggtaggggaggaagaaattaatagaaagggagagagtatatagaattgtgtgtgtatgttaaagagtgattaggaatgatagagttaatttttttgtgaggatttgatgggaagagtgtttaagattttattagtatgtttttaataggttgatattttaatttaaatttttagaagtaatatattatttgggttattataatgaggtgggtttttttttttttgttagttgatagtttttaaaatattattttgttagggaaataaaagttttattttagattataggtgggtatttttggatttaggtgatttatggttattatgataattaatgttgaatgttagttattagtatgtttgggagagagaaaatagaaagaagggagagtaaaagaaatagaaaagggagatggatataagttggagagggaagaaaagagaaaaagaggaagatagatgagtgtttaatttaattgttgtttaaaaaagtggtgggggggtgggattttatttagttttttgttattttttttttttttgatttggatatttatgtttaattttatattttatttttttttttttttttttaaatatatgtgttatattattttttttattttatttagtttggtaagtagttgttttttggagatttagtgatatttaggaaaattgtggtagtaatatgtaaatgtgaggaagtattaatagtatgtttgttgagtgattttagtaaatgtttttttttttaatttttttttttttttttttttaggttattgtgaggtggtaatttttattgttatttgaatattgtttttttaggtagttattttaaattttaaatggttgagtagttagagttgtgggttggaaaaatgggtattatttgtagggatttagagagggtggttgttgtttaatatatttatagatttttaatttagaaaataattttttttttttgataagttagagttttttaaattttatttaggaaatggggaaaaggatagttatagtgaagtttttaatttttgggttatttggttttatagttatgaggggtggtgggtagtggattgtttttagtttggtttgtatgtagagaaaagttagatattggagggggtggggtattttttgggtaggatgtaaggtttttatttgatttttgtgttttattaggagtttatatatttatgtttattatgtggttttaagttgagtttaggtgggttttgtttttgagttagtttgggtagggtaggatttttatttgtttaaggtttaatagtttagggagatgtttaattaagttattttttgggtgaattttgaagatagattttttttaaaagttagagattatttggttgagttttaggttagattgatatggagagtttggtggtatagtttaattgtttattgttatggttagagggattttgtataattaatattgaagagtgtgaaattaaataagattttaagattggtaattggtggtaaatattagtataaaatatggttgattttatgttatatattttttttttttagggtttttttttgaaagaataagtaagaaattttaattgagataatttttgatgttttttagatttaaaattttatgttggtattgggttttttttttttgttttatgtgagttatgtagtatttttagtttttttattaggattttattaatgttttttgtattggaaatttttgtgttagaggttgaatttatagtaatttttaaaattaattaagaagaatttagttagaggttatagtaatgttggaattataaaatgtataagatttatttttttttggttttttttttatttatgttgtttatgtttgtgtatttataagttttatgtatattaaatttttaaaattaattattattatgttatagagtttttattggatagtgttttttagtttttattatatatttttttttttttatgtagatttattatgttggtgttttgttatatagggggtttgagaagaatgttatttaattgttgttgttgtgagtgtgtaaagtgattaggagattaggagaatgttgaaatttttgttggaaaaatgtaaagaaaatttttattttgagttagttgtttatagagttagtgtgtgtgtgtgtgtgtgtgtttgtaatataaaatggatgtgaatatatatatataaatagatatggttttgtttttattttaatttgaattatttagataattgtttttatttattatttgattttaatgggtttatataaattaggatattttatttttttaggtatttaggttgttgttgatttttagtgtttttaatattttgtatatgttggtattatgaggagtagttatgtgtttttgggttttttaattattttggaggttgattgaggttttttatatatgtatatttgttgtgatgaaagttttattggtagagtggagttattagagtttttattaaaattttgtgggtttatgagagatgggtttagaaatttatatggttttgtggggttttttggttttttaaaataaggtattaatatttaagtttttaaaaatatttgtagttttggggtttgaattttgaaaaataaggagtgaggggttgtgtatattaattatagtggagattttttttattttttaatgtgatggagtttttttatgaaatgaagttttaaggggtatggtattgtggggattatagttattttgaggtttaaaagaagaaattggaatatgattagtaaatatatttagtagaaaagagttggatttttattgatttagttataggttattggttggtagtgtaatgggaggaaatatttattttatatatatattttatgattttgggggaattagaggaaatttaataagaaaatggttagaaatatttaaaatttttatttaaaagatttaagtaaattagagttttattagattaaaaattattataaatgtaagagtattgtttttagtgaaatgttgtggggtttgagaaggagattttttgttaaatttttgggataaaatgtgttatttaagtattagataatgagtagaatgtaaattaatttaattttttttattaataggttgttagtgtaatgtgtataatttagtgataagattgtaggatttaatatagttggatgtatgagttttagttaatgtagatttgttatatgaggatgtgttttattttgagtaggtgtttgtatgtgtggaatggggtaaagtggaataaaaggttaaaagtagaaatgttgatttaaagtttattatgaagaaattttttttttgtagttaaattatttttaaagtgggatgatattggtgaagaaagattgaaaaataatttttatgtgtgtttttggattgtaagtttaaaatggggaggagttgtagatagggtttgggggtggttagggtaaaggagagatatataagttgtaaatatatttgtagtttgttttatttattttgttttatattgaataagttttttaattttgtgaataaggataaggagggagtgttttaaagatattttatgttggtattgtaaattattgattgtaatgttaaataaatatatatttagagatgataatattaattttatagtaaaataattgtttatgtagaaatttagaggagattagtttgttttttttagttgatttatgttgggggataaaaggatttttaaaaattattttgaatatgtttggatttttttttttaatttttttggaaattaaatttgtttggaaatagtgttataaagagttgatgtttttaaaggtgattttttttgttttatataaataaggttttgtttttgttagttgagtgtagttttaggttttttgtttttagtttatatatattttttttgtttgtttggattttaatggtttaagatagttttgagtttattgggaaaagaaaatgattgttaaaaattatttttgaaattggttatttggtaatatttttaattgtatggaaatttattaaggtatattttatatataattagtttaaggttgttgattttataggttttatggatttaaatttgattgataataaagtaaataagagagttgaatttaaagtgtggtttttttgggttaggatgagtttaatatagtgtataaggaatttgaaagatttaggatatgtgttttaattaatgttaagtagaatggataagtttttagtattttgaaaatgttgggttagggttttttttttattgtgtgttttttgtttggggattaataagtattatagagaatgtgatttgaggtgattttttatttttgtataaatttagagtgaattattaaatagttgtttgtttaaagttaaggtaatttttttttgatgggtttatttgttttttgatttttaatttattagtttgtttttttagggttttgttttttttgtaattaaagtttttttagattagtgtagtatttatttgataggttgtttggaaaatttaagattggagaggtgatttgttgttgttttttaaattttttagttttaagtaatgtgttttttttttatatggggtgggggattggaaatggatgtagtgagatataaagagtgggtgttttgttgatttttgtatttttttttttttgattattttattttttttttttaagtttttgatttttagttttatttttttatttttgggtttgtattaaaagttggattgttttgggttgggtaggagttgaatttttgggagtttgtttgtgtagatttagtgtgtatggtgaggtagtagtttggttttgtattgttgataggtgtaggtaggatagtttttttattgtggtttggggtgttttgattggtgtggagttatgttagttgtatttggagaagggtttgggaggaggtggaggtggagagggttggggagggttgtggtggagtgatgttttggtattaggaagtttgtttttggttttaagatgttaggttaatagggaagtgtggagttgtagatttggtttgttgtttgtttgggtgtttggagttgagttgtggtaaggtttggtttttgtttgattgtttgaggggtgtgtgtgtgtgtgttgtggagggtgtgtttagagggttgtgttgtggttgtagtggttgttgttgttgtaggggatttaatattatttatttgtttttgttatttttgatatttttttgttagggttgttgtgtggggggggggtgggtagagtgtggttggtgttagttttttttattggaggggtttttgggggagggagggagagaagaagggggtttttgtttatttttgttttgttttggagtttggaagtttgttttttaaagatgttttgagtggtgtttttttgtttatattttatgtttttgtttgtttgttgattttttgtttttggatttttttgtttgagttttttggaggagatgggggtagtttggtttgagaatttggtgggggttgtgttttttggttttttttgtagtggggaaattttgtgtttagagtgtgatttggagtgggtagtggtggttatgggggtttggtggggtagtagttaaggattagtagagtgttgtgtttttttgtttatgaattgtatgaaaggtttgttttatttggagtattgagtagtggggattaagttgttggttgtttttttatttttttgttattatttttagttgttagttatggttttggttttggtttttggttagttttggttgttggatttttttaagtataggttggaggtgtatattatttttgatatttttagtttggaggttgtaggtaaggtgttgtgttgttttgtagatatttttgtttagttgttttgtgttatttgtttttttttgttttaaggaagttagtttttttggggggaggtgtggtgggagtggttgtttgtttggttttttgtagaatttttgggagttggaattttgattattttgtatttttttagtttttttttgattggtttggtttttggggtgttaagggtgtgagtaattttgttgttttttttatttgtattttggttttttttttgttttttgggttataaaaattttagtattttgatttgaggatttttagaggttgttgatttttgtttttgttttttttttggtttttagtttttgaggagttttatttgttaggaaattgtttgaaattatttagaaatgttttttgtgaagaggtattttttttttttttttgggaaagggttggtgaattttggtgtttaattgaatttttatatttttttttagtttttttaaattgtatggaaatttgagttttttgtgagggggaggggggtttgtaaattatgtgtgtgtgtgtgttttaggagatttggtgtgtttgtgtagaggtgtataaatatatttgaaagtataggttataaaagtgaatgtgttgttgtagtgagataaatatgtaaataaaatgtgtggtgttgggggaggggaggaaatggggtgtggatatttatatttgtgtttgtatattttataggtgtagtgttttttgtggtttggagttgttgtgtgtatttttttttggtgttaggtagtttagtttttttatggtttttgttgttggtttagttggtgtttgtgttgtaggtgggtatgttgatgggaaagtgtgtgtgttttgtttttagagaaagataaaagttagtaggggaagaatgaggatgtgggtgttgaggatttgtttaagaagaagtggtaaaggtggtagtggatttattttattagttagtagttttaggagttggaggttattttttagaggaattgttatttggatatgtttatatgtgaagaaattgttgtgtggattaattttatggaagtttgagtttgggtaggagttagtatggagtttgggagggatggggggaggatgttgtggaggtataggttaagtagattaggagagaatgtggaaggtagtgttgtttgggagggtgttggtggggtgtagttttgtaaaggtagaaggttttgtggtggtttggttgtgagattatagttttttttttgaggttgataggattgttgttttggtttaggtttttagagtggtattggtttattgttttgttattttgtgattttatgagttgggttgtatgggtaattttttgtataggatattgtgtttttggtttgtagttgttagagtagagttaataaaatttttattaggttaagagttgtgaataggttttaatttgtgagtttttaataaggaaaatttgttagagatatggaagagttggttttttttgggaaatttttgttttggttttggtttagtttttttttttttgggtttgtgttttttatattttttttatggttgttttggttatttaggttttttttatatattttattttttagttttgtgatttttgggagtaaagttttaatatataattattagtttttttagaaggagaaagaaaaaaagaagaaagatttttttgtttggtttatttatttttttttaggagttgaattttggaaattgaaatttatatttttttttttaaattataattatagttttgtaaaaagggtttattttaattttgtagtaaatttgtattttatggattggtaaaaatgagtttaaataaataatttaatagtaatgttttggtttatgttggttggtggaagattttaaatttgttaggattttggaagtagaaaatagaattaagtaaattaagtggtatttagaggttttgttgttaaaaaaaaaaaattaagtgttttgggtagaaaaaataaagtttttggttagagtagagtaaataaaaagaagaaaataatgataaaaagaataaagattaaaatgtttttttaaattagagggaatgaagatattttttgggtggtatttgtgtaaggtatgaggttatgttggtggataaaaggttgggaagaagttgaaaatggttttagtttaattgtttagagttagagttgggttttgggtggtgtggttttgagtaaggttagttttttattagtttttttgtatattaagggaatgggttttttatgtattttttttgtttgagtaaagtttagatggtttagggtagaaatggtaagtaattaaagatagagtttatgggttttttgggattttttgaaaatgtttttttattttgtttgttattttgtagttttattttagtgttttgtagttgtggtgttgggttttttttgtagttgtttttttttttagggtggttgtttgttgagttaagtgggagtgaggtgtgttttttatagtagttgggtgtaaagaggaagggggataaaaaggaaattaagaatgaaaggaaaaagagaaaaagtggattatatggttgggtttggtggagatgtgtaatgtgaaatattattggtgttagtttggatattttaggttaggtttttttttaatatataaaagttgttgtttggggtgatagggaggtttgatgtggattgggattggggttgtggttgggttattggatatgggtggaagttggttggtttgggtggttgtttgtaaagttaaatgatttggttgggtttggtgtgtggataggtttgtggtgggtttagggtaaagaagaggtagagtgaaagaagggggaatttttaaaattattttttttgggtttttggagtttaatatgttaagtttttggagttaatgagttgatgaagaggtggttttttgttttttatttggttgttttgttaggtgagaaagagtgttggtggtttagtttttgttaagggagtatgtattagggggtgggggatgatagtggaggttagggaaggaagggaggaattgtgtgggagaaagagtgattttttagtgtttttttagttttttttttttatttgtgggtttgtggttttggaatggaagtaagtttgtaaggtgttttgggaagggttggaaaagtttgttgttttgtgtttgttttatattaagtgtttttggatttggagaaatgtttggttgagtgattaaattgtttgtaggtttttatgtgtttggttgaggtttgtggtgtagttttgagttttagtttgtaggttagagtagattaggttttttgtgtttggtggagatttgggttagtaattgaaagttggttttggtattttggtgtgtagggtggtgtagtgaagtgaggttagggtgtgtgagtgtgttagtgtgtgtgttgggggaaggtgggggttggtttttgatggaagttttagtaatttgtattgtggtatttgtttgtttttttgttttaattgtttttaggtttggtttaagaattgttgggttaaatggagaaagagggagtgtaattagtaggttgagttatgtaagaatggttttgggttgtagtttaatgggtttatgtagttttatgatgatatgtatttaggttatttttataataattgggttgttaagggttttatatttgtttttttatttattaagagtttttttttttttaattttatgaatgttaattttttgttattatagagtatgttttttttatttaattttattttgtttatgagtatgttgtttagtatggtgtttttagtagtgataggtgttttgggttttagttttaatagtttgaataatttgaataatttgagtagtttgttgttgaattttgtggtgttgatgtttgtttgtttttatgtgttgttgatttttttgtatgtttatagggatatgtgtaatttgagtttggttagtttgagattgaaagtaaagtagtattttagttttggttatgttagtgtgtagaatttggtttttaatttgagtgtttgttagtatgtagtggattggtttgtgtgagttgtatttatagtgttgggattttaggattttgttggatggggtaattttgtttttgaaagattgggaattatgttagaaggttgtgggtattaaagaaagggagagaaagagaagttatatagagaaaaggaaattattgaattaaagagagagtttttttgattttaaagggatgtttttagtgtttgatattttttattataagtatttttaatagttgtaaggatatatatataaataaatgtttgattggatatgatattttaatattattataagtttgttattttttaagtttagtattgttaatatttaaatgattgaaaggatgtatatatattgaaatgttaaattaattttataaaagtagttgttagtaatattataatagtgtttttaaaggttaggttttaaaataaagtatgttatatagaagtgattaggattttttgtttgtgagtaagggagtgtatatattaaatgttatattgtatgtttttaatatattattattattataaaaaatgtgtgaatattagttttagaatagtttttttggtggatgtaatgatgtttttgaaattgttatgtataatttattttgtgtataatattttgtataatattattgttttattttttagtaaatatgaaataaatgtgttttattttatgggagtaaaatatattgtatataaattggtttggattttttttttttttttttgttattaatttggttaggatattttagttattgttttttaaataaattagttttttttgtttgtttagttaaatatataaggtagtagtttttatttaaatttggtagaaataaatgatagttatttattagaaattaaaaagaaaaaaaaaggtatttttgggggggaaaagggttataaaatttaattttgtttttttaatttttttttggtttaaatttagaggattttattatggttagtaaataatatgaaaaagaaaaaagaagaaagaaatttagtaagtttattagtttaaaatgatttttaagtttatttttttatggggaaatttatatttttagtaaattgttttggagaaatatttgtgtatgtatatatgtatagtttatatgtattttttttaggaggaatatatttataataaatttatagggaaatatttttagtttaaaatatttaggtttttatgtttatttttaggtttaagtagagagattttttatgttatattgtattattatttttaaattttttggagatattaaaagaaataaagatgatttttaataattatagttttttagttttttaaagaatttaggggttgagaggttagagtggagttttttgagttttgttgagtaatatgtagttgaggtaaaggttatgtttttggtgttttgttttaaataatattgatttattaattttaaatttgtttgtttttgaaattatataggattatagtttgtaaattgtaggataatgaagtaaattaagatgaattatagttttggttttttttgttattttttgatatttaaatagggaatgagtttggtgtgagtgtttaaatgaattttaagtatttgatttttttttatttgtgatttttagttttaaaaaaatgtgaaatttgattttataataaatagaaataaatattatttagttttagagaatttatttttatggtgttaggagggttgttgtggaggtggggggagggatgtgttgagattttttgttatgtttgttaattttttgtataattaaagtgggtgagaataaatattatgttggggaatttagagtaaaaagtaattgttgattttttggagttgataatattattgtttttttgttttagttgtttttatttgaatgaaattttatttagaagtttttggagtttgaatattgagtttttgttttgtaagaaattgttgttgttatttaaagagattgtagataatgtttttgatttaagattagtgtttaatgtatattttttttttttttaaagttttgtttttgatttgtggagggattatgtagtagtggggggtagataaaagttttttggatgagggttattttttattatgttgtttatttgagagtagtggagaggaaaatggtttttatgggtggattttggtttttggagttgtagggtttagtggttttggtttttttgttttttagtttggtagggggtggggagggttaaagggtggaggggaaggaaggagttagaagaggggatttggggatgggggttggaagtgttaatgagatttgtttggaggatttaggttttttgtaggttggtgagtgatttgggagttttgggtaaaagaggtgtattttggtttagtttggttgttgaattagaataatgtgaggatattaattaatttgtaggaaataaaaaatggaagttgaggtttaggaagagttgttatttttttgatttgagtggtgtaggttgggggtggagatttgggatttaagagaggttattttttttattttagttttttttttttggatttttaaaaggaataattttatttttttttttgttttttttataatttttatttttgttagtttgtaggttgtttgtttttttttgtattttttttttttatttagtgagagaaatttagtttttagagt